(19) United States
(12) Patent Application Publication
(10) Pub. No.: US 2021/0047324 A1
(43) Pub. Date: Feb. 18, 2021

(54) SPIRO-LACTAM NMDA RECEPTOR MODULATORS AND USES THEREOF

(71) Applicant: Aptinyx Inc., Evanston, IL (US)

(72) Inventor: M. Amin Khan, Evanston, IL (US)

(21) Appl. No.: 16/966,176

(22) PCT Filed: Jan. 31, 2019
(86) PCT No.: PCT/US2019/016098

§ 371 Date: Jul. 30, 2020

Related U.S. Application Data
(60) Provisional application No. 62/718,107, filed on Aug. 13, 2018.
(60) Provisional application No. 62/624,218, filed on Jan. 31, 2018.

(51) Int. Cl. C07D 471/10

(52) U.S. Cl. CPC C07D 471/10

(57) ABSTRACT

Disclosed are compounds having potency in the modulation of NMDA receptor activity. Such compounds can be useful in the treatment of conditions such as depression and related disorders as well as other disorders.

### CROSS REFERENCE TO RELATED APPLICATIONS

This application claims priority to and the benefit of U.S. Provisional Patent Application No. 62/718,107, filed on Aug. 13, 2018, and U.S. Provisional Patent Application No. 62/624,218, filed on Jan. 31, 2018; the contents of each of which are hereby incorporated by reference herein in their entirety.

### BACKGROUND

An N-methyl-d-aspartate (“NMDA”) receptor is a postsynaptic, ionotropic receptor that is responsive to, inter alia, the excitatory amino acids glutamate and glycine and the synthetic compound NMDA. The NMDA receptor controls the flow of both divalent and monovalent ions into the postsynaptic neural cell through a receptor associated channel (Foster et al., Nature 1987, 329:395-396; Mayer et al., Trends in Pharmacol. Sci. 1990, 11:254-260). The NMDA receptor has been implicated during development in specifying neuronal architecture and synaptic connectivity, and may be involved in experience-dependent synaptic modifications. In addition, NMDA receptors are also thought to be involved in long term potentiation and central nervous system disorders.

The NMDA receptor plays a major role in the synaptic plasticity that underlies many higher cognitive functions, such as memory acquisition, retention and learning, as well as in certain cognitive pathways and in the perception of pain (Collingridge et al., The NMDA Receptor, Oxford University Press, 1994). In addition, certain properties of NMDA receptors suggest that they may be involved in the information-processing in the brain that underlies consciousness itself.

The NMDA receptor has drawn particular interest since it appears to be involved in a broad spectrum of CNS disorders. For instance, during brain ischemia caused by stroke or traumatic injury, excessive amounts of the excitatory amino acid glutamate are released from damaged or oxygen deprived neurons. This excess glutamate binds to the NMDA receptors which opens their ligand-gated ion channels; in turn the calcium influx produces a high level of intracellular calcium which activates a biochemical cascade resulting in protein degradation and cell death. This phenomenon, known as excitotoxicity, is also thought to be responsible for the neurological damage associated with other disorders ranging from hypoglycemia and cardiac arrest to epilepsy. In addition, there are preliminary reports indicating similar involvement in the chronic neurodegeneration of Huntington's, Parkinson's and Parkinson's related conditions such as dyskinesia and L-dopa induced dyskinesia and Alzheimer's diseases. Activation of the NMDA receptor has been shown to be responsible for post-stroke convulsions, and, in certain models of epilepsy, activation of the NMDA receptor has been shown to be necessary for the generation of seizures. Neuropsychiatric involvement of the NMDA receptor has also been recognized since blockage of the NMDA receptor Ca⁺⁺ channel by the animal anesthetic PCP (phencyclidine) produces a psychotic state in humans similar to schizophrenia (reviewed in Johnson, K. and Jones, S., 1990). Further, NMDA receptors have also been implicated in certain types of spatial learning.

The NMDA receptor is believed to consist of several protein chains embedded in the postsynaptic membrane. The first two types of subunits discovered so far form a large extracellular region, which probably contains most of the allosteric binding sites, several transmembrane regions looped and folded so as to form a pore or channel, which is permeable to Ca⁺⁺, and a carboxyl terminal region. The opening and closing of the channel is regulated by the binding of various ligands to domains (allosteric sites) of the protein residing on the extracellular surface. The binding of the ligands is thought to affect a conformational change in the overall structure of the protein which is ultimately reflected in the channel opening, partially opening, partially closing, or closing.

A need continues to exist in the art for novel and more specific and/or potent compounds that are capable of modulating NMDA receptors, and provide pharmaceutical benefits. In addition, a need continues to exist in the medical arts for orally deliverable forms of such compounds.

### SUMMARY

The present disclosure includes compounds that can be NMDA modulators. More specifically, the present disclosure provides a compound having the formula:

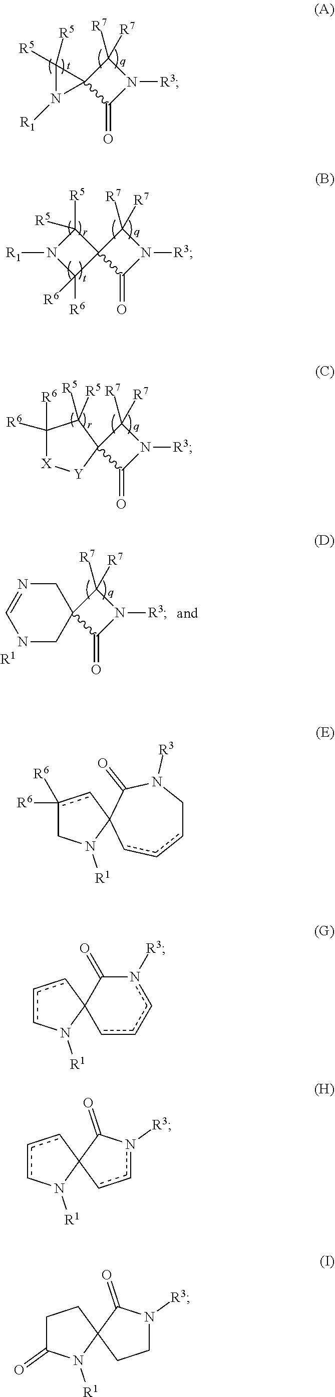

or a pharmaceutically acceptable salt and/or stereoisomer thereof, wherein:
R¹ is independently selected from the group consisting of H, —C₁-C₆alkyl, —C(O)—C₁-C₆alkyl, —C(O)—O—C₁-C₆alkyl, and —S(O)_{w}—C₁-C₆alkyl, wherein C₁-C₆alkyl is optionally substituted with one, two, or three substituents each independently selected from R^{S};
w is 0, 1 or 2;
R⁵ is independently selected for each occurrence from the group consisting of H, —C₁-C₆alkyl, and halogen, wherein C₁-C₆alkyl is optionally substituted with one, two, or three substituents each independently selected from R^{S};
R⁶ is independently selected for each occurrence from the group consisting of H, —C₁-C₆alkyl, and halogen, wherein C₁-C₆alkyl is optionally substituted with one, two, or three substituents each independently selected from R^{S}; or
R⁵ and R⁶, or two R⁵ moieties, when present on adjacent carbons, form a 3-membered carbocyclic ring taken together with the adjacent carbons to which they are attached, optionally substituted by one or two substituents independently selected from the group consisting of halogen, hydroxyl, —C₁-C₃alkyl, —C₁-C₃alkoxy, —C(O)NR^{a}R^{b}, and —NR^{a}R^{b};
R⁷ is independently selected for each occurrence from the group consisting of H, —C₁-C₆alkyl, phenyl, and halogen, wherein C₁-C₆alkyl is optionally substituted with one, two, or three substituents each independently selected from R^{S}, and phenyl is optionally substituted with one, two, or three substituents each independently selected from R^{T};
R³ is selected from the group consisting of H, —C₁-C₆alkyl, phenyl, —C(O)—R³¹, and —C(O)—O—R³², wherein C₁-C₆alkyl is optionally substituted with one, two, or three substituents each independently selected from R^{S}, and phenyl is optionally substituted with one, two, or three substituents each independently selected from R^{T};
R³¹ is selected from the group consisting of H, —C₁-C₆alkyl, —C₃-C₆cycloalkyl, and phenyl, wherein C₁-C₆alkyl is optionally substituted with one, two, or three substituents each independently selected from R^{S}, and each of C₃-C₆cycloalkyl and phenyl is optionally substituted with one, two, or three substituents each independently selected from R^{T};
R³² is selected from the group consisting of H, —C₁-C₆alkyl, —C₃-C₆cycloalkyl, and phenyl, wherein C₁-C₆alkyl is optionally substituted with one, two, or three substituents each independently selected from R^{S}, and phenyl is optionally substituted with one, two, or three substituents each independently selected from R^{T}; and
R^{a} and R^{b} are independently, for each occurrence, selected from the group consisting of H, —C(O)—O—CH₂-phenyl, and —C₁-C₃alkyl; or R^{a} and R^{b} taken together with the nitrogen to which they are attached form a 4-6 membered heterocyclic ring, wherein phenyl is optionally substituted with one, two, or three substituents each independently selected from R^{T};
R^{S} is independently, for each occurrence, selected from the group consisting of —C(O)NR^{a}R^{b}, —NR^{a}R^{b}, hydroxyl, —SH, phenyl, —O—CH₂-phenyl, and halogen, wherein each phenyl is optionally substituted with one, two, or three substituents each independently selected from the group consisting of —C₁-C₃alkoxy and halogen;
   R^{T} is independently, for each occurrence, selected from the group consisting of —C(O)NR^{a}R^{b}, —NR^{a}R^{b}, —C₁-C₃alkyl, —C₁-C₃alkoxy, hydroxyl, and halogen; and wherein
for Formula A:
   t is 1, and q is 1, 2, 3, 4, or 5; or;
   t is 2, 4 or 5, and q is 2, 3, 4, or 5; or,
   t is 3 and q is 3, 4, or 5;
for Formula B:
   t is 1, r is 1, and q is 1, 2, 3, 4, or 5; or
   t is 1, r is 2, and q is 1, 3, 4, or 5, or
   t is 1, r is 3, q is 3, 4, or 5, or
   t is 1, r is 4, q is 2, 3, 4, or 5; or
   t is 2, r is 3 or 4, q is 2, 3, 4, or 5;
for Formula C:
   r is 0, 1, or 2;
   q is 1, 2, 3, 4, or 5; and
   —X—Y— is selected from the group consisting of:

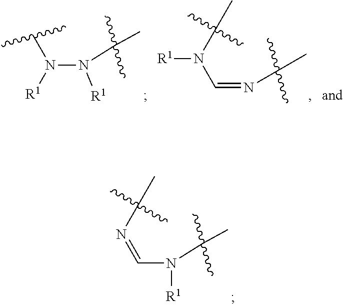

for Formula D:
   q is 1, 2, 3, 4, or 5; and
for Formula E:
   [symbol] is either a single or double bond;
   when a double bond is present in the 5-membered ring, only one R⁶ is present;
   the one double bond in the 7-membered ring is present between the α and β ring carbons or the β and γ ring carbons, with respect to the spiro junction;
for Formula G:
   [symbol] is either a single or double bond;
   there is one double bond in the 5-membered ring;
   there is one double bond in the 6-membered ring;
   if the double bond in the 6-membered ring is a C═N bond, then R³ is absent;
for Formula H:
   [symbol] is either a single or double bond;
   there is one double bond in the ring without a carbonyl group;
   there is one double bond in the ring with a carbonyl group; and
   if the double bond in the ring with a carbonyl group is a C═N bond, then R³ is absent.

Also provided herein is a compound having the formula:

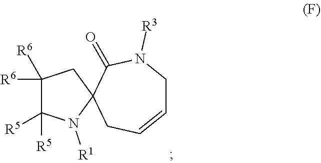

or a pharmaceutically acceptable salt and/or stereoisomer thereof, wherein
R¹ is independently selected from the group consisting of H, —C₁-C₄alkyl, —C(O)—C₁-C₄alkyl, —S(O)_{w}—C₁-C₄alkyl, and —C(O)—O—C₁-C₄alkyl, wherein C₁-C₄alkyl is optionally substituted with one, two, or three substituents each independently selected from R^{S};
w is 0, 1 or 2;
R⁵ is independently selected for each occurrence from the group consisting of H, —C₁-C₄alkyl, and halogen, wherein C₁-C₄alkyl is optionally substituted with one, two, or three substituents each independently selected from R^{S};
R⁶ is independently selected for each occurrence from the group consisting of H, —C₁-C₄alkyl, and halogen, wherein C₁-C₄alkyl is optionally substituted with one, two, or three substituents each independently selected from R^{S};
R³ is selected from the group consisting of H, —C₁-C₄alkyl, —C₁-C₄alkyl-phenyl, —C(O)—R³¹, and —C(O)—O—R³², wherein C₁-C₄alkyl is optionally substituted with one, two, or three substituents each independently selected from R^{S}, and phenyl is optionally substituted with one, two, or three substituents each independently selected from R^{T};
R³¹ is selected from the group consisting of H, —C₁-C₄alkyl, —C₃-C₆cycloalkyl, and phenyl, wherein C₁-C₄alkyl is optionally substituted with one, two, or three substituents each independently selected from R^{S}, and phenyl is optionally substituted with one, two, or three substituents each independently selected from R^{T};
R³² is selected from the group consisting of H, —C₁-C₄alkyl, —C₃-C₆cycloalkyl, and phenyl, wherein C₁-C₄alkyl is optionally substituted with one, two, or three substituents each independently selected from R^{S}, and phenyl is optionally substituted with one, two, or three substituents each independently selected from R^{T}; and
R^{a} and R^{b} are each independently for each occurrence selected from the group consisting of H, phenyl, and —C₁-C₄alkyl; or R^{a} and R^{b} taken together with the nitrogen to which they are attached form a 4-6 membered heterocyclic ring, wherein C₁-C₄alkyl is optionally substituted with one, two, or three substituents each independently selected from —C₁-C₃alkoxy, hydroxyl, and halogen;
R^{S} is independently, for each occurrence, selected from the group consisting of —C(O)NR^{a}R^{b}, —NR^{a}R^{b}, hydroxyl, —C(O)—O—R^{a}, phenyl, and halogen, wherein each phenyl is optionally substituted with one, two, or three substituents each independently selected from the group consisting of —C₁-C₃alkoxy and halogen; and
R^{T} is independently, for each occurrence selected from the group consisting of —C(O)NR^{a}R^{b}, —NR^{a}R^{b}, —C₁-C₃alkoxy, hydroxyl, and halogen.

Also provided herein are pharmaceutically acceptable compositions comprising a disclosed compound, and a pharmaceutically acceptable excipient. Such compositions can be suitable for administration to a patient orally, parenterally, topically, intravaginally, intrarectally, sublingually, ocularly, transdermally, or nasally.

In one aspect, a method of treating a condition selected from the group consisting of autism, anxiety, depression, bipolar disorder, attention deficit disorder, attention deficit hyperactivity disorder (ADHD), schizophrenia, a psychotic disorder, a psychotic symptom, social withdrawal, obsessive-compulsive disorder, phobia, post-traumatic stress disorder or syndrome, a behavior disorder, an impulse control disorder, a substance abuse disorder, a sleep disorder, a cognitive impairment disorder such as a memory disorder or a learning disorder, urinary incontinence, multiple system atrophy, progressive supra-nuclear palsy, Friedrich's ataxia, Down's syndrome, fragile X syndrome, tuberous sclerosis, olivio-ponto-cerebellar atrophy, Rett syndrome, cerebral palsy, drug-induced optic neuritis, ischemic retinopathy, diabetic retinopathy, glaucoma, dementia, AIDS dementia, Alzheimer's disease, Huntington's chorea, spasticity, myoclonus, muscle spasm, Tourette's syndrome, epilepsy, cerebral ischemia, stroke, a brain tumor, traumatic brain injury, cardiac arrest, myelopathy, spinal cord injury, peripheral neuropathy, fibromyalgia, acute neuropathic pain, and chronic neuropathic pain, in a patient in need thereof is provided. Such methods may comprise administering to a patient a therapeutically effective amount of a disclosed compound, or a pharmaceutically acceptable salt, a stereoisomer, and/or an N-oxide thereof, or a pharmaceutical composition including a disclosed compound, or a pharmaceutically acceptable salt, a stereoisomer, and/or an N-oxide thereof.

In various embodiments, a method of this disclosure includes treating depression. In some embodiments, a method of this disclosure includes treating schizophrenia. In certain embodiments, a method of this disclosure includes treating Alzheimer's disease. In various embodiments, a method of this disclosure includes treating attention deficit disorder. In some embodiments, a method of this disclosure includes treating anxiety. In certain embodiments, a method of this disclosure includes treating a migraine. In various embodiments, a method of this disclosure includes treating neuropathic pain. In some embodiments, a method of this disclosure includes treating traumatic brain injury. In certain embodiments, a method of this disclosure includes treating a neurodevelopment disorder related to a synaptic dysfunction. In various embodiments, a method of this disclosure includes treating a cognitive impairment disorder. Such methods may comprise administering to a patient a therapeutically effective amount of a disclosed compound, or a pharmaceutically acceptable salt, a stereoisomer, and/or an N-oxide thereof, or a pharmaceutical composition including a disclosed compound, or a pharmaceutically acceptable salt, a stereoisomer, and/or an N-oxide thereof.

### DETAILED DESCRIPTION

This disclosure is generally directed to compounds that are capable of modulating NMDA receptors, for example, NMDA receptor antagonists, agonists, or partial agonists, and compositions and/or methods of using the disclosed compounds. In some embodiments, compounds described herein bind to NMDA receptors expressing certain NR2 subtypes. In some embodiments, the compounds described herein bind to one NR2 subtype and not another.

It should be appreciated that the disclosed compounds may modulate other protein targets and/or specific NMDA receptor subtype.

The term “alkyl,” as used herein, refers to a saturated straight-chain or branched hydrocarbon, such as a straight-chain or branched group of 1-6, 1-4, or 1-3 carbon atoms, referred to herein as C₁-C₆ alkyl, C₁-C₄ alkyl, and C₁-C₃ alkyl, respectively. For example, “C₁-C₆alkyl” refers to a straight-chain or branched saturated hydrocarbon containing 1-6 carbon atoms. Examples of a C₁-C₆ alkyl group include, but are not limited to, methyl, ethyl, propyl, butyl, pentyl, hexyl, isopropyl, isobutyl, sec-butyl, tert-butyl, isopentyl, and neopentyl. In another example, “C₁-C₄ alkyl” refers to a straight-chain or branched saturated hydrocarbon containing 1-4 carbon atoms. Examples of a C₁-C₄ alkyl group include, but are not limited to, methyl, ethyl, propyl, butyl, isopropyl, isobutyl, sec-butyl and tert-butyl. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 3-methyl-2-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, and hexyl.

The term “alkoxy,” as used herein, refers to an alkyl group attached to an oxygen atom (alkyl-O—). Alkoxy groups can have 1-3, 1-4, 1-6 or 2-6 carbon atoms and are referred to herein as C₁-C₃ alkoxy, C₁-C₄ alkoxy, C₁-C₆ alkoxy, and C₂-C₆ alkoxy, respectively. Exemplary alkoxy groups include, but are not limited to, methoxy, ethoxy, propyloxy, isopropoxy, and tert-butoxy.

The term “carbonyl,” as used herein, refers to the radical —C(O)— or C═O.

The phrase, “carbocyclic ring,” as used herein, refers to a hydrocarbon ring system in which all the ring atoms are carbon. Exemplary carbocyclic rings including cycloalkyls and phenyl.

The term “cycloalkyl,” as used herein, refers to a monocyclic saturated or partially unsaturated hydrocarbon ring (carbocyclic) system, for example, where each ring is either completely saturated or contains one or more units of unsaturation, but where no ring is aromatic. A cycloalkyl can have 3-6 or 4-6 carbon atoms in its ring system, referred to herein as C₃-C₆ cycloalkyl or C₄-C₆ cycloalkyl, respectively. Exemplary cycloalkyl groups include, but are not limited to, cyclohexyl, cyclohexenyl, cyclopentyl, cyclopentenyl, cyclobutyl, and cyclopropyl.

The terms “halo” and “halogen,” as used herein, refer to fluoro (F), chloro (Cl), bromo (Br), and/or iodo (I).

The term “heteroatom,” as used herein, refers to an atom of any element other than carbon or hydrogen and includes, for example, nitrogen (N), oxygen (O), silicon (Si), sulfur (S), phosphorus (P), and selenium (Se).

The terms “hydroxy” and “hydroxyl,” as used herein, refer to the radical —OH.

The term “oxo,” as used herein, refers to the radical ═O (double bonded oxygen).

The term “amino acid,” as used herein, includes any one of the following alpha amino acids: isoleucine, alanine, leucine, asparagine, lysine, aspartate, methionine, cysteine, phenylalanine, glutamate, threonine, glutamine, tryptophan, glycine, valine, proline, arginine, serine, histidine, and tyrosine. An amino acid also can include other art-recognized amino acids such as beta amino acids.

The term “compound,” as used herein, refers to the compound itself and its pharmaceutically acceptable salts, hydrates, and N-oxides including its various stereoisomers and its isotopically-labelled forms, unless otherwise understood from the context of the description or expressly limited to one particular form of the compound, i.e., the compound itself, a specific stereoisomer and/or isotopically-labelled compound, or a pharmaceutically acceptable salt, a hydrate, or an N-oxide thereof. It should be understood that a compound can refer to a pharmaceutically acceptable salt, or a hydrate, or an N-oxide of a stereoisomer of the compound and/or an isotopically-labelled compound.

The term “moiety,” as used herein, refers to a portion of a compound or molecule.

The compounds of the disclosure can contain one or more chiral centers and/or double bonds and therefore, can exist as stereoisomers, such as geometric isomers, and enantiomers or diastereomers. The term “stereoisomers,” when used herein, consists of all geometric isomers, enantiomers and/or diastereomers of the compound. For example, when a compound is shown with specific chiral center(s), the compound depicted without such chirality at that and other chiral centers of the compound are within the scope of the present disclosure, i.e., the compound depicted in two-dimensions with “flat” or “straight” bonds rather than in three dimensions, for example, with solid or dashed wedge bonds. Stereospecific compounds may be designated by the symbols “R” or “S,” depending on the configuration of substituents around the stereogenic carbon atom. The present disclosure encompasses all the various stereoisomers of these compounds and mixtures thereof. Mixtures of enantiomers or diastereomers can be designated “(±)” in nomenclature, but a skilled artisan will recognize that a structure can denote a chiral center implicitly. It is understood that graphical depictions of chemical structures, e.g., generic chemical structures, encompass all stereoisomeric forms of the specified compounds, unless indicated otherwise.

Individual enantiomers and diastereomers of compounds of the present disclosure can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, (3) direct separation of the mixture of optical enantiomers on chiral liquid chromatographic columns, or (4) kinetic resolution using stereoselective chemical or enzymatic reagents. Racemic mixtures also can be resolved into their component enantiomers by well-known methods, such as chiral-phase gas chromatography or crystallizing the compound in a chiral solvent. Stereoselective syntheses, a chemical or enzymatic reaction in which a single reactant forms an unequal mixture of stereoisomers during the creation of a new stereocenter or during the transformation of a pre-existing one, are well known in the art. Stereoselective syntheses encompass both enantio- and diastereoselective transformations. See, for example, Carreira and Kvaerno, Classics in Stereoselective Synthesis, Wiley-VCH: Weinheim, 2009.

Geometric isomers, resulting from the arrangement of substituents around a carbon-carbon double bond or arrangement of substituents around a cycloalkyl or heterocycloalkyl, can also exist in the compounds of the present disclosure. The symbol [symbol] denotes a bond that may be a single, double or triple bond as described herein. Substituents around a carbon-carbon double bond are designated as being in the “Z” or “E” configuration, where the terms “Z” and “E” are used in accordance with IUPAC standards. Unless otherwise specified, structures depicting double bonds encompass both the “E” and “Z” isomers.

Substituents around a carbon-carbon double bond alternatively can be referred to as “cis” or “trans,” where “cis” represents substituents on the same side of the double bond and “trans” represents substituents on opposite sides of the double bond. The arrangement of substituents around a carbocyclic ring can also be designated as “cis” or “trans.” The term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

The disclosure also embraces isotopically-labeled compounds which are identical to those compounds recited herein, except that one or more atoms are replaced by an atom having an atomic mass or mass number different from the atomic mass or mass number usually found in nature. Examples of isotopes that can be incorporated into compounds described herein include isotopes of hydrogen, carbon, nitrogen, oxygen, phosphorus, fluorine and chlorine, such as ²H (“D”), ³H, ¹³C, ¹⁴C, ¹⁵N, ¹⁸O, ¹⁷O, ³¹P, ³²P, ³⁵S, ¹⁸F, and ³⁶Cl, respectively. For example, a compound described herein can have one or more H atoms replaced with deuterium.

Certain isotopically-labeled compounds (e.g., those labeled with ³H and ¹⁴C) can be useful in compound and/or substrate tissue distribution assays. Tritiated (i.e., ³H) and carbon-14 (i.e., ¹⁴C) isotopes can be particularly preferred for their ease of preparation and detectability. Further, substitution with heavier isotopes such as deuterium (i.e., ²H) can afford certain therapeutic advantages resulting from greater metabolic stability (e.g., increased in vivo half-life or reduced dosage requirements) and hence can be preferred in some circumstances. Isotopically-labeled compounds can generally be prepared by following procedures analogous to those disclosed herein, for example, in the Examples section, by substituting an isotopically-labeled reagent for a non-isotopically-labeled reagent.

The phrases “pharmaceutically acceptable” and “pharmacologically acceptable,” as used herein, refer to compounds, molecular entities, compositions, materials, and/or dosage forms that do not produce an adverse, allergic or other untoward reaction when administered to an animal, or a human, as appropriate. For human administration, preparations should meet sterility, pyrogenicity, general safety and purity standards as required by FDA Office of Biologics standards.

The phrases “pharmaceutically acceptable carrier” and “pharmaceutically acceptable excipient,” as used herein, refer to any and all solvents, dispersion media, coatings, isotonic and absorption delaying agents, and the like, that are compatible with pharmaceutical administration. Pharmaceutical acceptable carriers can include phosphate buffered saline solution, water, emulsions (e.g., such as an oil/water or water/oil emulsions), and various types of wetting agents. The compositions also can include stabilizers and preservatives.

The phrase “pharmaceutical composition,” as used herein, refers to a composition comprising at least one compound as disclosed herein formulated together with one or more pharmaceutically acceptable carriers. The pharmaceutical compositions can also contain other active compounds providing supplemental, additional, or enhanced therapeutic functions.

The terms “individual,” “patient,” and “subject,” as used herein, are used interchangeably and include any animal, including mammals, preferably mice, rats, other rodents, rabbits, dogs, cats, swine, cattle, sheep, horses, or primates, and more preferably, humans. The compounds described in the disclosure can be administered to a mammal, such as a human, but can also be administered to other mammals such as an animal in need of veterinary treatment, for example, domestic animals (e.g., dogs, cats, and the like), farm animals (e.g., cows, sheep, pigs, horses, and the like) and laboratory animals (e.g., rats, mice, guinea pigs, and the like). The mammal treated in the methods described in the disclosure is preferably a mammal in which treatment, for example, of pain or depression, is desired.

The term “treating,” as used herein, includes any effect, for example, lessening, reducing, modulating, ameliorating, or eliminating, that results in the improvement of the condition, disease, disorder, and the like, including one or more symptoms thereof. Treating can be curing, improving, or at least partially ameliorating the disorder.

The term “disorder” refers to and is used interchangeably with, the terms “disease,” “condition,” or “illness,” unless otherwise indicated.

The term “modulation,” as used herein, refers to and includes antagonism (e.g., inhibition), agonism, partial antagonism, and/or partial agonism.

The phrase “therapeutically effective amount,” as used herein, refers to the amount of a compound (e.g., a disclosed compound) that will elicit the biological or medical response of a tissue, system, animal or human that is being sought by the researcher, veterinarian, medical doctor or other clinician. The compounds described in the disclosure can be administered in therapeutically effective amounts to treat a disease. A therapeutically effective amount of a compound can be the quantity required to achieve a desired therapeutic and/or prophylactic effect, such as an amount which results in lessening of a symptom of a disease such as depression.

As used herein, the term “pharmaceutically acceptable salt” refers to any salt of an acidic or a basic group that may be present in a compound of the present disclosure, which salt is compatible with pharmaceutical administration. As is known to those of skill in the art, “salts” of the compounds of the present disclosure may be derived from inorganic or organic acids and bases.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present disclosure compounded with a suitable cation such as Na⁺, NH₄⁺, and NW₄⁺ (where W can be a C₁₋₄ alkyl group), and the like. For therapeutic use, salts of the compounds of the present disclosure can be pharmaceutically acceptable. However, salts of acids and bases that are non-pharmaceutically acceptable may also find use, for example, in the preparation or purification of a pharmaceutically acceptable compound.

Compounds included in the present compositions that are basic in nature are capable of forming a wide variety of salts with various inorganic and organic acids. The acids that can be used to prepare pharmaceutically acceptable acid addition salts of such basic compounds are those that form non-toxic acid addition salts, i.e., salts containing pharmacologically acceptable anions, including but not limited to, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts.

Compounds included in the present compositions that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

Compounds included in the present compositions that include a basic or acidic moiety can also form pharmaceutically acceptable salts with various amino acids. The compounds of the disclosure can contain both acidic and basic groups; for example, one amino and one carboxylic acid group. In such a case, the compound can exist as an acid addition salt, a zwitterion, or a base salt.

The compounds disclosed herein can exist in a solvated form as well as an unsolvated form with pharmaceutically acceptable solvents such as water, ethanol, and the like, and it is intended that the disclosure embrace both solvated and unsolvated forms Unless defined otherwise, all technical and scientific terms used herein have the same meaning as commonly understood by one of ordinary skill in the art to which this disclosure pertains.

Throughout the description, where compositions and kits are described as having, including, or comprising specific components, or where processes and methods are described as having, including, or comprising specific steps, it is contemplated that, additionally, there are compositions and kits of the present disclosure that consist essentially of, or consist of, the recited components, and that there are processes and methods according to the present disclosure that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present disclosure, whether explicit or implicit herein. For example, where reference is made to a particular compound, that compound can be used in various embodiments of compositions of the present disclosure and/or in methods of the present disclosure, unless otherwise understood from the context. In other words, within this application, embodiments have been described and depicted in a way that enables a clear and concise application to be written and drawn, but it is intended and will be appreciated that embodiments can be variously combined or separated without parting from the present teachings and disclosure(s). For example, it will be appreciated that all features described and depicted herein can be applicable to all aspects of the disclosure(s) described and depicted herein.

The articles “a” and “an” are used in this disclosure to refer to one or more than one (i.e., to at least one) of the grammatical object of the article, unless the context is inappropriate.

By way of example, “an element” means one element or more than one element.

The term “and/or” is used in this disclosure to mean either “and” or “or” unless indicated otherwise.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present disclosure also include the specific quantitative value itself, unless specifically stated otherwise. As used herein, the term “about” refers to a ±10% variation from the nominal value unless otherwise indicated or inferred from the context.

Where a percentage is provided with respect to an amount of a component or material in a composition, the percentage should be understood to be a percentage based on weight, unless otherwise stated or understood from the context.

Where a molecular weight is provided and not an absolute value, for example, of a polymer, then the molecular weight should be understood to be an average molecule weight, unless otherwise stated or understood from the context.

It should be understood that the order of steps or order for performing certain actions is immaterial so long as the present disclosure remain operable. Moreover, two or more steps or actions can be conducted simultaneously.

At various places in the present specification, substituents are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl. By way of other examples, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20. Additional examples include that the phrase “optionally substituted with 1-5 substituents” is specifically intended to individually disclose a chemical group that can include 0, 1, 2, 3, 4, 5, 0-5, 0-4, 0-3, 0-2, 0-1, 1-5, 1-4, 1-3, 1-2, 2-5, 2-4, 2-3, 3-5, 3-4, and 4-5 substituents.

The use of any and all examples, or exemplary language herein, for example, “such as” or “including,” is intended merely to illustrate better the present disclosure and does not pose a limitation on the scope of the disclosure unless claimed. No language in the specification should be construed as indicating any non-claimed element as essential to the practice of the present disclosure.

Further, if a variable is not accompanied by a definition, then the variable is defined as found elsewhere in the disclosure unless understood to be different from the context. In addition, the definition of each variable and/or substituent, for example, C₁-C₆ alkyl, R², R^{b}, w and the like, when it occurs more than once in any structure or compound, can be independent of its definition elsewhere in the same structure or compound.

Definitions of the variables and/or substituents in formulae and/or compounds herein encompass multiple chemical groups. The present disclosure includes embodiments where, for example, i) the definition of a variable and/or substituent is a single chemical group selected from those chemical groups set forth herein, ii) the definition is a collection of two or more of the chemical groups selected from those set forth herein, and iii) the compound is defined by a combination of variables and/or substituents in which the variables and/or substituents are defined by (i) or (ii).

Various aspects of the disclosure are set forth herein under headings and/or in sections for clarity; however, it is understood that all aspects, embodiments, or features of the disclosure described in one particular section are not to be limited to that particular section but rather can apply to any aspect, embodiment, or feature of the present disclosure.

### Compounds

Disclosed compounds include a compound having a formula selected from the group consisting of:

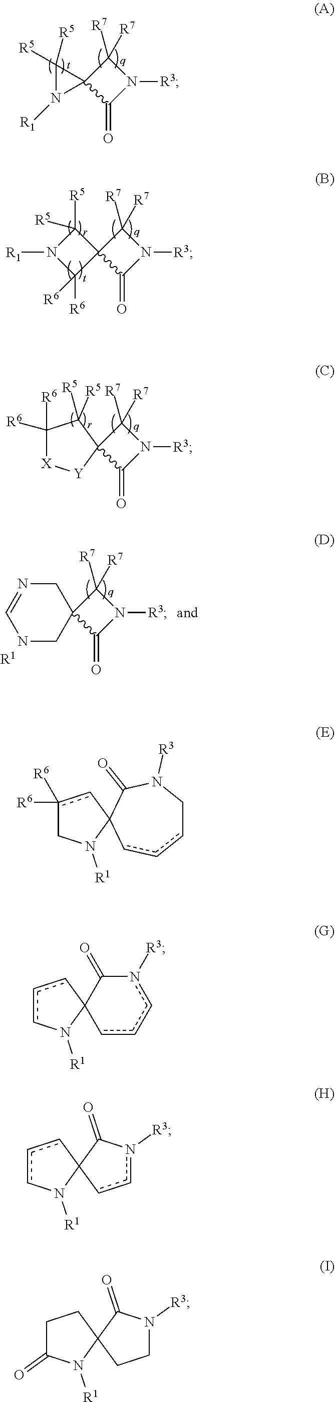

or a pharmaceutically acceptable salt and/or stereoisomer thereof, wherein
R¹ is independently selected from the group consisting of H, —C₁-C₆alkyl, —C(O)—C₁-C₆alkyl, —C(O)—O—C₁-C₆alkyl, and —S(O)_{w}—C₁-C₆alkyl, wherein C₁-C₆alkyl is optionally substituted with one, two, or three substituents each independently selected from R^{S};
w is 0, 1 or 2;
R⁵ is independently selected for each occurrence from the group consisting of H, —C₁-C₆alkyl, and halogen, wherein C₁-C₆alkyl is optionally substituted with one, two, or three substituents each independently selected from R^{S};
R⁶ is independently selected for each occurrence from the group consisting of H, —C₁-C₆alkyl, and halogen, wherein C₁-C₆alkyl is optionally substituted with one, two, or three substituents each independently selected from R^{S}; or
R⁵ and R⁶, or two R⁵ moieties, when present on adjacent carbons, form a 3-membered carbocyclic ring taken together with the adjacent carbons to which they are attached, optionally substituted by one or two substituents independently selected from the group consisting of halogen, hydroxyl, —C₁-C₃alkyl, —C₁-C₃alkoxy, —C(O)NR^{a}R^{b}, and —NR^{a}R^{b};
R⁷ is independently selected for each occurrence from the group consisting of H, —C₁-C₆alkyl, phenyl, and halogen, wherein C₁-C₆alkyl is optionally substituted with one, two, or three substituents each independently selected from R^{S}, and phenyl is optionally substituted with one, two, or three substituents each independently selected from R^{T};
R³ is selected from the group consisting of H, —C₁-C₆alkyl, phenyl, —C(O)—R³¹, and —C(O)—O—R³², wherein C₁-C₆alkyl is optionally substituted with one, two, or three substituents each independently selected from R^{S}, and phenyl is optionally substituted with one, two, or three substituents each independently selected from R^{T};
R³¹ is selected from the group consisting of H, —C₁-C₆alkyl, —C₃-C₆cycloalkyl, and phenyl, wherein C₁-C₆alkyl is optionally substituted with one, two, or three substituents each independently selected from R^{S}, and each of C₃-C₆cycloalkyl and phenyl is optionally substituted with one, two, or three substituents each independently selected from R^{T};
R³² is selected from the group consisting of H, —C₁-C₆alkyl, —C₃-C₆cycloalkyl, and phenyl, wherein C₁-C₆alkyl is optionally substituted with one, two, or three substituents each independently selected from R^{S}, and phenyl is optionally substituted with one, two, or three substituents each independently selected from R^{T}; and
R^{a} and R^{b} are independently, for each occurrence, selected from the group consisting of H, —C(O)—O—CH₂-phenyl, and —C₁-C₃alkyl; or R^{a} and R^{b} taken together with the nitrogen to which they are attached form a 4-6 membered heterocyclic ring, wherein phenyl is optionally substituted with one, two, or three substituents each independently selected from R^{T};
R^{S} is independently, for each occurrence, selected from the group consisting of —C(O)NR^{a}R^{b}, —NR^{a}R^{b}, hydroxyl, —SH, phenyl, —O—CH₂-phenyl, and halogen, wherein each phenyl is optionally substituted with one, two, or three substituents each independently selected from the group consisting of —C₁-C₃alkoxy and halogen;
R^{T} is independently, for each occurrence, selected from the group consisting of —C(O)NR^{a}R^{b}, —NR^{a}R^{b}, —C₁-C₃alkyl, —C₁-C₃alkoxy, hydroxyl, and halogen; and
   wherein
for Formula A:
   t is 1, and q is 1, 2, 3, 4, or 5; or;
   t is 2, 4 or 5, and q is 2, 3, 4, or 5; or,
   t is 3 and q is 3, 4, or 5;
for Formula B:
   t is 1, r is 1, and q is 1, 2, 3, 4, or 5; or
   t is 1, r is 2, and q is 1, 3, 4, or 5, or
   t is 1, r is 3, q is 3, 4, or 5, or
   t is 1, r is 4, q is 2, 3, 4, or 5; or
   t is 2, r is 3 or 4, q is 2, 3, 4, or 5;
for Formula C:
   r is 0, 1, or 2;
   q is 1, 2, 3, 4, or 5; and
   —X—Y— is selected from the group consisting of:

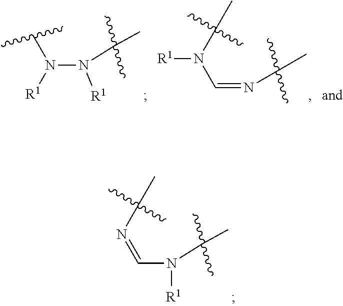

for Formula D:
   q is 1, 2, 3, 4, or 5; and
for Formula E:
   [symbol] is either a single or double bond;
   when a double bond is present in the 5-membered ring, only one R⁶ is present; and
   the one double bond in the 7-membered ring is present between the α and β ring carbons or the β and γ ring carbons, with respect to the spiro junction;
for Formula G:
   [symbol] is either a single or double bond;
   there is one double bond in the 5-membered ring;
   there is one double bond in the 6-membered ring;
   if the double bond in the 6-membered ring is a C═N bond, then R³ is absent;
for Formula H:
   [symbol] is either a single or double bond;
   there is one double bond in the ring without a carbonyl group;
   there is one double bond in the ring with a carbonyl group; and
   if the double bond in the ring with a carbonyl group is a C═N bond, then R³ is absent.

In particular embodiments, the compound can have the formula:

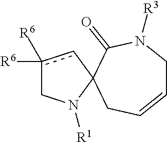

wherein the variables are as defined herein.

In certain embodiments, R¹ may be H. In other embodiments, R¹ may be —C(O)—O—C₁-C₆alkyl. For example, R¹ may be —C(O)—O-tert-butyl.

In certain embodiments, R¹ may be —C(O)—C₁-C₆alkyl. For example R¹ may be selected from the group consisting of:

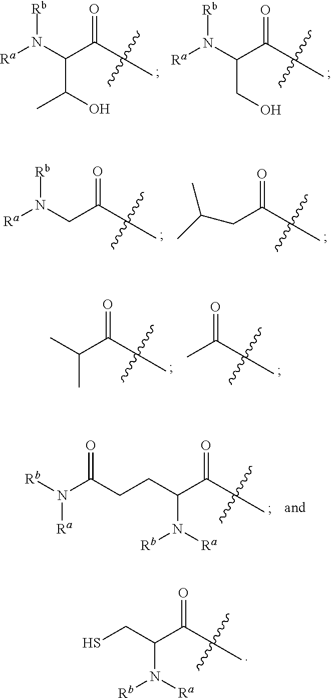

In various embodiments, R^{a} and R^{b} may be H. In certain embodiments, one of R^{a} and R^{b} is H and the other of R^{a} and R^{b} is methyl. In certain embodiments, each of R^{a} and R^{b} is methyl.

In certain embodiments, R¹ may be —C₁-C₆ alkyl optionally substituted by one, two or three substituents independently selected from the group consisting of —C(O)NR^{a}R^{b}, hydroxyl, —SH, halogen, and phenyl, wherein phenyl may be optionally substituted by one, two, or three substituents each independently selected from the group consisting of —C₁-C₃alkoxy and halogen. For example, R¹ may selected from the group consisting of:

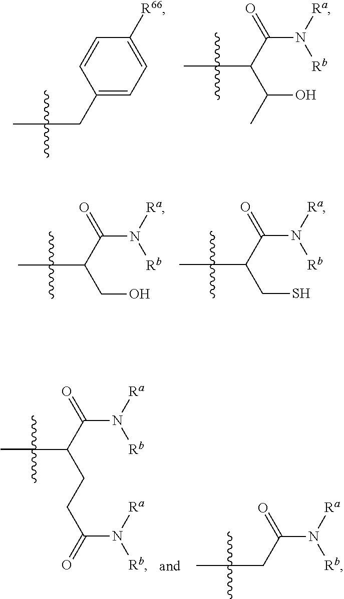

wherein R⁶⁶ is —C₁-C₃alkoxy or halogen and R^{a} and R^{b} are each independently selected for each occurrence from the group consisting of H and —C₁-C₆alkyl. For example, R⁶⁶ may be methoxy or fluoro (F).

In some embodiments, R¹ may be methyl.

In various embodiments, R¹ may be —S(O)_{w}—C₁-C₆alkyl, for example, —S(O)₂CH₃.

In some embodiments, R^{a} and R^{b} may be H.

In certain embodiments, R⁵ may be H. In some embodiments, one or two of R⁵ may be fluoro (F).

In various embodiments, R⁶ may be H. In some embodiments, one or two of R⁶ may be fluoro(F). In some embodiments, R⁵ and R⁶ may be H.

In certain embodiments, R³ may be H.

In various embodiments, R³ may be —C₁-C₆alkyl optionally substituted by one, two or three substituents independently selected from the group consisting of —C(O)NR^{a}R^{b}, hydroxyl, —SH, halogen, and phenyl, wherein phenyl may be optionally substituted by one, two, or three substituents each independently selected from the group consisting of —C₁-C₃alkoxy and halogen. For example, R³ may selected from the group consisting of:

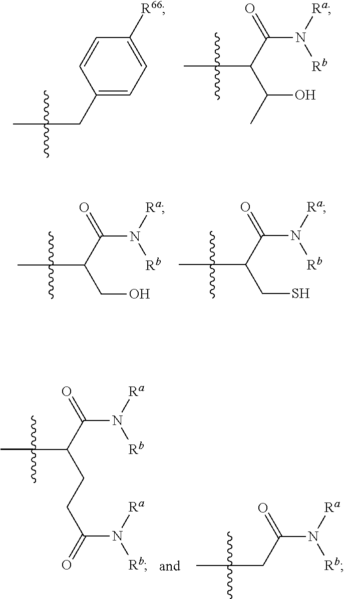

wherein R⁶⁶ is —C₁-C₃alkoxy or halogen; and R^{a} and R^{b} are each independently selected for each occurrence from the group consisting of H and —C₁-C₆alkyl. For example, R⁶⁶ may be methoxy or fluoro (F).

In certain embodiments, R^{a} and R^{b} may be H. In certain embodiments, one of R^{a} and R^{b} is H and the other of R^{a} and R^{b} is methyl. In certain embodiments, each of R^{a} and R^{b} is methyl.

In some embodiments, R³ may be methyl.

Disclosed compounds also include a compound having a formula:

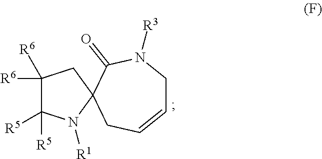

or a pharmaceutically acceptable salt and/or stereoisomer thereof, wherein
R¹ is independently selected from the group consisting of H, —C₁-C₄alkyl, —C(O)—C₁-C₄alkyl, —S(O)_{w}—C₁-C₄alkyl, and —C(O)—O—C₁-C₄alkyl, wherein C₁-C₄alkyl is optionally substituted with one, two, or three substituents each independently selected from R^{S};
w is 0, 1 or 2;
R⁵ is independently selected for each occurrence from the group consisting of H, —C₁-C₄alkyl, and halogen, wherein C₁-C₄alkyl is optionally substituted with one, two, or three substituents each independently selected from R^{S};
R⁶ is independently selected for each occurrence from the group consisting of H, —C₁-C₄alkyl, and halogen, wherein C₁-C₄alkyl is optionally substituted with one, two, or three substituents each independently selected from R^{S};
R³ is selected from the group consisting of H, —C₁-C₄alkyl, —C₁-C₄alkyl-phenyl, —C(O)—R³¹, and —C(O)—O—R³², wherein C₁-C₄alkyl is optionally substituted with one, two, or three substituents each independently selected from R^{S}, and phenyl is optionally substituted with one, two, or three substituents each independently selected from R^{T};
R³¹ is selected from the group consisting of H, —C₁-C₄alkyl, —C₃-C₆cycloalkyl, and phenyl, wherein C₁-C₄alkyl is optionally substituted with one, two, or three substituents each independently selected from R^{S}, and phenyl is optionally substituted with one, two, or three substituents each independently selected from R^{T};
R³² is selected from the group consisting of H, —C₁-C₄alkyl, —C₃-C₆cycloalkyl, and phenyl, wherein C₁-C₄alkyl is optionally substituted with one, two, or three substituents each independently selected from R^{S}, and phenyl is optionally substituted with one, two, or three substituents each independently selected from R^{T}; and
R^{a} and R^{b} are each independently for each occurrence selected from the group consisting of H, phenyl, and —C₁-C₄alkyl; or R^{a} and R^{b} taken together with the nitrogen to which they are attached form a 4-6 membered heterocyclic ring, wherein C₁-C₄alkyl is optionally substituted with one, two, or three substituents each independently selected from —C₁-C₃alkoxy, hydroxyl, and halogen;
R^{S} is independently, for each occurrence, selected from the group consisting of —C(O)NR^{a}R^{b}, —NR^{a}R^{b}, hydroxyl, —C(O)—O—R^{a}, phenyl, and halogen, wherein each phenyl is optionally substituted with one, two, or three substituents each independently selected from the group consisting of —C₁-C₃alkoxy and halogen; and
   R^{T} is independently, for each occurrence selected from the group consisting of —C(O)NR^{a}R^{b}, —NR^{a}R^{b}, —C₁-C₃alkoxy, hydroxyl, and halogen.

In certain embodiments, R¹ may be H.

In some embodiments, R¹ may be C₁-C₄alkyl, for example, methyl. In some embodiments, R¹ may be C₁-C₄alkyl, optionally substituted with one, two, or three substituents each independently selected from R^{S}. For example, R¹ may be —CH₂C(O)NH₂.

In particular embodiments, R¹ may be —CH₂-phenyl, optionally substituted by halogen or C₁-C₃alkoxy.

In various embodiments, R¹ may be —C(O)—C₁-C₄alkyl. For example R¹ may be selected from the group consisting of:

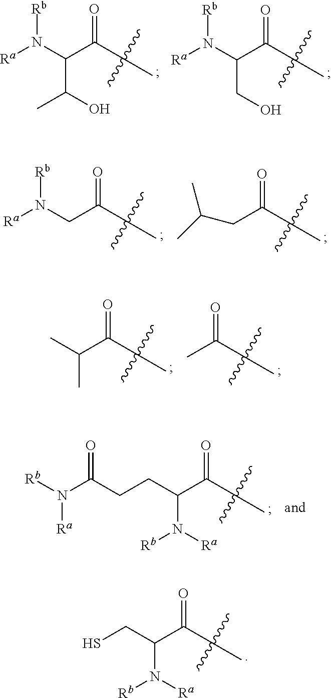

In various embodiments, R^{a} and R^{b} may be H. In certain embodiments, one of R^{a} and R^{b} is H and the other of R^{a} and R^{b} is methyl. In certain embodiments, each of R^{a} and R^{b} is methyl.

In certain embodiments, R¹ may be

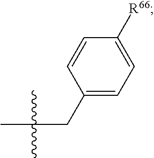

where R⁶⁶ may be selected from the group consisting of H, halogen and C₁-C₃alkoxy. In certain embodiments, R⁶⁶ may be F. In various embodiments, R⁶⁶ may be methoxy.

In various embodiments, R⁵ may be H. In certain embodiments, R⁶ may be H. In other embodiments, one or two of R⁶ may be fluoro. In certain embodiments, R⁵ and R⁶ may be H.

In certain embodiments, R³ may be H. In other embodiments, R³ may be methyl.

In certain embodiments, R³ may be

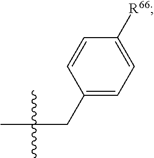

where R⁶⁶ may be selected from the group consisting of H, halogen and —C₁-C₃alkoxy. In some embodiments, R⁶⁶ may be fluoro (F). In some embodiments, R⁶⁶ may be methoxy.

In certain embodiments, R¹ and/or R³ independently can be an amino acid or a derivative of an amino acid, for example, an alpha “amino amide” represented by H₂N—CH(amino acid side chain)-C(O)NH₂. In certain embodiments, the nitrogen atom of the amino group of the amino acid or the amino acid derivative is a ring nitrogen in a chemical formula described herein. In such embodiments, the carboxylic acid of the amino acid or the amide group of an amino amide (amino acid derivative) is not within the ring structure, i.e., not a ring atom. In certain embodiments, the carboxylic acid group of the amino acid or the amino acid derivative forms an amide bond with a ring nitrogen in a chemical formula disclosed herein, thereby providing an amino amide, where the amino group of the amino amide is not within the ring structure, i.e., not a ring atom. In certain embodiments, R¹ and/or R³ independently can be an alpha amino acid, an alpha amino acid derivative, and/or another amino acid or amino acid derivative such as a beta amino acid or a beta amino acid derivative, for example, a beta amino amide.

In certain embodiments, a disclosed compound is selected from the compounds delineated in the Examples or tables herein, and includes a pharmaceutically acceptable salt and/or a stereoisomer thereof.

In particular embodiments, a disclosed compound is selected from the group consisting of:

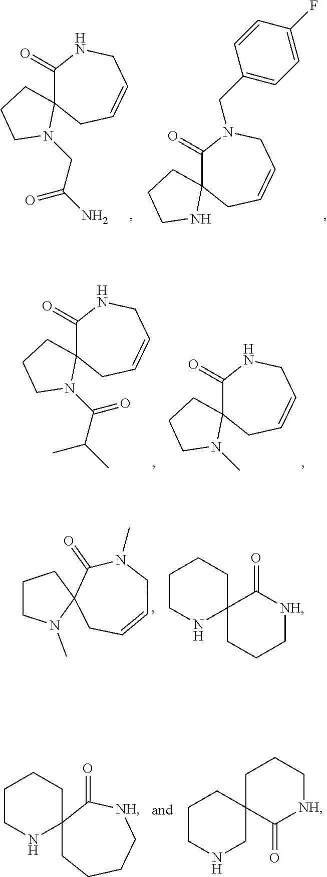

or a pharmaceutically acceptable salt and/or stereoisomer thereof

The compounds of the present disclosure and formulations thereof may have a plurality of chiral centers. Each chiral center may be independently R, S, or any mixture of R and S. For example, in some embodiments, a chiral center may have an R:S ratio of between about 100:0 and about 50:50 (“racemate”), between about 100:0 and about 75:25, between about 100:0 and about 85:15, between about 100:0 and about 90:10, between about 100:0 and about 95:5, between about 100:0 and about 98:2, between about 100:0 and about 99:1, between about 0:100 and 50:50, between about 0:100 and about 25:75, between about 0:100 and about 15:85, between about 0:100 and about 10:90, between about 0:100 and about 5:95, between about 0:100 and about 2:98, between about 0:100 and about 1:99, between about 75:25 and 25:75, and about 50:50. Formulations of the disclosed compounds comprising a greater ratio of one or more isomers (i.e., R and/or S) may possess enhanced therapeutic characteristic relative to racemic formulations of a disclosed compounds or mixture of compounds. In some instances, chemical formulas contain the descriptor “—(R)—” or “—(S)—” that is further attached to solid wedge or dashed wedge. This descriptor is intended to show a methine carbon (CH) that is attached to three other substituents and has either the indicated R or S configuration.

Disclosed compounds may provide for efficient cation channel opening at the NMDA receptor, e.g., may bind or associate with the glutamate site or glycine site or other modulatory site of the NMDA receptor to assist in opening the cation channel. The disclosed compounds may be used to regulate (turn on or turn off) the NMDA receptor through action as an agonist or antagonist.

The compounds described herein, in some embodiments, may bind to a specific N-methyl-D-aspartate (NMDA) receptor subtypes. For example, a disclosed compound may bind to one NMDA subtype and not another. In another embodiment, a disclosed compound may bind to one, or more than one NMDA subtype, and/or may have substantially less (or substantial no) binding activity to certain other NMDA subtypes. For example, in some embodiments, a disclosed compound (e.g., compound A) binds to NR2A with substantially no binding to NR2D. In some embodiments, a disclosed compound (e.g., compound B) binds to NR2B and NR2D with substantially lower binding to NR2A and NR2C.

The compounds as described herein may bind to NMDA receptors. A disclosed compound may bind to the NMDA receptor resulting in agonist-like activity (facilitation) over a certain dosing range and/or may bind to the NMDA receptor resulting in antagonist-like activity (inhibition) over a certain dosing range. In some embodiments, a disclosed compound may possess a potency that is 10-fold or greater than the activity of existing NMDA receptor modulators.

The disclosed compounds may exhibit a high therapeutic index. The therapeutic index, as used herein, refers to the ratio of the dose that produces a toxicity in 50% of the population (i.e., TD₅₀) to the minimum effective dose for 50% of the population (i.e., ED₅₀). Thus, the therapeutic index=(TD₅₀):(ED₅₀). In some embodiments, a disclosed compound may have a therapeutic index of at least about 10:1, at least about 50:1, at least about 100:1, at least about 200:1, at least about 500:1, or at least about 1000:1.

### Compositions

In other aspects of this disclosure, a pharmaceutical formulation or a pharmaceutical composition including a disclosed compound and a pharmaceutically acceptable excipient are provided. In some embodiments, a pharmaceutical composition includes a racemic mixture or a varied stereoisomeric mixture of one or more of the disclosed compounds.

A formulation can be prepared in any of a variety of forms for use such as for administering an active agent to a patient, who may be in need thereof, as are known in the pharmaceutical arts. For example, the pharmaceutical compositions of the present disclosure can be formulated for administration in solid or liquid form, including those adapted for the following: (1) oral administration, for example, drenches (aqueous or non-aqueous solutions or suspensions), tablets (e.g., those targeted for buccal, sublingual, and/or systemic absorption), boluses, powders, granules, and pastes for application to the tongue; (2) parenteral administration by, for example, subcutaneous, intramuscular, intraperitoneal, intravenous or epidural injection as, for example, a sterile solution or suspension, or sustained-release formulation; (3) topical administration, for example, as a cream, ointment, or a controlled-release patch or spray applied to the skin; (4) intravaginal or intrarectal administration, for example, as a pessary, cream or foam; (5) sublingual administration; (6) ocular administration; (7) transdermal administration; or (8) nasal administration.

For example, pharmaceutical compositions of the disclosure can be suitable for delivery to the eye, i.e., ocularly. Related methods can include administering a therapeutically effective amount of a disclosed compound or a pharmaceutical composition including a disclosed compound to a patient in need thereof, for example, to an eye of the patient, where administering can be topically, subconjunctivally, subtenonly, intravitreally, retrobulbarly, peribulbarly, intracomerally, and/or systemically.

Amounts of a disclosed compound as described herein in a formulation may vary according to factors such as the disease state, age, sex, and weight of the individual. Dosage regimens may be adjusted to provide the optimum therapeutic response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier.

The specification for the dosage unit forms are dictated by and directly dependent on (a) the unique characteristics of the compound selected and the particular therapeutic effect to be achieved, and (b) the limitations inherent in the art of compounding such an active compound for the treatment of sensitivity in individuals.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin.

The compounds can be administered in a time release formulation, for example in a composition which includes a slow release polymer. The compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are generally known to those skilled in the art.

Sterile injectable solutions can be prepared by incorporating the compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

In accordance with an alternative aspect, a compound may be formulated with one or more additional compounds that enhance the solubility of the compound.

### Methods

Methods of the disclosure for treating a condition in a patient in need thereof generally include administering a therapeutically effective amount of a compound described herein or a composition including such a compound. In some embodiments, the condition may be a mental condition. For example, a mental illness may be treated. In some embodiments, a nervous system condition may be treated. For example, a condition that affects the central nervous system, the peripheral nervous system, and/or the eye may be treated. In some embodiments, neurodegenerative diseases may be treated.

In some embodiments, the methods include administering a compound to treat patients suffering from autism, anxiety, depression, bipolar disorder, attention deficit disorder, attention deficit hyperactivity disorder (ADHD), schizophrenia, a psychotic disorder, a psychotic symptom, social withdrawal, obsessive-compulsive disorder (OCD), phobia, post-traumatic stress syndrome, a behavior disorder, an impulse control disorder, a substance abuse disorder (e.g., a withdrawal symptom, opiate addiction, nicotine addiction, and ethanol addition), a sleep disorder, a memory disorder (e.g., a deficit, loss, or reduced ability to make new memories), a learning disorder, urinary incontinence, multiple system atrophy, progressive supra-nuclear palsy, Friedrich's ataxia, Down's syndrome, fragile X syndrome, tuberous sclerosis, olivio-ponto-cerebellar atrophy, cerebral palsy, drug-induced optic neuritis, ischemic retinopathy, diabetic retinopathy, glaucoma, dementia, AIDS dementia, Alzheimer's disease, Huntington's chorea, spasticity, myoclonus, muscle spasm, infantile spasm, Tourette's syndrome, epilepsy, cerebral ischemia, stroke, a brain tumor, traumatic brain injury, cardiac arrest, myelopathy, spinal cord injury, peripheral neuropathy, acute neuropathic pain, and chronic neuropathic pain.

In some embodiments, the present disclosure provides methods of treating a cognitive impairment disorder, for example, a dysfunction in learning and/or memory such as that seen in age-related cognitive decline, Lewy body dementia, AIDS dementia, HIV dementia, vascular dementia, mild cognitive impairment in Huntington's disease, Huntington's disease dementia, mild cognitive impairment in Parkinson's disease, Parkinson's disease dementia, mild cognitive impairment in Alzheimer's disease, Alzheimer's dementia, frontotemporal dementia, cognitive impairment associated with schizophrenia (CIAS), and cognitive impairment associated with seizures, stroke, cerebral ischemia, hypoglycemia, cardiac arrest, migraine, multiple sclerosis, traumatic brain injury, and/or Down's syndrome.

In certain embodiments, methods for treating schizophrenia are provided. For example, paranoid type schizophrenia, disorganized type schizophrenia (i.e., hebephrenic schizophrenia), catatonic type schizophrenia, undifferentiated type schizophrenia, residual type schizophrenia, post-schizophrenic depression, and simple schizophrenia may be treated using the methods and compositions disclosed herein. Psychotic disorders such as schizoaffective disorders, delusional disorders, brief psychotic disorders, shared psychotic disorders, and psychotic disorders with delusions or hallucinations may also be treated using the compositions disclosed herein.

Paranoid schizophrenia may be characterized where delusions or auditory hallucinations are present, but thought disorder, disorganized behavior, or affective flattening are not. Delusions may be persecutory and/or grandiose, but in addition to these, other themes such as jealousy, religiosity, or somatization may also be present. Disorganized type schizophrenia may be characterized where thought disorder and flat affect are present together. Catatonic type schizophrenia may be characterized where the patient may be almost immobile or exhibit agitated, purposeless movement. Symptoms can include catatonic stupor and waxy flexibility. Undifferentiated type schizophrenia may be characterized where psychotic symptoms are present but the criteria for paranoid, disorganized, or catatonic types have not been met. Residual type schizophrenia may be characterized where positive symptoms are present at a low intensity only. Post-schizophrenic depression may be characterized where a depressive episode arises in the aftermath of a schizophrenic illness where some low-level schizophrenic symptoms may still be present. Simple schizophrenia may be characterized by insidious and progressive development of prominent negative symptoms with no history of psychotic episodes.

In some embodiments, methods are provided for treating psychotic symptoms that may be present in other mental disorders, including, but not limited to, bipolar disorder, borderline personality disorder, drug intoxication, and drug-induced psychosis. In another embodiment, methods for treating delusions (e.g., “non-bizarre”) that may be present in, for example, delusional disorder are provided.

Also provided are methods for treating social withdrawal in conditions including, but not limited to, social anxiety disorder, avoidant personality disorder, and schizotypal personality disorder.

In some embodiments, the disclosure provides methods for treating a neurodevelopmental disorder related to synaptic dysfunction in a patient in need thereof, where the methods generally include administering to the patient a therapeutically effective amount of a disclosed compound, or a pharmaceutical composition including a disclosed compound. In certain embodiments, the neurodevelopmental disorder related to synaptic dysfunction can be Rett syndrome also known as cerebroatrophic hyperammonemia, MECP2 duplication syndrome (e.g., a MECP2 disorder), CDKL5 syndrome, fragile X syndrome (e.g., a FMR1 disorder), tuberous sclerosis (e.g., a TSC1 disorder and/or a TSC2 disorder), neurofibromatosis (e.g., a NF1 disorder), Angelman syndrome (e.g., a UBE3A disorder), the PTEN hamartoma tumor syndrome, Phelan-McDermid syndrome (e.g., a SHANK3 disorder), or infantile spasms. In particular embodiments, the neurodevelopmental disorder can be caused by mutations in the neuroligin (e.g., a NLGN3 disorder and/or a NLGN2 disorder) and/or the neurexin (e.g., a NRXN1 disorder).

In some embodiments, methods are provided for treating neuropathic pain. The neuropathic pain may be acute or chronic. In some cases, the neuropathic pain may be associated with a condition such as herpes, HIV, traumatic nerve injury, stroke, post-ischemia, chronic back pain, post-herpetic neuralgia, fibromyalgia, reflex sympathetic dystrophy, complex regional pain syndrome, spinal cord injury, sciatica, phantom limb pain, diabetic neuropathy such as diabetic peripheral neuropathy (“DPN”), and cancer chemotherapeutic-induced neuropathic pain. Methods for enhancing pain relief and for providing analgesia to a patient are also provided.

Further disclosed methods include a method of treating autism and/or an autism spectrum disorder in a patient need thereof, comprising administering an effective amount of a compound to the patient. In some embodiments, a method for reducing the symptoms of autism in a patient in need thereof is provided, comprising administering an effective amount of a disclosed compound to the patient. For example, upon administration, the compound may decrease the incidence of one or more symptoms of autism such as eye contact avoidance, failure to socialize, attention deficit, poor mood, hyperactivity, abnormal sound sensitivity, inappropriate speech, disrupted sleep, and perseveration. Such decreased incidence may be measured relative to the incidence in the untreated individual or an untreated individual(s).

Also provided herein is a method of modulating an autism target gene expression in a cell comprising contacting a cell with an effective amount of a compound described herein. The autism gene expression may be for example, selected from ABAT, APOE, CHRNA4, GABRA5, GFAP, GRIN2A, PDYN, and PENK. In another embodiment, a method of modulating synaptic plasticity in a patient suffering from a synaptic plasticity related disorder is provided, comprising administering to the patient an effective amount of a compound.

In some embodiments, a method of treating Alzheimer's disease, or e.g., treatment of memory loss that e.g., accompanies early stage Alzheimer's disease, in a patient in need thereof is provided, comprising administering a compound. Also provided herein is a method of modulating an Alzheimer's amyloid protein (e.g., beta amyloid peptide, e.g. the isoform Aβ₁₋₄₂), in-vitro or in-vivo (e.g. in a cell) comprising contacting the protein with an effective amount of a compound is disclosed. For example, in some embodiments, a compound may block the ability of such amyloid protein to inhibit long-term potentiation in hippocampal slices as well as apoptotic neuronal cell death. In some embodiments, a disclosed compound may provide neuroprotective properties to a Alzheimer's patient in need thereof, for example, may provide a therapeutic effect on later stage Alzheimer's-associated neuronal cell death.

In certain embodiments, the disclosed methods include treating a psychosis or a pseudobulbar affect (“PBA”) that is induced by another condition such as a stroke, amyotrophic lateral sclerosis (ALS or Lou Gehrig's disease), multiple sclerosis, traumatic brain injury, Alzheimer's disease, dementia, and/or Parkinson's disease. Such methods, as with other methods of the disclosure, include administration of a therapeutically effective amount of a disclosed compound to a patient in need thereof.

In various embodiments, a method of treating depression comprising administering a compound described herein is provided. In some embodiments, the treatment may relieve depression or a symptom of depression without affecting behavior or motor coordination and without inducing or promoting seizure activity. Exemplary depression conditions that are expected to be treated according to this aspect include, but are not limited to, major depressive disorder, dysthymic disorder, psychotic depression, postpartum depression, premenstrual syndrome, premenstrual dysphoric disorder, seasonal affective disorder (SAD), bipolar disorder (or manic depressive disorder), mood disorder, and depressions caused by chronic medical conditions such as cancer or chronic pain, chemotherapy, chronic stress, and post traumatic stress disorders. In addition, patients suffering from any form of depression often experience anxiety. Various symptoms associated with anxiety include fear, panic, heart palpitations, shortness of breath, fatigue, nausea, and headaches among others. Anxiety or any of the symptoms thereof may be treated by administering a compound as described herein.

Also provided herein are methods of treating a condition in treatment-resistant patients, e.g., patients suffering from a mental or central nervous system condition that does not, and/or has not, responded to adequate courses of at least one, or at least two, other compounds or therapeutics. For example, provided herein is a method of treating depression in a treatment resistant patient, comprising a) optionally identifying the patient as treatment resistant and b) administering an effective dose of a compound to said patient.

In some embodiments, a compound described herein may be used for acute care of a patient. For example, a compound may be administered to a patient to treat a particular episode (e.g., a severe episode) of a condition described herein.

Also provided herein are combination therapies comprising a compound in combination with one or more other active agents. For example, a compound may be combined with one or more antidepressants, such as tricyclic antidepressants, MAO-I's, SSRI's, and double and triple uptake inhibitors and/or anxiolytic drugs. Exemplary drugs that may be used in combination with a compound include Anafranil, Adapin, Aventyl, Elavil, Norpramin, Pamelor, Pertofrane, Sinequan, Surmontil, Tofranil, Vivactil, Parnate, Nardil, Marplan, Celexa, Lexapro, Luvox, Paxil, Prozac, Zoloft, Wellbutrin, Effexor, Remeron, Cymbalta, Desyrel (trazodone), and Ludiomill. In another example, a compound may be combined with an antipsychotic medication. Non-limiting examples of antipsychotics include butyrophenones, phenothiazines, thioxanthenes, clozapine, olanzapine, risperidone, quetiapine, ziprasidone, amisulpride, asenapine, paliperidone, iloperidone, zotepine, sertindole, lurasidone, and aripiprazole. It should be understood that combinations of a compound and one or more of the above therapeutics may be used for treatment of any suitable condition and are not limited to use as antidepressants or antipsychotics.

### EXAMPLES

The following examples are provided for illustrative purposes only, and are not intended to limit the scope of the disclosure.

The compounds described herein can be prepared in a number of ways based on the teachings contained herein and synthetic procedures known in the art. In the description of the synthetic methods described below, it is to be understood that all proposed reaction conditions, including choice of solvent, reaction atmosphere, reaction temperature, duration of the experiment and workup procedures, can be chosen to be the conditions standard for that reaction, unless otherwise indicated. It is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated. The starting materials for the examples are either commercially available or are readily prepared by standard methods from known materials. At least some of the compounds identified as “intermediates” herein can be compounds of the disclosure.

The following abbreviations may be used herein and have the indicated definitions: Ac is acetyl (—C(O)CH₃), ACN is acetonitrile, AIDS is acquired immune deficiency syndrome, Boc and BOC are tert-butoxycarbonyl, Boc₂O is di-tert-butyl dicarbonate, Bn is benzyl, Cbz is carboxybenzyl, DCM is dichloromethane, DEA is diethylamine, DIPA is diisopropylamine, DIPEA is N,N-diisopropylethylamine, DMF is N,N-dimethylformamide, DMSO is dimethyl sulfoxide, ESI is electrospray ionization, EtOAc is ethyl acetate, h is hour, HATU is 2-(7-aza-1H-benzotriazole-1-yl)-1,1,3,3-tetramethyluronium hexafluorophosphate, HIV is human immunodeficiency virus, HPLC is high performance liquid chromatography, LCMS is liquid chromatography/mass spectrometry, LDA is lithium diisopropylamide, LiHMDS is lithium hexamethyldisilazane, Ms is mesyl or methanesulfonyl, NMDAR is N-methyl-d-aspartate receptor, NMR is nuclear magnetic resonance, Pd/C is palladium on carbon, PPA is polyphosphoric acid, RT is room temperature (e.g., from about 20° C. to about 25° C.), SM is starting material, TEA is triethylamine, TLC is thin layer chromatography, TFA is trifluoroacetic acid, THF is tetrahydrofuran, TMS is trimethylsilyl, and Ts is tosyl or para-toluenesulfonyl.

### A. Synthesis of Compounds

### Synthesis of AK and AL

An exemplary synthesis of a compound disclosed herein is outlined in Scheme 1, shown below. For example, treatment of aziridine-2-carboxylic acid (SM1) with thionyl chloride in methanol provides methyl ester Int-1 as its hydrochloride salt. Treatment of Int-1 with (Boc)₂O under neutralizing conditions (e.g., in the presence of Et₃N) affords Boc-protected Int-2. Treatment of Int-2 with paraformaldehyde and LiHMDS gives spirolactam Int-3. Removal of the Boc protecting group in Int-3 under acidic conditions (e.g., in the presence of TFA) furnishes Examples AK and AL.

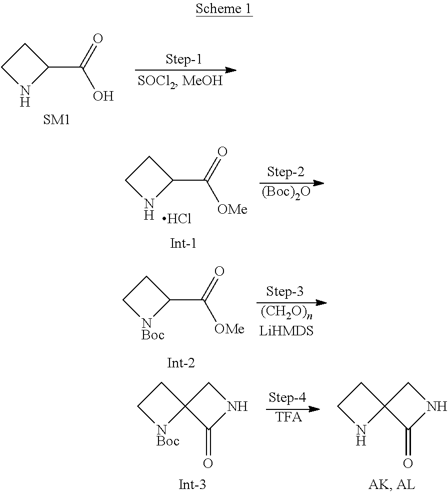

### Synthesis of AU-2

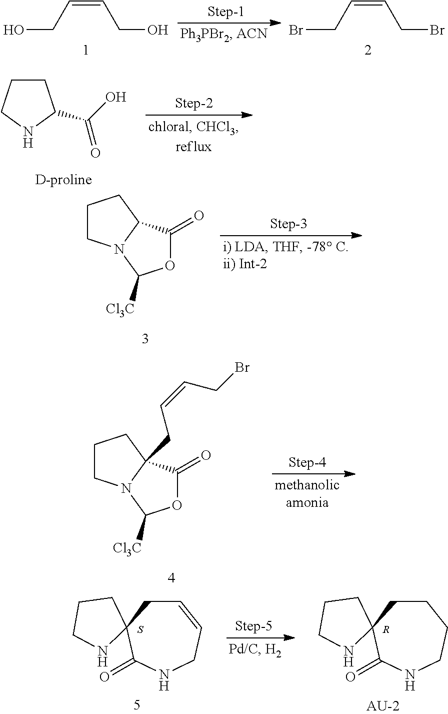

### Synthesis of (Z)-1,4-dibromobut-2-ene (2)

To a stirred solution of triphenylphosphine (100 g, 0.381 mol) in ACN (500 mL), bromine (19 mL, 0.381 mol) was added dropwise at 0° C. and stirred at same temperature for 1 h. After that (Z)-but-2-ene-1,4-diol (15 g) was added and reaction mixture was heated at 50° C. for 4 h. After consumption of the starting material (by TLC), the reaction mixture was quenched with water (300 mL) and extracted with Et₂O (3×300 mL). The combined organic layer was washed with brine (100 mL), dried over Na₂SO₄ and concentrated under reduced pressure to afford 2 (26 g, crude) as thick oil. ¹H NMR (400 MHz, DMSO-d₆) δ 6.03-5.86 (m, 2H), 4.06-3.95 (m, 4H).

### Synthesis of (3S,7aR)-3-(trichloromethyl)tetrahydro-1H,3H-pyrrolo[1,2-c]oxazol-1-one (3)

To a stirring solution of D-Proline (5 g, 43.4 mmol) in chloroform (100 mL), chloral (8.5 g, 52.1 mmol) was added and reaction mixture was heated at 65° C. for 16 h (using dean-stark apparatus). After consumption of the starting material (by TLC), the reaction mixture was concentrated under reduced pressure. Recrystallization in ethanol afforded compound 3 (4 g, 38%) as a white solid. ¹H NMR (400 MHz, DMSO-d₆): δ 5.16 (s, 1H), 4.18-4.10 (m, 1H), 3.45-3.39 (m, 1H), 3.15-3.09 (m, 1H), 2.27-2.18 (m, 1H), 2.12-2.08 (m, 1H), 1.97-1.92 (m, 1H), 1.79-1.73 (m, 1H).

### Synthesis of (3S,7aS)-7a-((Z)-4-bromobut-2-en-1-yl)-3-(trichloromethyl)tetrahydro-1H,3H-pyrrolo[1,2-c]oxazol-1-one (4)

To a stirred solution of compound 3 (3.7 g, 15.13 mmol) in THF (40 mL), LDA (2M solution in THF, 22.6 mL, 22.6 mmol) was added at −78° C. and stirred at same temperature for 20 min. To the reaction mixture, compound A (4.7 g, 22.6 mmol) was added dropwise at −78° C. and stirred at same temperature for 4 h. After consumption of the starting material (by TLC), the reaction mixture was quenched with water (300 mL) and extracted with EtOAc (3×200 mL). The combined organic layer was washed with brine (100 mL), dried over Na₂SO₄ and concentrated under reduced pressure. The residue was purified by flash column chromatography to afford compound 4 (3.8 g, 67.8%) as thick oil. LCMS (ESI): m/z 376 [M+1].

### Synthesis of (S)-1,7-diazaspiro[4.6]undec-9-en-6-one (5)

To a stirred solution of compound 4 (3.5 g, 9.35 mmol) in MeOH (20 mL), methanolic ammonia (20 mL) was added at 0° C. under nitrogen atmosphere and stirred at room temperature for 16 h. After consumption of the starting material (by TLC), and then evaporated to give a residue which was dissolved in 2 M HCl. The acidic layer was washed with ethyl acetate and then made basic (pH 12) by the addition of solid NaOH. Extraction with dichloromethane and dried over Na₂SO₄ and concentrated under reduced pressure. The residue was purified by column chromatography to afford compound 5 (0.3 g, 20%) as a pale yellow semisolid. LCMS (ESI): m/z 167 [M+1].

### Synthesis of (R)-1,7-diazaspiro[4.6]undecan-6-one (AU-2

To a stirring solution of compound 5 (0.15 g, 0.9 mmol) in MeOH (2 mL) and EtOAc (2 mL), 10% Pd/C (20 mg) was added at room temperature and stirred under H₂ atmosphere (balloon) for 4 h. After consumption of the starting material (by TLC), the reaction mixture was filtered through a pad of celite and washed with MeOH (50 mL). The filtrate was concentrated under reduced pressure. The residue was purified by flash chromatography to afford compound AU-2 (120 mg, 80%) as off white solid. ¹H NMR (400 MHz, DMSO-d₆): δ 7.89 (brs, 1H), 3.10-3.03 (m, 2H), 3.02-2.99 (m, 1H), 2.86-2.80 (m, 1H), 2.08-2.01 (m, 1H), 1.93-1.90 (m, 1H), 1.81-1.54 (m, 8H), 1.40-1.23 (m, 1H). LCMS (ESI): m/z 169 [M+1]. HPLC: 95.08%.

### Synthesis of EI-1 & EI-2

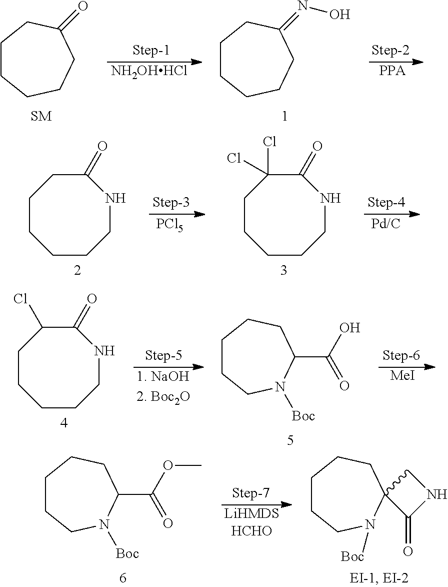

### Synthesis of Cycloheptanone Oxime (1)

To a stirred solution of cycloheptanone (SM) (20 g, 178.3 mmol) in ethanol (200 mL) was added hydroxylamine hydrochloride (14.9 g, 213.9 mmol) and then heated to reflux for 1 h. After consumption of the starting material (by TLC), the reaction mixture was brought to RT and volatiles were evaporated under reduced pressure. Crude material was diluted with water (200 mL) and extracted with EtOAc (2×200 mL). Combined organic layer was dried over Na₂SO₄ and concentrated under reduced pressure to obtain compound 1 (15.5 g, 68%) as off white solid, which was taken next step without any further purification. ¹H-NMR: (500 MHz, DMSO-d₆): δ 10.24 (br s, 1H), 2.40 (t, J=5.5 Hz, 2H), 2.28 (t, J=5.5 Hz, 2H), 1.60-1.40 (m, 8H). LCMS (m/z): 128 [M⁺+1].

### Synthesis of azocan-2-one (2)

To a solution of compound 1 (10.5 g, 82.5 mmol) in o-xylene (63 mL) was added polyphosphoric acid (15 mL). The reaction mixture was heated to 120° C. and stirred for 1 h. After consumption of the starting material (by TLC), the reaction mixture was brought to RT and o-xylene was removed by decantation. Crude material was diluted with cold water (20 mL) and extracted with DCM (3×100 mL). Combined organic layer was dried over Na₂SO₄ and concentrated under reduced pressure to obtain compound 2 (9.5 g, 90%) as reddish brown thick syrup, which was taken next step without any further purification. ¹H-NMR: (400 MHz, DMSO-d₆): δ 7.12 (d, J=3.6 Hz, 1H), 3.19-3.15 (m, 2H), 2.26-2.20 (m, 2H), 1.62-1.59 (m, 2H), 1.51-1.43 (m, 6H). LCMS (ESI): m/z 128.1 [M⁺+1].

### Synthesis of 3,3-dichloroazocan-2-one (3)

To a solution of compound 2 (9.5 g, 74.6 mmol) in DCM (19 mL) were added toluene (76 mL) and PCl₅ (31.1 g, 149.3 mmol) at RT under nitrogen atmosphere. The reaction mixture was heated to reflux and stirred for 2 h. After consumption of the starting material (by TLC), the reaction mixture was brought to RT and volatiles were evaporated under reduced pressure. Crude material was diluted with ice water (50 mL) and acetone (30 mL). Aqueous NaHCO₃ solution was added and pH was adjusted to 8 and then reaction mixture was extracted with DCM (2×100 mL). Combined organic layer was dried over Na₂SO₄ and concentrated under reduced pressure. Obtained crude material was purified by silica gel column chromatography eluting 20% EtOAc/hexane to afford compound 3 (6.7 g, 46%) as white solid. ¹H-NMR: (500 MHz, DMSO-d₆): δ 7.92 (s, 1H), 3.41 (br s, 2H), 2.78 (s, 2H), 1.70-1.60 (m, 4H), 1.42-1.23 (m, 2H). LCMS (ESI): m/z 196.1 [M⁺+1].

### Synthesis of 3-chloroazocan-2-one (4)

To a stirring solution of compound 3 (2.6 g, 13.2 mmol) in methanol (39 mL) were added acetic acid (7.8 mL), sodium acetate (3 g, 36.5 mmol) and 10% Pd/C (650 mg) at RT under nitrogen atmosphere. The reaction mixture was stirred at RT for 2 h under H₂ atmosphere. After consumption of the starting material (by TLC), the reaction mixture was filtered through a pad of celite and volatiles were evaporated under reduced pressure. Aqueous NaHCO₃ solution was added and pH was adjusted to 8 and then reaction mixture was extracted with DCM (2×50 mL). Combined organic layer was dried over Na₂SO₄ and concentrated under reduced pressure to obtain compound 4 (2.1 g, crude) as white solid, which was taken next step without any further purification. ¹H-NMR: (500 MHz, DMSO-d₆): δ 7.68 (s, 1H), 5.15-5.12 (m, 1H), 3.51-3.44 (m, 1H), 3.08-3.04 (m, 1H), 2.07-2.01 (m, 1H), 1.88-1.81 (m, 1H), 1.68-1.62 (m, 4H), 1.48-1.40 (m, 2H). LCMS (ESI): m/z 162.1 [M⁺+1].

### Synthesis of 1-(tert-butoxycarbonyl)azepane-2-carboxylic acid (5)

To a stirring solution of compound 4 (1.6 g, 9.9 mmol) in 1,4-dioxane (16 mL) was added NaOH (3.56 g, 89.1 mmol) and then heated to reflux for 16 h. The reaction mixture was cooled to 0° C., added water (8 mL) and Boc-anhydride (4.3 mL, 19.8 mmol) and allowed to stir for 5 h. After consumption of the starting material (by TLC), the reaction was diluted with water (10 mL) and extracted with CH₂Cl₂ (1×10 mL). Aqueous layer pH was adjusted to 2 using 2N HCl and then reaction mixture was extracted with DCM (2×50 mL). The combined organic layer was dried over Na₂SO₄ and concentrated under reduced pressure to afford crude compound 5 (1.49 g, crude) as colorless thick syrup, which was taken next step without any further purification. ¹H-NMR: (500 MHz, DMSO-d₆): δ 12.56 (br s, 1H), 4.35-4.32 (m, 1H), 3.74-3.64 (m, 2H), 2.98-2.87 (m, 2H), 2.24-2.12 (m, 2H), 1.46-1.34 (m, 4H), 1.34 (s, 9H). LCMS (ESI): m/z 241.8 [M⁺−1].

### Synthesis of 1-(tert-butyl) 2-methyl azepane-1,2-dicarboxylate (6)

To a stirring solution of compound 5 (1.4 g, 5.7 mmol) in acetonitrile (14 mL) were added K₂CO₃ (2.38 g, 17.2 mmol) and Mel (0.72 mL, 11.5 mmol) at 0° C. under nitrogen atmosphere. The reaction mixture was brought to RT and allowed to stir for 16 h. After consumption of the starting material (by TLC), the reaction was diluted with water (20 mL) and extracted with EtOAc (2×30 mL). Combined organic layer was dried over Na₂SO₄ and concentrated under reduced pressure. Obtained crude material was purified by silica gel column chromatography eluting 10% EtOAc/n-hexane to afford compound 6 (720 mg, 49%) as colorless thick syrup. ¹H-NMR: (500 MHz, DMSO-d₆): δ 4.47-4.44 (m, 1H), 3.62 (s, 3H), 3.06-2.91 (m, 2H), 2.21-2.08 (m, 2H), 1.76-1.60 (m, 6H), 1.33 (s, 9H). LCMS (ESI): m/z 158.2 [(M⁺+1)-Boc].

### Synthesis of tert-butyl 1-oxo-2,5-diazaspiro[3.6]decane-5-carboxylate (EI-1 & EI-2)

To a stirring solution of compound 6 (760 mg, 2.9 mmol) in THF (7.6 mL) was added paraformaldehyde (106 mg, 3.5 mmol) at RT under nitrogen atmosphere. The reaction mixture was cooled to −78° C. and added LiHMDS (8.8 mL, 8.8 mmol) and allowed to stir at RT for 4 h. After consumption of the starting material (by TLC), the reaction was quenched with water (10 mL) and extracted with EtOAc (2×20 mL). The combined organic layer was washed with water (2×15 mL) followed by brine solution (2×10 mL). The organic layer was dried over Na₂SO₄ and concentrated to obtain crude material which was purified by column chromatography by eluting 40% EtOAC/n-hexane to afford racemic EI-1 and EI-2 (450 mg, 60%) as white solid. The racemic was separated by chiral HPLC purification and obtained 150 mg of EI-1 and 160 mg of EI-2.

EI-1: ¹H-NMR: (400 MHz, DMSO-d₆): δ 7.82 (s, 1H), 3.67-3.61 (m, 1H), 3.34-3.26 (m, 2H), 3.06 (d, J=5.6 Hz, 1H), 2.20-2.13 (m, 1H), 1.98-1.95 (m, 1H), 1.78-1.54 (m, 4H), 1.40-1.38 (m, 1H), 1.39 (s, 9H), 1.29-1.21 (m, 1H). LCMS (ESI): m/z 153.1 [(M⁺+1)-Boc]. HPLC: 99.72%.

EI-2: ¹H-NMR: (400 MHz, DMSO-d₆): δ 7.82 (s, 1H), 3.67-3.61 (m, 1H), 3.34-3.24 (m, 2H), 3.06 (d, J=5.6 Hz, 1H), 2.20-2.13 (m, 1H), 1.98-1.95 (m, 1H), 1.78-1.54 (m, 4H), 1.40-1.38 (m, 1H), 1.39 (s, 9H), 1.28-1.21 (m, 1H). LCMS (ESI): m/z 153.1 [(M⁺+1)-Boc]. HPLC: 99.77%.

Following the above procedures, the following compounds and stereoisomers thereof were or are prepared. It will be appreciated by a person of skill in the art that for structures shown additional diastereomers and/or enantiomers may be envisioned and are included herein.

TABLE 1

| Com- | |
|---|---|
| pound | Structure |
| AA, AB | 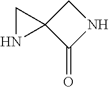 |
| AC, AD | 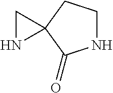 |
| AE, AF | 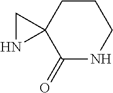 |
| AG, AH | 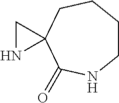 |
| AI, AJ | 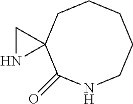 [SMILES: O=C1NCCCCCC12CN2] |
| AK, AL | 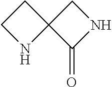 |
| AM, AN | 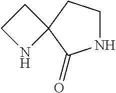 |
| AO, AP | 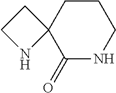 |
| AQ, AR | 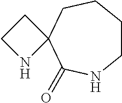 [SMILES: O=C1NCCCCC12CCN2] |
| AS, AT | 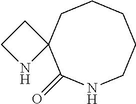 [SMILES: O=C1NCCCCCC12CCN2] |
| AU-1, AU-2 | 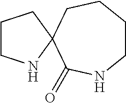 [SMILES: O=C1NCCCCC12CCCN2] |
| AV, AW | 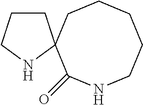 [SMILES: O=C1NCCCCCC12CCCN2] |
| AX, AY | 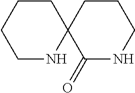 [SMILES: O=C1NCCCC12CCCCN2] |
| AZ, BA | 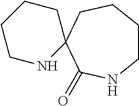 [SMILES: O=C1NCCCCC12CCCCN2] |
| BB, BC | 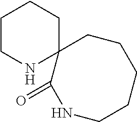 [SMILES: O=C1NCCCCCC12CCCCN2] |
| BD, BE | 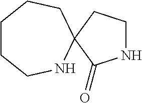 [SMILES: O=C1NCCC12CCCCCN2] |
| BF, BG | 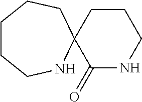 [SMILES: O=C1NCCCC12CCCCCN2] |
| BH, BI | 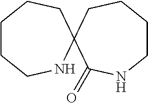 [SMILES: O=C1NCCCCC12CCCCCN2] |
| BJ, BK | 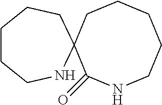 [SMILES: O=C1NCCCCCC12CCCCCN2] |
| BL, BM | 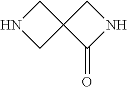 |
| BN, BO | 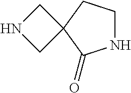 |
| BP, BQ | 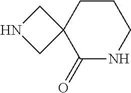 |
| BR, BS | 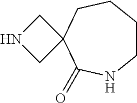 [SMILES: O=C1NCCCCC12CNC2] |
| BT, BU | 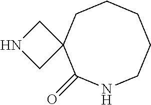 [SMILES: O=C1NCCCCCC12CNC2] |
| BV, BW | 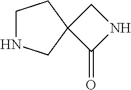 |
| BX, BY | 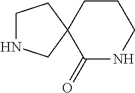 [SMILES: O=C1NCCCC12CCNC2] |
| BZ, CA | 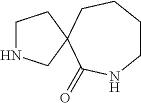 [SMILES: O=C1NCCCCC12CCNC2] |
| CB, CC | 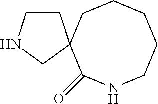 [SMILES: O=C1NCCCCCC12CCNC2] |
| CD, CE | 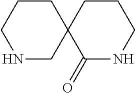 [SMILES: O=C1NCCCC12CCCNC2] |
| CF, CG | 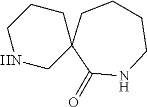 [SMILES: O=C1NCCCCC12CCCNC2] |
| CH, CI | 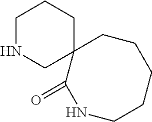 [SMILES: O=C1NCCCCCC12CCCNC2] |
| CP, CQ | 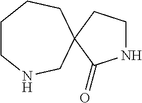 [SMILES: O=C1NCCC12CCCCNC2] |
| CR, CS | 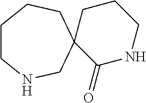 [SMILES: O=C1NCCCC12CCCCNC2] |
| CT, CU | 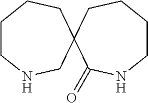 [SMILES: O=C1NCCCCC12CCCCNC2] |
| CV, CW | 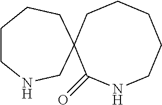 [SMILES: O=C1NCCCCCC12CCCCNC2] |
| CX, CY | 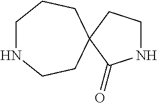 [SMILES: O=C1NCCC12CCCNCC2] |
| CZ, DA | 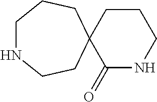 [SMILES: O=C1NCCCC12CCCNCC2] |
| DB, DC | 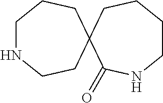 [SMILES: O=C1NCCCCC12CCCNCC2] |
| DD, DE | 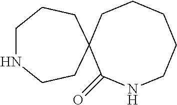 [SMILES: O=C1NCCCCCC12CCCNCC2] |
| DF, DG | 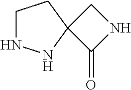 |
| DH, DI | 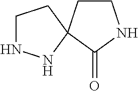 |
| DJ, DK | 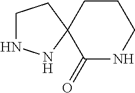 [SMILES: O=C1NCCCC12CCNN2] |
| DL, DM | 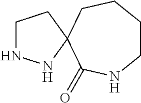 [SMILES: O=C1NCCCCC12CCNN2] |
| DN, DO | 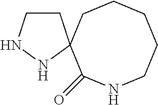 [SMILES: O=C1NCCCCCC12CCNN2] |
| DP-1, DP-2 | 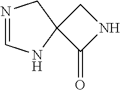 |
| DQ-1, DQ-2 | 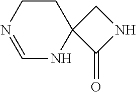 |
| DR-1, DR-2 | 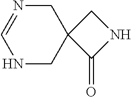 |
| DS-1, DS-2 | 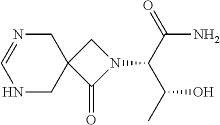 [SMILES: C[C@@H](O)[C@@H](C(N)=O)N1CC2(CN=CNC2)C1=O] |
| DT-1, DT-2 | 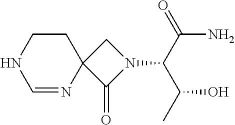 [SMILES: C[C@@H](O)[C@@H](C(N)=O)N1CC2(CCNC=N2)C1=O] |
| DU-1, DU-2 | 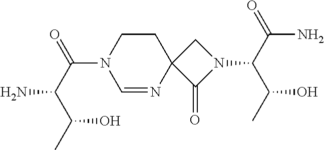 [SMILES: C[C@@H](O)[C@H](N)C(=O)N1C=NC2(CC1)CN([C@H](C(N)=O)[C@@H](C)O)C2=O] |
| DV-1, DV-2 | 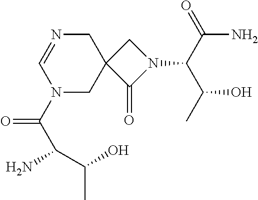 [SMILES: C[C@@H](O)[C@H](N)C(=O)N1C=NCC2(C1)CN([C@H](C(N)=O)[C@@H](C)O)C2=O] |
| DW-1, DW-2 | 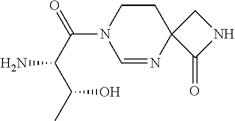 [SMILES: C[C@@H](O)[C@H](N)C(=O)N1C=NC2(CC1)CNC2=O] |
| DX-1, DX-2 | 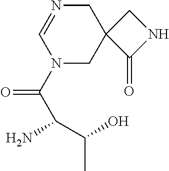 [SMILES: C[C@@H](O)[C@H](N)C(=O)N1C=NCC2(CNC2=O)C1] |
| DY-1, DY-2 | 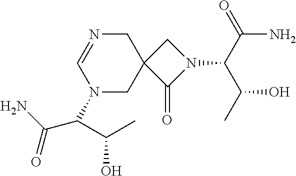 |
| DZ-1, DZ-2 | 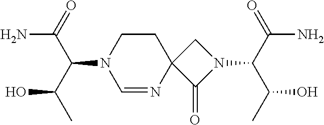 [SMILES: C[C@@H](O)[C@@H](C(N)=O)N1C=NC2(CC1)CN([C@H](C(N)=O)[C@@H](C)O)C2=O] |
| EA-1, EA-2 | 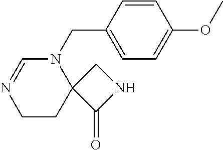 [SMILES: COc1ccc(CN2C=NCCC23CNC3=O)cc1] |
| EB-1, EB-2 | 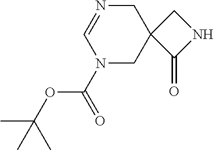 [SMILES: CC(C)(C)OC(=O)N1C=NCC2(CNC2=O)C1] |
| EC-1, EC-2 | 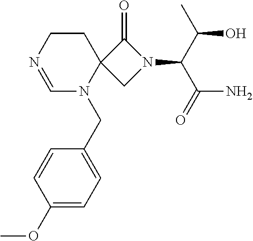 [SMILES: COc1ccc(CN2C=NCCC23CN([C@H](C(N)=O)[C@@H](C)O)C3=O)cc1] |
| EF-1, EF-2 | 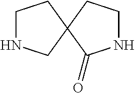 |
| EI-1, EI-2 | 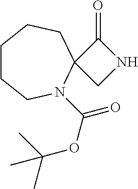 [SMILES: CC(C)(C)OC(=O)N1CCCCCC12CNC2=O] |
| EU-1, EU-2 | 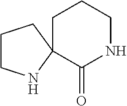 [SMILES: O=C1NCCCC12CCCN2] |
| EV-1, EV-2 | 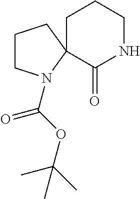 [SMILES: CC(C)(C)OC(=O)N1CCCC12CCCNC2=O] |

### Synthesis of GA

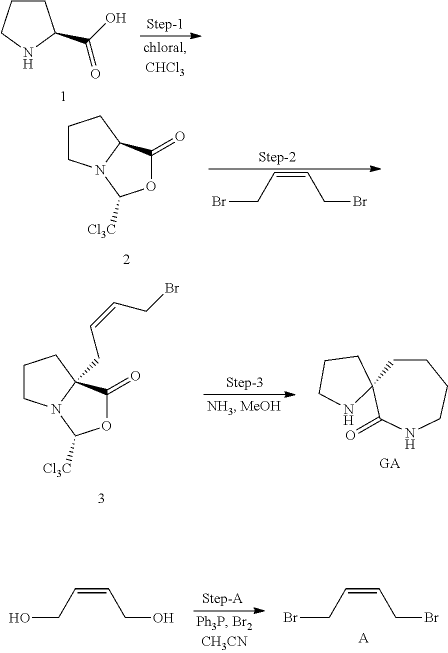

### Synthesis of (Z)-1,4-dibromobut-2-ene (A)

To a stirred solution of compound triphenylphosphane (100 g, 0.381 mol) in ACN (500 mL), bromine (19 mL, 0.381 mol) was added dropwise at 0° C. and stirred at same temperature for 1 h. After that (Z)-but-2-ene-1,4-diol (15 g, 0.381 mol) was added and reaction mixture was heated at 50° C. for 4 h. After consumption of the starting material (by TLC), the reaction mixture was quenched with water (300 mL) and extracted with Et₂O (3×300 mL). The combined organic layer was washed with brine (100 mL), dried over Na₂SO₄ and concentrated under reduced pressure to afford compound A (26 g, crude) as thick oil. ¹H NMR (400 MHz, DMSO-d₆) δ 6.03-5.86 (m, 2H), 4.06-3.95 (m, 4H).

### Synthesis of (3R,7aS)-3-(trichloromethyl)tetrahydro-1H,3H-pyrrolo[1,2-c]oxazol-1-one (2)

To a stirring solution of compound 1 (10.0 g, 91.2 mmol) in chloroform (400 mL), chloral (26.5 g, 109 mmol) was added and reaction mixture was heated at 65° C. for 16 h (using reverse Dean-Stark apparatus). After consumption of the starting material (by TLC), the reaction mixture was concentrated under reduced pressure. The residue was recrystallized with ethanol to afford compound 2 (9.0 g, 42%) as a white solid. ¹H NMR (400 MHz, DMSO-d₆) δ 5.23 (s, 1H), 4.11-4.08 (m, 1H), 3.43-3.37 (m, 1H), 3.13-3.07 (m, 1H), 2.20-2.18 (m, 1H), 2.11-2.08 (m, 1H), 1.92-1.88 (m, 1H), 1.75-1.70 (m, 1H).

### Synthesis of (3R,7aR)-7a-((Z)-4-bromobut-2-en-1-yl)-3-(trichloromethyl)tetrahydro-1H,3H-pyrrolo[1,2-c]oxazol-1-one (3)

To a stirred solution of compound 2 (10.0 g, 40.8 mmol) in THF (125 mL), LDA (2M solution in THF, 30.6 mL, 61.3 mmol) was added at −78° C. and stirred at same temperature for 20 min. Compound A (17.2 g, 81.7 mmol) was added dropwise at −78° C. and stirred at same temperature for 4 h. After consumption of the starting material (by TLC), the reaction mixture was quenched with water (300 mL) and extracted with EtOAc (3×200 mL). The combined organic layer was washed with brine (100 mL), dried over Na₂SO₄ and concentrated under reduced pressure. The residue was purified by flash column chromatography to afford compound 3 (4.5 g, 29%) as thick oil. ¹H NMR (400 MHz, DMSO-d₆) δ 6.01-5.94 (m, 1H), 5.80-5.40 (m, 1H), 5.01 (s, 1H), 4.09-4.04 (m, 1H), 4.0-3.96 (m, 2H), 3.26-3.20 (m, 2H), 2.80-2.59 (m, 2H), 2.26-2.16 (m, 1H), 2.06-1.90 (m, 2H).

### Synthesis of (R)-1,7-diazaspiro[4.6]undec-9-en-6-one (GA)

To a stirred solution of compound 3 (4.5 g, 12.0 mmol) in MeOH (20 mL), methanolic ammonia (70 mL) was added at 0° C. under nitrogen atmosphere and stirred at room temperature for 16 h. After consumption of the starting material (by TLC), and then evaporated to give a residue was dissolved in 2 M HCl. The acidic layer was washed with ethyl acetate and then made basic (pH 12) by the addition of solid NaOH. Extraction with dichloromethane and dried over Na₂SO₄ and concentrated under reduced pressure. The residue was purified by column chromatography to afford compound GA (1.0 g, 52.6%) as a pale yellow solid. ¹H NMR (400 MHz, DMSO-d₆) δ 7.65 (s, 1H), 5.70-5.54 (m, 2H), 3.80-3.59 (m, 2H), 3.26-3.14 (m, 1H), 2.76 (d, J=6.9 Hz, 2H), 2.21-2.00 (m, 3H), 1.78-1.52 (m, 3H). LCMS (ESI): m/z 167 [M+1]. HPLC: 95.4%.

### Synthetic Scheme for GB-1 and GB-2

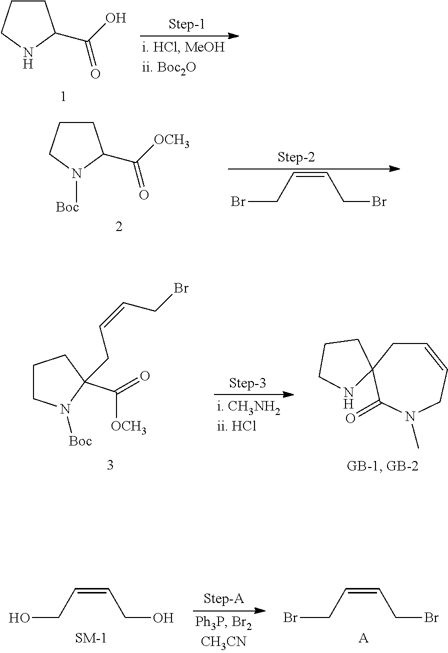

Proline (1) is converted to its corresponding ester and treated with Boc₂O to afford Int-2. Int-2 is lithiated and subjected to alkylation with 1,4-dibromobut-2-ene, which is prepared from dihydroxybut-2-ene, to afford Int-3. Int-3 is cyclized using methylamine followed by treatment with HCl, which products on chiral preparative purification afford GB-1 and GB-2.

### Synthetic Scheme for GF-1 and GF-2

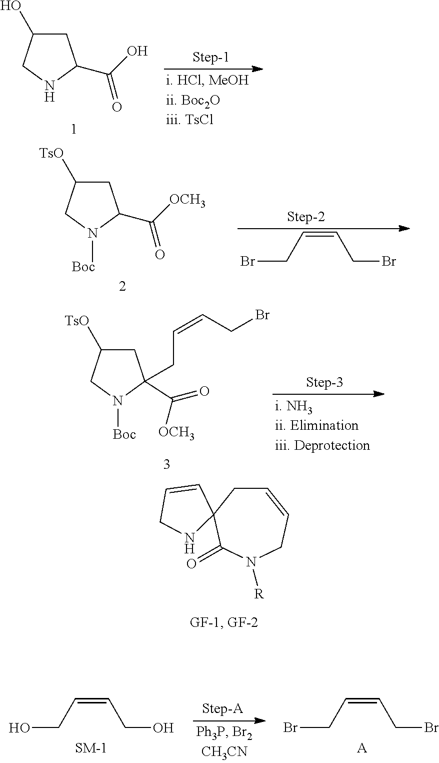

Trans-4-hydroxy-L-proline (1) is esterified and treated with Boc₂O followed by treatment with TsCl, which produces Int-2. Int-2 is alkylated with 1,4-dibromobut-2-ene (which is prepared from dihydroxybut-2-ene) to afford Int-3. Int-3 is cyclized using ammonia followed by elimination, treatment with HCl and preparative purification to afford GF-1 and GF-2.

### Synthetic Scheme for GP-1 and GP-2

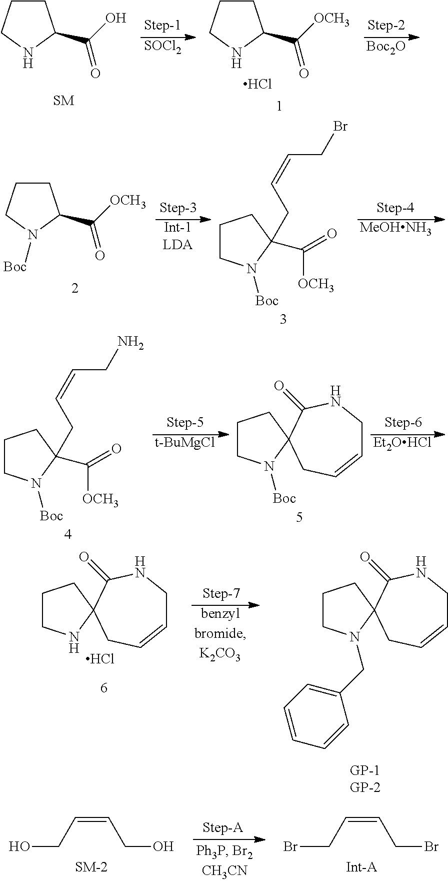

### Synthesis of Methyl L-Prolinate Hydrochloride (1)

To a stirred suspension of L-proline (SM) (200 g, 1.73 mol) in methanol (1 L) was added thionyl chloride (249 mL, 3.47 mol) dropwise at 0° C. under nitrogen atmosphere. The reaction mixture was stirred at 80° C. for 16 h. After consumption of the starting material (by TLC), reaction mixture was brought to RT and volatiles were concentrated under reduced pressure. The crude was triturated with Et₂O and dried under vacuum to afford compound 1 as hydrochloride salt (240 g, 83%) as an off white sticky solid. ¹H NMR (400 MHz, DMSO-d₆) δ 10.50 (brs, 1H), 9.13 (brs, 1H), 4.37-4.33 (m, 1H), 3.75 (s, 3H), 3.26-3.11 (m, 2H), 2.28-2.20 (m, 1H), 2.05-1.83 (m 3H).

### Synthesis of 1-(tert-butyl) 2-methyl (S)-pyrrolidine-1,2-dicarboxylate (2)

To a stirring solution of compound 1 (240 g, 1.44 mol) in CH₂Cl₂ (2.4 L) was added Et₃N (503 mL, 3.62 mol) at 0° C. and stirred for 10 min. Boc₂O (473 mL, 2.17 mol) was added at 0° C. and the reaction mixture was stirred at RT for 16 h. After consumption of the starting material (by TLC), the reaction was quenched with water (1 L) and extracted with CH₂Cl₂ (2×1 L). The combined organic layer was washed with aqueous NH₄Cl solution (1 L), brine (1 L). The organic layer was dried over Na₂SO₄ and concentrated under reduced pressure and the crude was purified by column chromatography by eluting with 20% EtOAc/n-hexane to obtain compound 2 (300 g, 90%) as a thick liquid. ¹H NMR (500 MHz, DMSO-d₆) δ 4.20-4.10 (m, 1H), 3.67-3.61 (m, 3H), 3.36-3.31 (m, 2H), 2.26-2.12 (m, 1H), 1.90-1.76 (m, 3H), 1.39, 1.32 (d, 9H).

### Synthesis of 1-(tert-butyl) 2-methyl (Z)-2-(4-aminobut-2-en-1-yl)pyrrolidine-1,2-dicarboxylate (3)

To a solution of diisopropylamine (36 mL, 0.26 mol) in THF (100 mL) was added n-BuLi (2.5 M in hexane, 104 mL, 0.261 mol) drop wise at −78° C. under nitrogen atmosphere. After completion of addition, temperature of the reaction mixture was raised to −20° C. and stirred for 30 minutes. Again cooled to −78° C., compound 2 (40 g, 0.17 mol) in THF (100 mL) was added dropwise and stirred at −40° C. for 30 minutes. Again cooled to −78° C., Int-A (44.6 g, 0.209 mol) was added to the reaction at −78° C. Reaction mixture was brought to RT and stirred for 3 h. After consumption of the starting material (by TLC), the reaction mixture was quenched with aqueous NH₄Cl (200 mL) and extracted with EtOAc (2×300 mL). The combined organic layer was washed with brine (300 mL), dried over Na₂SO₄ and concentrated under reduced pressure to afford crude compound which was purified by column chromatography by eluting with 20% EtOAc/n-hexane to afford compound 3 (18 g, 28%) as a brown viscous liquid. ¹H NMR (400 MHz, DMSO-d₆) δ 5.94-5.73 (m, 2H), 4.30-4.03 (m, 2H), 3.67 (s, 3H), 3.55-3.40 (m, 2H), 2.87-2.64 (m, 2H), 2.08-1.96 (m, 2H), 1.87-1.70 (m, 2H), 1.38, 1.33 (2s, 9H).

### Synthesis of 1-(tert-butyl) 2-methyl (Z)-2-(4-bromobut-2-en-1-yl)pyrrolidine-1,2-dicarboxylate (4)

To a solution of compound 3 (18 g, 0.049 mol) in methanol (30 mL) was added methanolic ammonia (7M solution, 100 mL) at 0° C. under nitrogen atmosphere. Reaction mixture was stirred at RT for 16 h. After consumption of the starting material (by TLC), volatiles were evaporated under vacuum. The crude was purified by column chromatography by eluting with 5% MeOH/CH₂Cl₂ to afford compound 4 (8 g, 54%) as a thick liquid. ¹H NMR (400 MHz, DMSO-d₆) δ 7.85 (br d, J=1.6 Hz, 2H), 5.84-5.50 (m, 2H), 3.65 (s, 3H), 3.54-3.38 (m, 2H), 3.38-3.23 (m, 2H), 2.81-2.62 (m, 1H), 2.61-2.52 (m, 1H), 2.10-1.87 (m, 2H), 1.84-1.71 (m, 2H), 1.38, 1.33 (2s, 9H).

### Synthesis of tert-butyl 6-oxo-1,7-diazaspiro[4.6]undec-9-ene-1-carboxylate (5)

To a stirring solution of compound 4 (14 g, 0.046 mol) in THF (140 mL) was added t-BuMgCl (1M solution in THF, 140.9 mL, 0.140 mol) dropwise at 0° C. and the reaction mixture was stirred at RT for 16 h. After consumption of the starting material (by TLC), the reaction was quenched with aqueous NH₄Cl (100 mL) and extracted with EtOAc (2×200 mL). The combined organic layer was washed brine (200 mL). The organic layer was dried over Na₂SO₄ and concentrated under reduced pressure. The crude was purified by column chromatography by eluting with 2% MeOH/CH₂Cl₂ to obtain compound 5 (9 g, 72%) as a light brown solid. ¹H NMR (400 MHz, DMSO-d₆) δ 7.60, 7.43 (2s, 1H), 6.11-5.88 (m, 2H), 3.76 (br d, J=15.9 Hz, 1H), 3.50-3.34 (m, 3H), 3.22-3.11 (m, 1H), 2.16-1.94 (m, 2H), 1.91-1.69 (m, 3H), 1.37 (s, 9H); LCMS (m/z): 167.0 [(M⁺+1)-Boc].

### Synthesis of 1,7-diazaspiro[4.6]undec-9-en-6-one hydrochloride (6)

To a solution of compound 5 (1 g, 0.0037 mol) in CH₂Cl₂ (5 mL) was added HCl (2M solution in diethyl ether, 5 mL) at 0° C. under nitrogen atmosphere. Reaction mixture was stirred at RT for 16 h. After consumption of the starting material (by TLC), volatiles were evaporated under reduced pressure. The crude was triturated with Et₂O and dried under vacuum to afford compound 6 (750 mg, 98%) as a hygroscopic white solid. ¹H NMR (400 MHz, DMSO-d₆) δ 9.66 (br s, 1H), 8.78 (br s, 1H), 8.45 (br d, J=6.8 Hz, 1H), 5.86-5.60 (m, 2H), 4.05-3.88 (m, 1H), 3.60-3.52 (m, 1H), 3.21 (br s, 2H), 2.62 (br s, 2H), 2.38-2.26 (m, 1H), 2.19-1.97 (m, 2H), 1.95-1.81 (m, 1H).

### Synthesis of 1-benzyl-1,7-diazaspiro[4.6]undec-9-en-6-one (GP-1 & GP-2)

To a solution of compound 6 (1 g, 4.95 mmol) in DMF (10 mL) were added K₂CO₃ (2 g, 14.85 mmol) and benzyl bromide (0.87 mL, 7.42 mmol) at RT and stirred for 16 h. After consumption of the starting material (by TLC), diluted with water (50 mL) and extracted with EtOAc (2×100 mL). The combined organic layer was washed with brine, dried over Na₂SO₄ and concentrated under reduced pressure. The crude was purified by column chromatography by eluting with 2% MeOH/CH₂Cl₂ to afford mixture of GP-1 & GP-2 (1 g, 78%) as a light yellow solid. Mixture of GP-1 & GP-2 (1 g) was separated by normal phase chiral preparative HPLC purification to obtain GP-1 (210 mg) as a white solid and GP-2 (230 mg) as a white solid.

### GP-1:

¹H NMR (400 MHz, DMSO-d₆) δ 7.40 (br t, J=4.1 Hz, 1H), 7.35-7.25 (m, 4H), 7.22-7.16 (m, 1H), 5.88-5.75 (m, 2H), 3.88 (d, J=13.9 Hz, 1H), 3.81-3.60 (m, 3H), 2.74-2.62 (m, 3H), 2.24-2.13 (m, 2H), 1.81-1.61 (m, 3H)

LCMS (ESI): m/z 257.1 [M⁺+1]

HPLC: 99.27%

Chiral HPLC: >99%

Column: CHIRALPAK IA (250*4.6 mm, 5 μm)

Mobile Phase: A: 0.1% DEA in n-hexane

Mobile Phase: B: IPA

A:B: 95:05; Flow rate: 1.0 mL/min

Retention time: 10.278

### GP-2:

¹H NMR (400 MHz, DMSO-d₆) δ 7.40 (br s, 1H), 7.35-7.25 (m, 4H), 7.23-7.14 (m, 1H), 5.89-5.72 (m, 2H), 3.88 (d, J=13.9 Hz, 1H), 3.81-3.59 (m, 3H), 2.75-2.61 (m, 3H), 2.26-2.11 (m, 2H), 1.81-1.60 (m, 3H)

LCMS (ESI): m/z 257.1 [M⁺+1]

HPLC: 99.77%

Chiral HPLC: 99.36%

Column: CHIRALPAK IA (250*4.6 mm, 5 μm)

Mobile Phase: A: 0.1% DEA in n-hexane

Mobile Phase: B: IPA

A:B: 95:05; Flow rate: 1.0 mL/min

Retention time: 12.387

### Intermediate

### Synthesis of (Z)-1,4-dibromobut-2-ene (Int-A)

To a stirring solution of PPh₃ (100 g, 0.381 mol) in acetonitrile (500 mL) was added bromine (19.6 mL, 0.381 mol) was added dropwise at 0° C. and stirred for 1 h. (Z)-but-2-ene-1,4-diol (33.5 g, 0.381 mol) to the reaction mixture at 0° C. and the reaction mixture was stirred at 50° C. for 5 h. After consumption of the starting material (by TLC), the reaction was brought to RT, diluted with water (300 mL) and extracted with Et₂O (2×500 mL). The organic layer was dried over Na₂SO₄ and concentrated under reduced pressure. The crude was purified by column chromatography by eluting with 5% EtOAc/n-hexane to obtain Int-A (23 g, 28%) as a pale brown viscous liquid. ¹H NMR (400 MHz, DMSO-d₆): δ 5.89-5.81 (m, 2H), 4.28-4.21 (m, 4H).

### Synthetic Scheme for GH-1 and GH-2

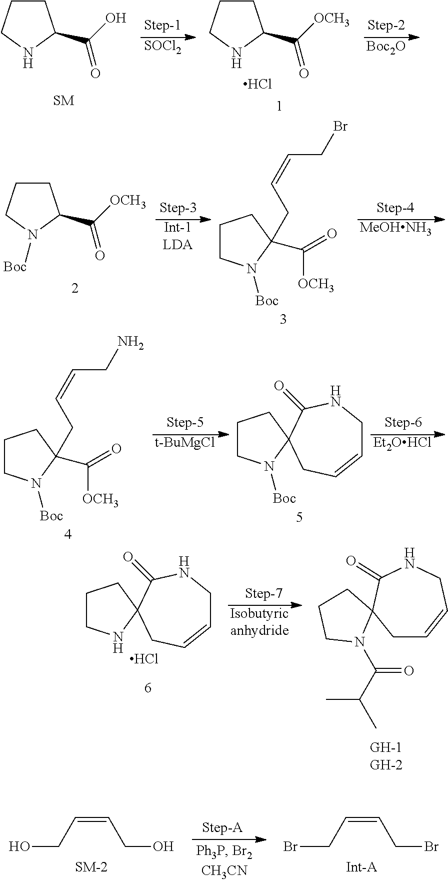

The experimental procedure for the synthesis of compound 6 and Int-A has been captured under the synthesis of GP-1 & GP-2 (as compound 6 and Int-A).

### Synthesis of 1-isobutyryl-1,7-diazaspiro[4.6]undec-9-en-6-one (GH-1 & GH-2)

To a solution of compound 6 (1.2 g, 5.94 mmol) in CH₂Cl₂ (5 mL) were added Et₃N (2.5 mL, 17.8 mmol) and isobutyric anhydride (1.4 mL, 8.91 mmol) at 0° C. and stirred at RT for 16 h.

After consumption of the starting material (by TLC), volatiles were removed under reduced pressure. The crude was purified by neutral alumina column chromatography by eluting with 5% MeOH/CH₂Cl₂ to afford mixture of GH-1 & GH-2 (1 g, 71%) as viscous liquid. Mixture of GH-1 & GH-2 (1 g) was separated by chiral preparative HPLC purification to obtain GH-1 (198 mg) as a yellow viscous liquid and GH-2 (178 mg) as a yellow viscous liquid.

### GH-1:

¹H NMR (400 MHz, DMSO-d₆) δ 7.37 (br d, J=4.6 Hz, 1H), 6.15-5.96 (m, 2H), 3.92-3.82 (m, 1H), 3.66-3.58 (m, 1H), 3.57-3.49 (m, 1H), 3.43-3.32 (m, 2H), 2.66-2.57 (m, 1H), 2.01-1.86 (m, 4H), 1.83-1.75 (m, 1H), 0.98 (d, J=6.7 Hz, 3H), 0.93 (d, J=6.8 Hz, 3H)

LCMS (ESI): m/z 237.1 [M⁺+1]

HPLC: 98.48%

Chiral HPLC: 100.00%

Column: CHIRALPAK IC (250*4.6 mm*5 μm)

Mobile Phase: A: 0.1% DEA in n-hexane

Mobile Phase: B: DCM:IPA:MeOH (80:10:10)

A:B: 50:50; Flow rate: 1.0 mL/min

Retention time: 11.517

### GH-2:

¹H NMR (400 MHz, DMSO-d₆) δ 7.38 (br d, J=4.6 Hz, 1H), 6.17-5.94 (m, 2H), 3.94-3.83 (m, 1H), 3.67-3.49 (m, 2H), 3.43-3.33 (m, 2H), 2.65-2.59 (m, 1H), 2.03-1.86 (m, 4H), 1.84-1.76 (m, 1H), 0.99 (d, J=6.8 Hz, 3H), 0.94 (d, J=6.8 Hz, 3H)

LCMS (ESI): m/z 237.1 [M⁺+1]

HPLC: 99.87%

Chiral HPLC: >99%

Column: CHIRALPAK IC (250*4.6 mm*5 μm)

Mobile Phase: A: 0.1% DEA in n-hexane

Mobile Phase: B: DCM:IPA:MeOH (80:10:10)

A:B: 50:50; Flow rate: 1.0 mL/min

Retention time: 21.881

### Synthetic Scheme for GJ-1 and GJ-2

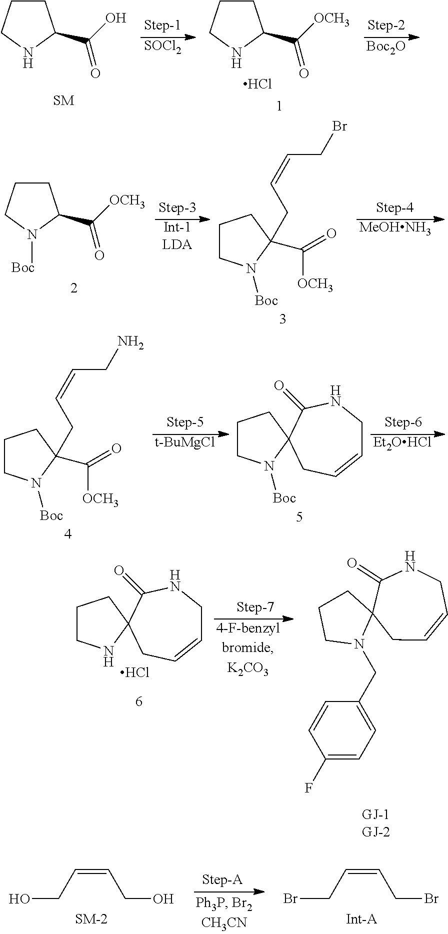

The experimental procedure for the synthesis of compound 6 and Int-A has been captured under the synthesis of GP-1 & GP-2 (as compound 6 and Int-A).

### Synthesis of 1-(4-fluorobenzyl)-1,7-diazaspiro[4.6]undec-9-en-6-one (GJ-1 & GJ-2)

To a solution of compound 6 (400 mg, 0.0019 mol) in DMF (5 mL) were added K₂CO₃ (819 mg, 0.0059 mol) and 4-fluorobenzyl bromide (0.29 mL, 0.0020 mol) at RT and stirred for 16 h. After consumption of the starting material (by TLC), diluted with water (10 mL) and extracted with EtOAc (2×10 mL). The combined organic layer was washed with brine, dried over Na₂SO₄ and concentrated under reduced pressure. The crude was washed with 20% Et₂O/n-pentane and dried to afford mixture GJ-1 & GJ-2 (420 mg, 77%) as a yellow solid. The mixture GJ-1 & GJ-2 (420 mg) was separated by chiral preparative HPLC purification to obtain GJ-1 (160 mg) as a pale yellow solid and GJ-2 (145 mg) as a pale yellow solid.

### GJ-1

¹H NMR (400 MHz, DMSO-d₆) δ 7.43-7.32 (m, 3H), 7.15-7.05 (m, 2H), 5.89-5.70 (m, 2H), 3.89 (d, J=13.8 Hz, 1H), 3.72-3.64 (m, 3H), 2.74-2.57 (m, 3H), 2.22-2.09 (m, 2H), 1.79-1.60 (m, 3H)

LCMS (ESI): m/z 275.2 [M++1]

HPLC: 97.87%

Chiral HPLC: >99%

Column: CHIRALPAK IC3 (150×4.6 mm) 3.0 □m

Mobile Phase: A: 0.1% DEA in n-hexane

Mobile Phase: B: DCM:MeOH (50:50)

A:B: 90:10; Flow rate: 1.0 mL/min

Retention time: 6.542

### GJ-2

1H NMR (400 MHz, DMSO-d₆) δ 7.44-7.32 (m, 3H), 7.16-7.04 (m, 2H), 5.87-5.72 (m, 2H), 3.89 (d, J=13.8 Hz, 1H), 3.76-3.56 (m, 3H), 2.74-2.57 (m, 3H), 2.25-2.09 (m, 2H), 1.85-1.57 (m, 3H)

LCMS (ESI): m/z 275.2 [M++1]

HPLC: 97.67%

Chiral HPLC: 100.00%

Column: CHIRALPAK IC3 (150×4.6 mm) 3.0 □m

Mobile Phase: A: 0.1% DEA in n-hexane

Mobile Phase: B: DCM:MeOH (50:50)

A:B: 90:10; Flow rate: 1.0 mL/min

Retention time: 7.198

### Synthetic Scheme for GQ-1 & GQ-2

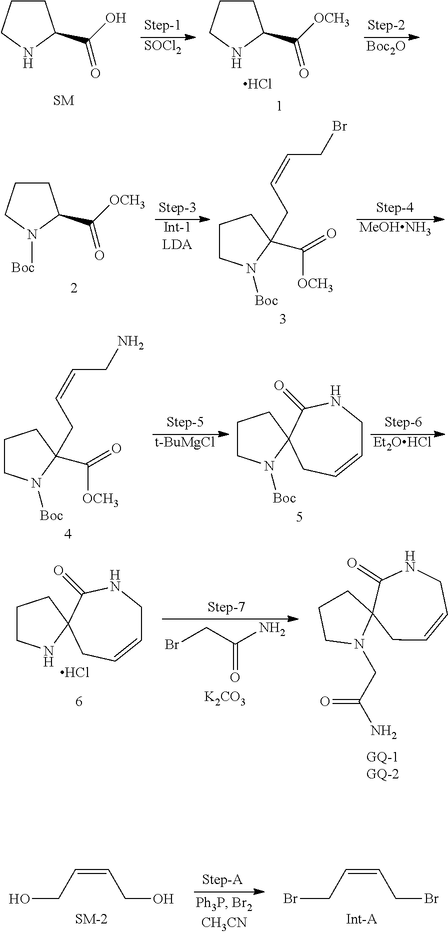

The experimental procedure for the synthesis of compound 6 and Int-A has been captured under GP-1 & GP-2 (as compound 6 and Int-A, respectively).

### Synthesis of 2-(6-oxo-1,7-diazaspiro[4.6]undec-9-en-1-yl)acetamide (GQ-1 & GQ-2)

To a solution of compound 6 (1 g, 4.95 mmol) in DMF (10 mL) were added K₂CO₃ (2 g, 14.85 mmol) and 2-bromoacetamide (681 mg, 7.40 mmol) at RT and stirred for 16 h. After consumption of the starting material (by TLC), diluted with water (50 mL) and extracted with EtOAc (2×100 mL). The combined organic layer was washed with brine, dried over Na₂SO₄ and concentrated under reduced pressure. The crude was purified by column chromatography by eluting with 5% MeOH/CH₂Cl₂ to afford mixture of GQ-1 & GQ-2 (800 mg, 72%) as a white solid. Mixture of GQ-1 & GQ-2 (800 mg) was separated by normal phase chiral preparative HPLC purification to obtain GQ-1 (190 mg) as a white solid and GQ-2 (208 mg) as a white solid.

### GQ-1

¹H NMR (400 MHz, DMSO-d₆) δ 7.41 (br d, J=6.8 Hz, 2H), 7.03 (br s, 1H), 5.81-5.60 (m, 2H), 3.81-3.68 (m, 1H), 3.58-3.50 (m, 1H), 3.27 (d, J=16.4 Hz, 1H), 3.00 (d, J=16.4 Hz, 1H), 2.85-2.75 (m, 2H), 2.52 (br s, 1H), 2.15-2.03 (m, 2H), 1.87-1.62 (m, 3H)

LCMS (ESI): m/z 224.0 [M⁺+1]

HPLC: 97.17%

Chiral HPLC: 100.00%

Column: CHIRALPAK IC (250*4.6 mm*3 μm)

Mobile Phase: A: 0.1% DEA in n-hexane

Mobile Phase: B: DCM:MeOH (50:50)

A:B: 40:60; Flow rate: 1.0 mL/min

Retention time: 9.562

### GC-2

¹H NMR (400 MHz, DMSO-d₆) δ 7.41 (br d, J=7.0 Hz, 2H), 7.03 (br s, 1H), 5.81-5.65 (m, 2H), 3.82-3.69 (m, 1H), 3.58-3.50 (m, 1H), 3.27 (d, J=16.4 Hz, 1H), 3.00 (d, J=16.4 Hz, 1H), 2.86-2.74 (m, 2H), 2.52 (br s, 1H), 2.16-2.05 (m, 2H), 1.89-1.62 (m, 3H)

LCMS (ESI): m/z 224.0 [M⁺+1]

HPLC: 98.61%

Chiral HPLC: 100.00%

Column: CHIRALPAK IC (250*4.6 mm*3 μm)

Mobile Phase: A: 0.1% DEA in n-hexane

Mobile Phase: B: DCM:MeOH (50:50)

A:B: 40:60; Flow rate: 1.0 mL/min

Retention time: 13.388

### Synthetic Scheme for GT-1 & GT-2

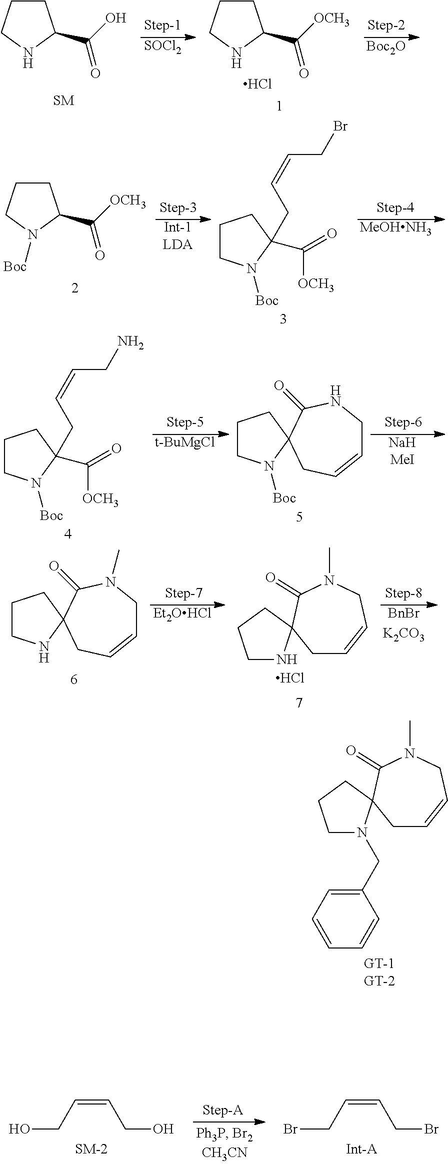

The experimental procedure for the synthesis of compound 5 and Int-A has been captured under GP-1 & GP-2 (as compound 5 and Int-A respectively).

### Synthesis of tert-butyl 7-methyl-6-oxo-1,7-diazaspiro[4.6]undec-9-ene-1-carboxylate (6)

To a stirred solution of compound 5 (2 g, 7.51 mmol) in DMF (10 mL) was added NaH (50% suspension in mineral oil, 270 mg, 11.2 mmol) at 0° C. under nitrogen atmosphere and stirred at RT for 30 minutes. The reaction mixture was cooled to 0° C., methyl iodide (0.92 mL, 15.03 mmol) was added and stirred at RT for 4 h. After consumption of the starting material (by TLC), quenched with ice water (20 mL) and extracted with EtOAc (2×50 mL). The combined organic layer was washed with brine, dried over Na₂SO₄ and concentrated under reduced pressure to afford compound 6 (1.5 g), which was taken to next step without any further purification.

### Synthesis of 7-methyl-1,7-diazaspiro[4.6]undec-9-en-6-one hydrochloride (7)

To a stirred solution of compound 6 (1.5 g, 5.375 mmol) in CH₂Cl₂ (10 mL) was added HCl (2M solution in diethyl ether, 10 mL) at 0° C. under nitrogen atmosphere and the reaction mixture was stirred at RT for 2 h. After consumption of the starting material (by TLC), volatiles were evaporated under reduced pressure. The crude was triturated with Et₂O and dried under vacuum to afford compound 7 (1 g, 86%) as a light brown solid.

### Synthesis of 1-benzyl-7-methyl-1,7-diazaspiro[4.6]undec-9-en-6-one (GT-1 & GT-2)

To a mixture of compound 7 (1 g, 4.62 mmol) in DMF (10 mL) were added K₂CO₃ (1.9 g, 13.8 mmol) and benzyl bromide (0.29 mL, 5.54 mmol) at RT and stirred for 16 h. After consumption of the starting material (by TLC), diluted with ice water (10 mL) and extracted with EtOAc (2×50 mL). The combined organic layer was washed with brine, dried over Na₂SO₄ and concentrated under reduced pressure. The crude was purified by column chromatography eluting with 5% EtOAc/n-hexane and dried to afford mixture GT-1 & GT-2 (750 mg, 62%) as a viscous liquid. Mixture of GT-1 & GT-2 (750 mg) was separated by chiral preparative HPLC purification to obtain GT-1 (210 mg) as a viscous liquid and GT-2 (250 mg) as a viscous liquid.

### GT-1

¹H NMR (400 MHz, DMSO-d₆) δ 7.35-7.26 (m, 4H), 7.23-7.16 (m, 1H), 5.87-5.75 (m, 2H), 4.25-4.16 (m, 1H), 3.95-3.84 (m, 2H), 3.62 (d, J=13.8 Hz, 1H), 2.91 (s, 3H), 2.72-2.61 (m, 3H), 2.31-2.17 (m, 2H), 1.80-1.57 (m, 3H)

LCMS (ESI): m/z 271.2 [M⁺+1]

HPLC: 99.56%

Chiral HPLC: >99%

Column: CHIRALPAK IG (150*4.6 mm*5 μm)

Mobile Phase: A: 0.1% DEA in n-hexane

Mobile Phase: B: DCM:MeOH (80:20)

A:B: 60:40; Flow rate: 1.0 mL/min

Retention time: 5.534

### GT-2

¹H NMR (400 MHz, DMSO-d₆) δ 7.37-7.25 (m, 4H), 7.23-7.16 (m, 1H), 5.89-5.75 (m, 2H), 4.26-4.15 (m, 1H), 3.95-3.84 (m, 2H), 3.62 (d, J=13.8 Hz, 1H), 2.91 (s, 3H), 2.72-2.62 (m, 3H), 2.31-2.17 (m 2H), 1.81-1.58 (m 3H)

LCMS (ESI): m/z 271.3 [M⁺+1]

HPLC: 98.66%

Chiral HPLC: >99%

Column: CHIRALPAK IG (150*4.6 mm*5 μm)

Mobile Phase: A: 0.1% DEA in n-hexane

Mobile Phase: B: DCM:MeOH (80:20)

A:B: 60:40; Flow rate: 1.0 mL/min

Retention time: 6.101

### Synthetic Scheme for GI-1 & GI-2

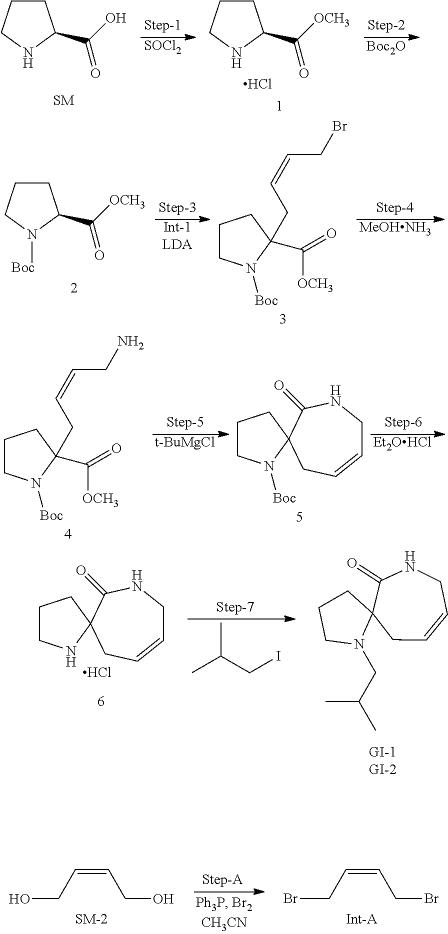

The experimental procedure for the synthesis of compound 6 and Int-A has been captured under the synthesis of GP-1 & GP-2 (as compound 6 and Int-A respectively).

### Synthesis of 1-isobutyl-1,7-diazaspiro[4.6]undec-9-en-6-one (GI-1 & GI-2)

To a solution of compound 6 (800 mg, 3.96 mmol) in DMF (5 mL) was added K₂CO₃ (1.63 g, 11.8 mmol) at 0° C. and stirred for 20 minutes. Isobutyl iodide (1.04 mL, 5.94 mmol) was added at 0° C. and continued stirring at RT for 16 h. After consumption of the starting material (by TLC), quenched with water (10 mL) and extracted with EtOAc (2×10 mL). The combined organic layer was dried over Na₂SO₄ and concentrated under reduced pressure. The crude was purified by column chromatography by eluting with 5% MeOH/CH₂Cl₂ to afford mixture of GI-1 & GI-2 (600 mg, 68%) as viscous liquid. Mixture of GI-1 & GI-2 (1.05 g, 2 batches) was separated by chiral preparative HPLC purification to obtain GI-1 (199 mg) as a colourless viscous liquid and GI-2 (184 mg) as a colourless viscous liquid.

### GI-1

¹H NMR (400 MHz, DMSO-d₆) δ 7.28 (br s, 1H), 5.92-5.73 (m, 2H), 3.79-3.64 (m, 1H), 3.61-3.49 (m 1H), 2.95-2.90 (m 1H), 2.78-2.65 (m 1H), 2.62-2.53 (m 2H), 2.24 (dd, J=9.0, 12.4 Hz, 1H), 2.13-2.06 (m, 1H), 2.02-1.96 (m, 1H), 1.80-1.50 (m, 4H), 0.84 (dd, J=0.9, 6.5 Hz, 6H)

LCMS (ESI): m/z 223.0 [M++1]

HPLC: 96.49%

Chiral HPLC: >99%

Column: CHIRALPAK IC (250*4.6 mm*3 μm)

Mobile Phase: A: 0.1% DEA in n-hexane

Mobile Phase: B: IPA

A:B: 95:05; Flow rate: 1.0 mL/min

Retention time: 15.820

### GI-2

¹H NMR (400 MHz, DMSO-d₆) δ 7.29 (br s, 1H), 5.92-5.74 (m, 2H), 3.78-3.66 (m, 1H), 3.60-3.49 (m, 1H), 2.96-2.91 (m, 1H), 2.77-2.65 (m, 1H), 2.62-2.53 (m, 2H), 2.24 (dd, J=9.0, 12.4 Hz, 1H), 2.13-2.07 (m, 1H), 2.02-1.97 (m, 1H), 1.78-1.53 (m, 4H), 0.84 (dd, J=0.9, 6.6 Hz, 6H)

LCMS (ESI): m/z 223.1 [M⁺+1]

HPLC: 95.27%

Chiral HPLC: 99.49%

Column: CHIRALPAK IC (250*4.6 mm*3 μm)

Mobile Phase: A: 0.1% DEA in n-hexane

Mobile Phase: B: IPA

A:B: 95:05; Flow rate: 1.0 mL/min

Retention time: 21.641

### Synthetic Scheme for GC-1 & GC-2

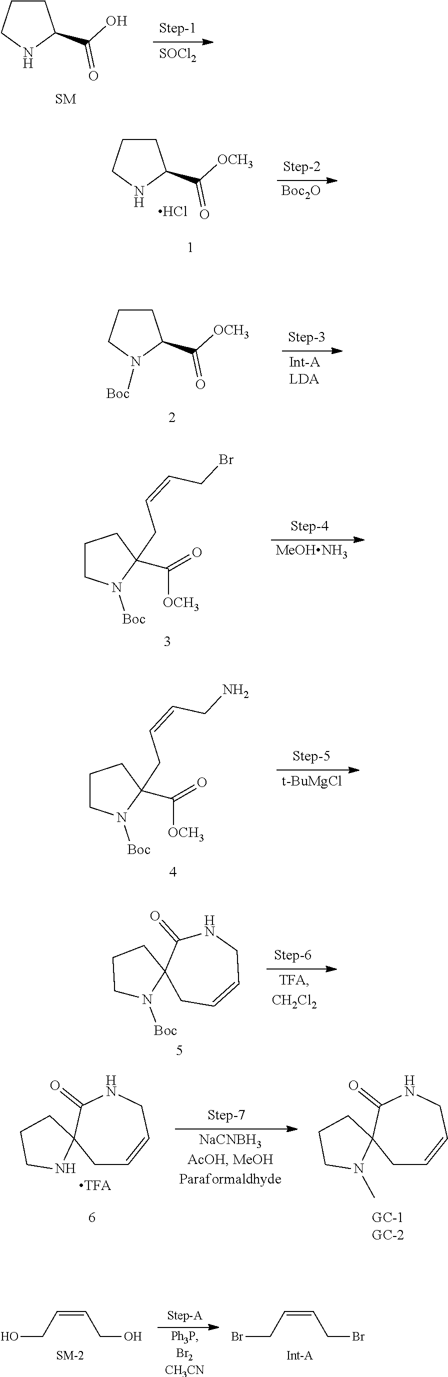

The experimental procedure for the synthesis of compound 5 and Int-A has been captured under the synthesis of GP-1 & GP-2 (as compound 5 and Int-A respectively).

### Synthesis of 1,7-diazaspiro[4.6]undec-9-en-6-one 2,2,2-trifluoroacetaldehyde (6)

To a solution of compound 5 (2 g, 7.51 mmol) in CH₂Cl₂ (20 mL) was added trifluoroacetic acid (5.95 mL) drop wise at 0° C. under nitrogen atmosphere and the reaction mixture was stirred at RT for 3 h. After consumption of the starting material (by TLC), volatiles were evaporated under reduced pressure. The crude was triturated with Et₂O and dried under vacuum to afford compound 6 (1.5 g), and was taken to next step without any further purification.

### Synthesis of 1-methyl-1,7-diazaspiro[4.6]undec-9-en-6-one (GC-1 & GC-2)

To a solution of compound 6 (1.5 g, 5.35 mmol) and paraformaldehyde (241 mg, 8.03 mmol) in methanol (20 mL) were added acetic acid (0.096 mL, 1.60 mmol) at RT and stirred for 45 minutes. Sodium triacetoxyborohydride (1.012 g, 16.0 mmol) was added portion wise and stirred the reaction mixture at 50° C. for 16 h. After consumption of the starting material (by TLC), the reaction mixture was evaporated under reduced pressure. The crude was diluted with aqueous NaHCO₃ (100 mL) and extracted with EtOAc (2×100 mL). The combined organic layer was washed with brine (50 mL), dried over Na₂SO₄ and concentrated under reduced pressure. The residue was purified by column chromatography by eluting with 5% MeOH/CH₂Cl₂ to afford mixture of GC-1 & GC-2 (750 mg, 77%). Mixture of GC-1 & GC-2 (750 mg) was separated by normal phase chiral preparative HPLC purification to obtain GC-1 (132 mg) as a thick brown viscous liquid and GC-2 (130 mg) as a thick brown viscous liquid.

### GC-1

¹H NMR (400 MHz, DMSO-d₆) δ 7.35 (br s, 1H), 5.84-5.73 (m, 2H), 3.83-3.70 (m, 1H), 3.59-3.47 (m, 1H), 2.88-2.78 (m, 1H), 2.75-2.68 (m, 1H), 2.48-2.44 (m, 1H), 2.32 (s, 3H), 2.19-2.11 (m, 1H), 2.08-2.00 (m, 1H), 1.77-1.59 (m, 3H)

LCMS (ESI): m/z 181.0 [M⁺+1]

HPLC: 99.28%

Chiral HPLC: 100.00%

Column: CHIRALPAK IC (250*4.6 mm*5 μm)

Mobile Phase: A: 0.1% DEA in n-hexane

Mobile Phase: B: DCM:MeOH (80:20)

A:B: 80:20; Flow rate: 1.0 mL/min

Retention time: 15.562

### GC-2

¹H NMR (400 MHz, DMSO-d₆) δ 7.35 (br s, 1H), 5.85-5.72 (m, 2H), 3.85-3.71 (m, 1H), 3.58-3.47 (m, 1H), 2.86-2.78 (m, 1H), 2.75-2.67 (m, 1H), 2.48-2.44 (m, 1H), 2.32 (s, 3H), 2.19-2.10 (m, 1H), 2.08-2.00 (m, 1H), 1.77-1.58 (m, 3H)

LCMS (ESI): m/z 181.0 [M⁺+1]

HPLC: 99.19%

Chiral HPLC: 98.01%

Column: CHIRALPAK IC (250*4.6 mm*5 μm)

Mobile Phase: A: 0.1% DEA in n-hexane

Mobile Phase: B: DCM:MeOH (80:20)

A:B: 80:20; Flow rate: 1.0 mL/min

Retention time: 17.034

### Synthetic Scheme for GS-1 & GS-2

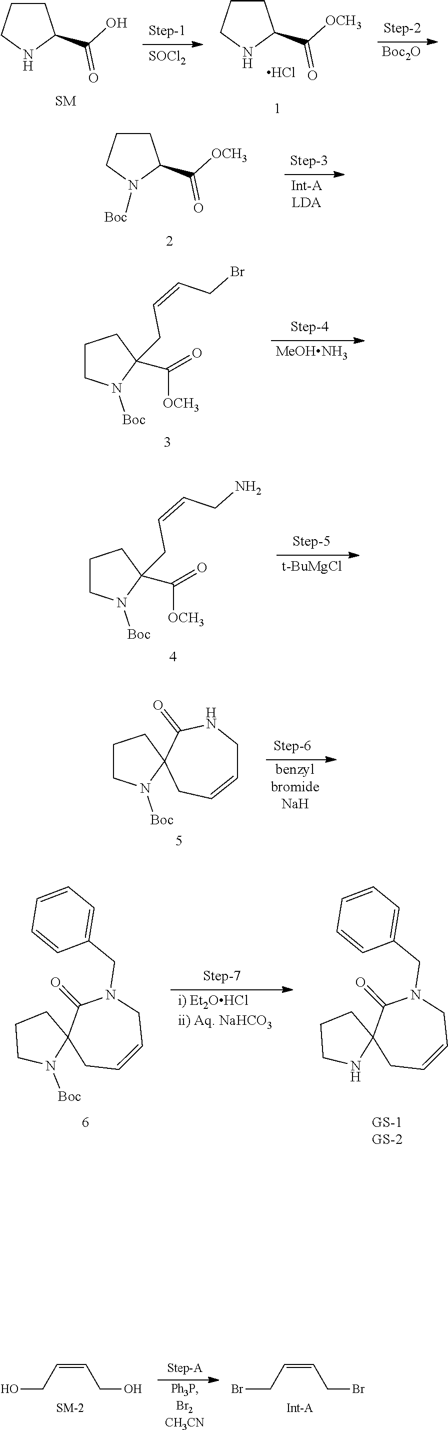

The experimental procedure for the synthesis of compound 5 and Int-A has been captured under the synthesis of GP-1 & GP-2 (as compound 5 and Int-A respectively).

### Synthesis of tert-butyl 7-benzyl-6-oxo-1,7-diazaspiro[4.6]undec-9-ene-1-carboxylate (6)

To a stirred suspension of NaH (50% suspension in mineral oil, 135 mg, 5.6 mmol) in DMF (10 mL) was added compound 5 (1 g, 3.7 mmol) at 0° C. under nitrogen atmosphere and stirred at RT for 30 minutes. The reaction mixture was cooled to 0° C., benzyl bromide (0.53 mL, 4.5 mmol) was added and stirred at RT for 2 h. After consumption of the starting material (by TLC), quenched with ice water (20 mL) and extracted with EtOAc (2×50 mL). The combined organic layer was washed with brine, dried over Na₂SO₄ and concentrated under reduced pressure. The crude was triturated with Et₂O and dried under vacuum to afford compound 6 (750 mg, 57%) as a pale yellow solid. ¹H NMR (500 MHz, DMSO-d₆) δ 7.34-7.22 (m, 5H), 6.08-5.88 (m, 2H), 4.83-4.62 (m, 1H), 4.56-4.39 (m, 2H), 4.11-3.95 (m, 1H), 3.77-3.47 (m, 2H), 3.31 (s, 2H), 2.23-2.04 (m, 2H), 1.80 (br s, 2H), 1.44, 1.29 (2s, 9H).

### Synthesis of 7-benzyl-1,7-diazaspiro[4.6]undec-9-en-6-one (GS-1 & GS-2)

To a solution of compound 6 (1.5 g, 4.2 mmol) in CH₂Cl₂ (15 mL) was added HCl (2M solution in diethyl ether, 10 mL) dropwise at 0° C. under nitrogen atmosphere and the reaction mixture was stirred at RT for 4 h. After consumption of the starting material (by TLC), volatiles were evaporated under vacuum. The crude was triturated with Et₂O and dried under reduced pressure. The crude was dissolved in EtOAc (50 mL) was added saturated aqueous NaHCO₃ (5 mL) dropwise at 0° C. and adjusted pH to 7-8. After consumption of the starting material (by TLC), organic layer was extracted and washed with brine, dried over Na₂SO₄ and concentrated under reduced pressure to afford mixture of GS-1 & GS-2 (750 mg, 62%) as a pale brown solid. Mixture of GS-1 & GS-2 (750 mg) was separated by normal phase chiral preparative HPLC purification to obtain GS-1 (210 mg) as a white solid and GS-2 (190 mg) as a pale brown viscous liquid.

### GS-1

¹H NMR (400 MHz, DMSO-d₆) δ 7.35-7.29 (m, 2H), 7.27-7.21 (m, 1H), 7.20-7.17 (m, 2H), 5.64 (d, J=1.1 Hz, 2H), 4.69-4.62 (m, 1H), 4.57-4.50 (m, 1H), 4.13-3.94 (m, 2H), 2.99-2.91 (m, 1H), 2.87-2.80 (m, 1H), 2.45-2.21 (m, 3H), 1.87-1.66 (m, 3H)

LCMS (ESI): m/z 257.2 [M⁺+1]

HPLC: 99.24%

Chiral HPLC: >99%

Column: CHIRALPAK IC (250*4.6 mm*5 μm)

Mobile Phase: A: 0.1% DEA in n-hexane

Mobile Phase: B: DCM:MeOH (80:20)

A:B: 75:25; Flow rate: 1.0 mL/min

Retention time: 7.914

### GS-2

¹H NMR (400 MHz, DMSO-d₆) δ 7.36-7.29 (m, 2H), 7.27-7.21 (m, 1H), 7.20-7.16 (m, 2H), 5.67-5.61 (m, 2H), 4.69-4.61 (m, 1H), 4.58-4.50 (m, 1H), 4.16-4.06 (m, 1H), 4.00-3.91 (m, 1H), 2.97-2.88 (m, 1H), 2.85-2.77 (m, 1H), 2.42-2.21 (m, 3H), 1.83-1.65 (m, 3H)

LCMS (ESI): m/z 257.2 [M⁺+1]

HPLC: 99.60%

Chiral HPLC: 98.92%

Column: CHIRALPAK IC (250*4.6 mm*5 μm)

Mobile Phase: A: 0.1% DEA in n-hexane

Mobile Phase: B: DCM:MeOH (80:20)

A:B: 75:25; Flow rate: 1.0 mL/min

Retention time: 9.178

### Synthetic Scheme for GK-1 & GK-2

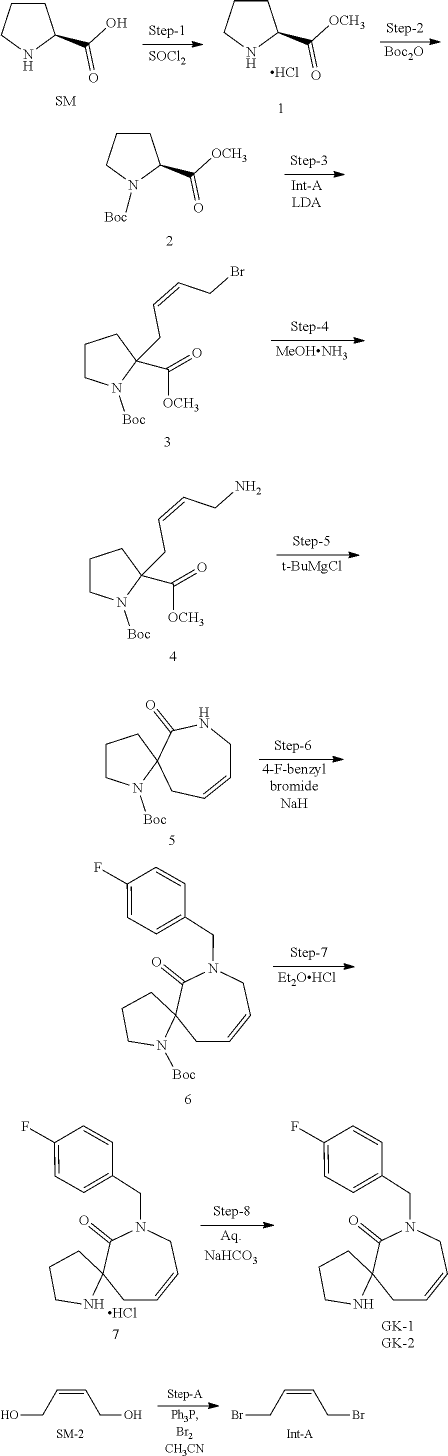

The experimental procedure for the synthesis of compound 5 and Int-A has been captured under synthesis of GP-1 & GP-2 (as compound 5 and Int-A respectively).

### Synthesis of tert-butyl 7-(4-fluorobenzyl)-6-oxo-1,7-diazaspiro[4.6]undec-9-ene-1-carboxylate (6)

To a stirred solution of compound 5 (700 mg, 2.63 mmol) in DMF (10 mL) was added NaH (50% suspension in mineral oil, 189 mg, 39.4 mmol) at 0° C. under nitrogen atmosphere and stirred at RT for 30 minutes. The reaction mixture was cooled to 0° C., 4-fluorobenzyl bromide (0.49 mL, 39.4 mmol) and stirred at RT for 2 h. After consumption of the starting material (by TLC), quenched with ice water (20 mL) and extracted with EtOAc (2×50 mL). The combined organic layer was washed with brine, dried over Na₂SO₄ and concentrated under reduced pressure. The crude was purified by column chromatography eluting with 2% MeOH/CH₂Cl₂ and dried to afford compound 6 (900 mg, 91%) as a viscous liquid. ¹H NMR (400 MHz, DMSO-d₆) δ 7.55-7.47 (m, 2H), 7.42-7.28 (m, 2H), 6.10-5.91 (m, 2H), 4.72 (s, 2H), 4.54-4.44 (m, 2H), 4.05-4.01 (m, 1H), 3.80-3.54 (m, 1H), 3.39 (br t, J=6.5 Hz, 2H), 2.10-2.01 (m 1H), 1.98-1.89 (m 1H), 1.80 (br s, 2H), 1.41, 1.30 (2s, 9H).

LCMS (m/z): 275.0 [(M⁺+1)-Boc].

### Synthesis of 7-(4-fluorobenzyl)-1,7-diazaspiro[4.6]undec-9-en-6-one hydrochloride (7)

To a solution of compound 6 (900 mg, 2.40 mmol) in CH₂Cl₂ (5 mL) was added HCl (2M solution in diethyl ether, 5 mL) dropwise at 0° C. under nitrogen atmosphere and the reaction mixture was stirred at RT for 2 h. After consumption of the starting material (by TLC), volatiles were evaporated under vacuum. The crude was triturated with Et₂O and dried under vacuum to afford compound 7 (659 mg, crude), and the crude was taken to next step without any further purification.

### Synthesis of 7-(4-fluorobenzyl)-1,7-diazaspiro[4.6]undec-9-en-6-one (GK-1 & GK-2)

To a solution of compound 7 (659 mg, 2.40 mmol) in EtOAc (50 mL) was added saturated aqueous NaHCO₃ (5 mL) dropwise at 0° C. and adjusted pH to 7-8. After consumption of the starting material (by TLC), organic layer was extracted and washed with brine, dried over Na₂SO₄ and concentrated under reduced pressure to afford mixture of GK-1 & GK-2 (600 mg, 91%). Mixture of GK-1 & GK-2 (600 mg) was separated by normal phase chiral preparative HPLC purification to obtain GK-1 (202 mg) as a pale brown viscous liquid and GK-2 (160 mg) as a pale brown viscous liquid.

### GK-1

¹H NMR (400 MHz, DMSO-d₆) δ 7.25-7.19 (m, 2H), 7.17-7.10 (m, 2H), 5.66-5.57 (m, 2H), 4.63-4.56 (m, 1H), 4.54-4.47 (m, 1H), 4.22-4.13 (m, 1H), 3.94-3.85 (m, 1H), 2.91-2.83 (m, 1H), 2.77-2.69 (m, 1H), 2.38-2.29 (m, 1H), 2.28-2.17 (m, 2H), 1.76-1.60 (m, 3H)

LCMS (ESI): m/z 275.2 [M⁺+1]

HPLC: 98.94%

Chiral HPLC: >99%

Column: CHIRALPAK IA (250×4.6 mm) 5 m

Mobile Phase: A: 0.1% DEA in n-hexane

Mobile Phase: B: EtOH

A:B: 80:20; Flow rate: 1.0 mL/min

Retention time: 7.037

### GK-2

¹H NMR (400 MHz, DMSO-d₆) δ 7.27-7.19 (m, 2H), 7.17-7.09 (m, 2H), 5.70-5.54 (m, 2H), 4.65-4.56 (m, 1H), 4.54-4.47 (m, 1H), 4.22-4.10 (m, 1H), 3.95-3.85 (m, 1H), 2.93-2.83 (m 1H), 2.78-2.68 (m 1H), 2.39-2.29 (m 1H), 2.27-2.17 (m 2H), 1.78-1.59 (m 3H)

LCMS (ESI): m/z 275.2 [M⁺+1]

HPLC: 98.57%

Chiral HPLC: >99%

Column: CHIRALPAK IA (250×4.6 mm) 5 m

Mobile Phase: A: 0.1% DEA in n-hexane

Mobile Phase: B: EtOH

A:B: 80:20; Flow rate: 1.0 mL/min

Retention time: 8.549

### Synthetic Scheme for GL-1 & GL-2

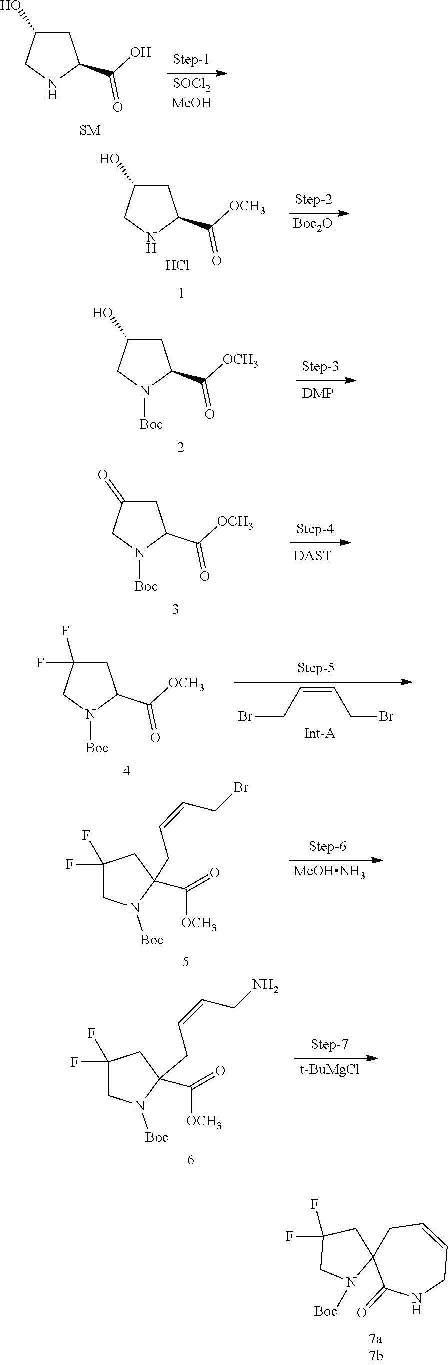

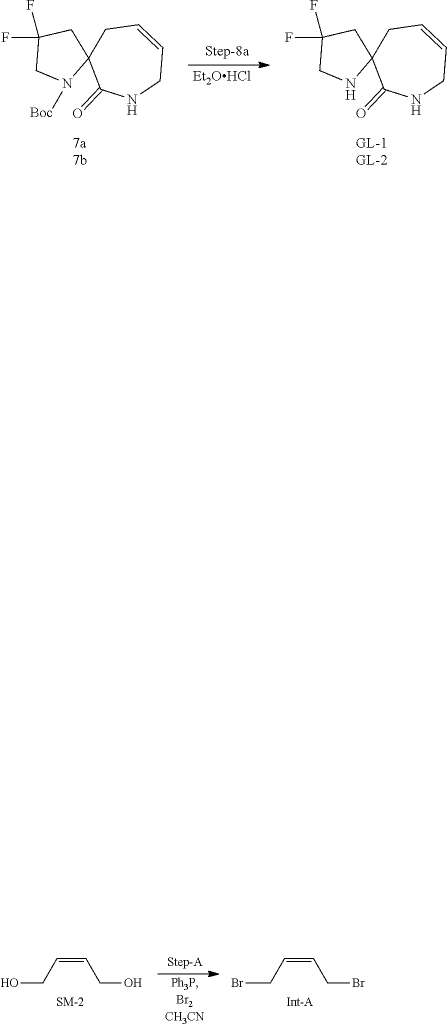

### Synthesis of methyl (2S,4R)-4-hydroxypyrrolidine-2-carboxylate hydrochloride (1)

To a stirring suspension of (2S,4R)-4-hydroxypyrrolidine-2-carboxylic acid (SM) (100 g, 0.762 mol) in methanol (1 L) was added thionyl chloride (100 mL, 1.372 mol) dropwise at 0° C. The reaction mixture was brought to room temperature and stirred for 16 h. After consumption of the starting material (by TLC), volatiles were evaporated under reduced pressure. The crude was triturated with Et₂O and dried under vacuum to afford compound 1 (130 g, 93%) as a white solid.

¹H NMR (400 MHz, DMSO-d₆) δ 5.57 (br s, 1H), 4.51-4.38 (m, 2H), 3.76 (s, 3H), 3.34 (br d, J=4.3 Hz, 2H), 3.08 (d, J=12.0 Hz, 1H), 2.25-2.16 (m, 1H), 2.14-2.04 (m, 1H).

LCMS: m/z 145.9 [M⁺+1-HCl].

### Synthesis of 1-(tert-butyl) 2-methyl (2S,4R)-4-hydroxypyrrolidine-1,2-dicarboxylate (2)

To a solution of compound 1 (130 g, 0.716 mol) in CH₂Cl₂ (1.2 L) was added Et₃N (301 mL, 2.14 mol) at 0° C. and stirred for 15 minutes. Boc₂O (197 mL, 0.859 mol) was added drop wise at 0° C. and the reaction mixture was stirred at room temperature for 16 h. After consumption of the starting material (by TLC), the reaction was diluted with ice water (500 mL) and extracted with CH₂Cl₂ (3×400 mL). Combined organic layer was washed with brine, dried over Na₂SO₄ and concentrated under reduced pressure. The crude was purified by column chromatography by eluting 30% EtOAc/n-hexane to obtain compound 2 (161 g, 91%) as a viscous liquid.

¹H NMR (400 MHz, DMSO-d₆) δ 5.08 (d, J=3.6 Hz, 1H), 4.28-4.17 (m, 2H), 3.67-3.61 (m, 3H), 3.44-3.34 (m, 1H), 3.29-3.23 (m, 1H), 2.17-2.05 (m, 1H), 1.95-1.82 (m, 1H), 1.41, 1.31 (2s, 9H).

LCMS (ESI): m/z 145.9 [(M⁺+1)-Boc].

### Synthesis of 1-(tert-butyl) 2-methyl 4-oxopyrrolidine-1,2-dicarboxylate (3)

To a stirring solution of compound 2 (5 g, 20.3 mmol) in CH₂Cl₂ (100 mL) were added Dess-Martin periodinane (25.9 g, 61.15 mmol) and NaHCO₃ (3.51 g, 40.7 mmol) at 0° C. under nitrogen atmosphere. The reaction mixture was stirred at room temperature for 16 h. After consumption of the starting material (by TLC), the reaction mixture was quenched with hypo solution and washed with aqueous NaHCO₃. Organic layer was washed with brine, dried over Na₂SO₄ and concentrated under reduced pressure. The crude was purified by column chromatography by eluting with 10% EtOAc/n-hexane to obtain compound 3 (4 g, 91%) as colorless oily viscous liquid.

¹H NMR (400 MHz, CDCl₃) δ 5.52 (t, J=7.0 Hz, 1H), 4.83-4.70 (m, 2H), 3.74 (s, 3H), 3.73-3.69 (m, 1H), 3.34-3.28 (m, 1H), 1.54 (s, 9H).

### Synthesis of 1-(tert-butyl) 2-methyl 4,4-difluoropyrrolidine-1,2-dicarboxylate (4)

To a stirring solution of compound 3 (4.5 g, 18.5 mmol) in CH₂Cl₂ (20 mL) was added DAST (5.9 g, 37.1 mmol) at 0° C. under nitrogen atmosphere. The reaction mixture was stirred at 0° C. for 16 h. After consumption of the starting material (by TLC), the reaction mixture was quenched with ice water and extracted with CH₂Cl₂ (2×100 mL). The combined organic layer was dried over Na₂SO₄ and concentrated under reduced pressure. The crude material was purified by column chromatography by eluting with 10% EtOAc/n-hexane to afford compound 4 (4 g, 81%) as colorless oily viscous liquid.

¹H NMR (400 MHz, CDCl₃) δ 4.61-4.40 (m, 1H), 3.93-3.71 (m, 5H), 2.82-2.60 (m, 1H), 2.55-2.37 (m 1H), 1.47, 1.43 (2s, 9H).

LCMS (ESI): m/z 166.1 [(M⁺+1)-Boc].

### Synthesis of 1-(tert-butyl) 2-methyl (Z)-2-(4-bromobut-2-en-1-yl)-4,4-difluoropyrrolidine-1,2-dicarboxylate (5)

To a stirring solution of compound 4 (4 g, 15.1 mmol) in THF (30 mL) was added LiHMDS (1M in THF, 18 mL, 18.0 mmol) at −78° C. under nitrogen atmosphere. A solution of Int-A (3.8 g, 18.0 mmol) in THF was added and the reaction mixture was stirred at −78° C. for 2 h. After consumption of the starting material (by TLC), reaction mixture was quenched with aqueous NH₄Cl (10 mL), stirred at room temperature for 30 minutes and extracted with EtOAc (2×100 L). The combined organic layer was dried over Na₂SO₄ and concentrated under reduced pressure. The crude material was purified by column chromatography by eluting with 20% EtOAc/n-hexane to afford compound 5 (2.5 g, 42%) as a colourless viscous liquid.

LCMS (ESI): m/z 298.1 [(M⁺+1)-Boc].

### Synthesis of 1-(tert-butyl) 2-methyl (Z)-2-(4-aminobut-2-en-1-yl)-4,4-difluoropyrrolidine-1,2-dicarboxylate (6)

To a solution of compound 5 (2.5 g, 6.29 mmol) in MeOH (2 mL) was added methanolic ammonia (7N solution, 10 mL) at room temperature in a sealed tube under nitrogen atmosphere. The reaction mixture was stirred at 50° C. for 16 h. After consumption of the starting material (by TLC), volatiles were evaporated under reduced pressure. The crude material was purified by neutral alumina column chromatography by eluting with 10% MeOH/CH₂Cl₂ to afford compound 6 (2 g, 95%) as viscous liquid.

¹H NMR (400 MHz, CDCl₃) δ 6.09-5.54 (m, 2H), 4.25-3.70 (m, 7H), 3.25-2.69 (m, 2H), 2.66-2.47 (m, 2H), 1.45, 1.44 (2s, 9H).

### Synthesis of tert-butyl 3,3-difluoro-6-oxo-1,7-diazaspiro[4.6]undec-9-ene-1-carboxylate (7)

To a stirring solution of compound 6 (2 g, 5.97 mmol) in THF (15 mL) was added t-BuMgCl (1M solution in THF, 17 mL, 17.0 mmol) at 0° C. under nitrogen atmosphere. The reaction mixture was brought to room temperature and stirred for 16 h. After consumption of the starting material (by TLC), reaction mixture was quenched with aqueous NH₄Cl (10 mL) and extracted with EtOAc (2×100 L). The combined organic layer was dried over Na₂SO₄ and concentrated under reduced pressure. The crude was purified by column chromatography by eluting 5% MeOH/CH₂Cl₂ to afford compound 7 (1.2 g, 66%) as thick yellow viscous liquid. Compound 7 (1.2 g) was separated by chiral preparative HPLC purification to obtain compound 7a (400 mg) as yellow viscous liquid and compound 7b (400 mg) as a yellow viscous liquid.

### Compound 7a

¹H NMR (400 MHz, CDCl₃) δ 6.15-6.02 (m, 2H), 5.94 (br s, 1H), 4.21-3.73 (m, 3H), 3.64-3.45 (m, 2H), 2.78-2.57 (m, 1H), 2.50-2.24 (m, 2H), 1.47 (br s, 9H)

LCMS (ESI): m/z 303.0 [M⁺+1]

HPLC: 95.76%

Chiral HPLC: >99.00%

Column: CHIRALPAK IE (250*4.6 mm*5 μm)

Mobile Phase: A: 0.1% DEA in n-hexane

Mobile Phase: B: EtOH

A:B: 70:30; Flow rate: 1.0 mL/min

Retention time: 5.959

### Compound 7b

¹H NMR (400 MHz, CDCl₃) δ 6.19-5.95 (m, 3H), 4.21-3.81 (m, 3H), 3.63-3.25 (m, 2H), 2.77-2.56 (m, 1H), 2.53-2.28 (m, 2H), 1.47 (br s, 9H)

LCMS (ESI): m/z 303.1 [M⁺+1]

HPLC: 97.80%

Chiral HPLC: >99.00%

Column: CHIRALPAK IE (250*4.6 mm*5 μm)

Mobile Phase: A: 0.1% DEA in n-hexane

Mobile Phase: B: EtOH

A:B: 70:30; Flow rate: 1.0 mL/min

Retention time: 7.510

### Synthesis of 3,3-difluoro-1,7-diazaspiro[4.6]undec-9-en-6-one (GL-1)

To a solution of compound 7a (300 mg, 0.99 mmol) in CH₂Cl₂ (10 mL) was added TFA (0.38 mL, 4.96 mmol) at 0° C. and stirred for 6 h. After consumption of the starting material (by TLC), reaction mixture was quenched with aqueous NaHCO₃ and extracted with EtOAc (2×100 mL). The combined organic layer was dried over Na₂SO₄ and concentrated under reduced pressure. The crude was purified by column chromatography by eluting 5% MeOH/CH₂Cl₂ to afford to afford GL-1 (171 mg, 85%) as an off white solid.

¹H NMR (400 MHz, DMSO-d₆) δ 7.82 (br s, 1H), 5.73-5.64 (m, 1H), 5.62-5.54 (m, 1H), 3.88-3.77 (m, 1H), 3.71-3.61 (m, 1H), 3.41 (br s, 1H), 3.21-3.01 (m, 2H), 2.93-2.82 (m, 1H), 2.43-2.28 (m, 2H), 2.21-2.08 (m, 1H)

LCMS (ESI): m/z 203.0 [M⁺+1]

HPLC: 97.34%

Chiral HPLC: 98.09%

Column: CHIRALPAK IA (250*4.6 mm*3 μm)

Mobile Phase: A: 0.1% DEA in n-hexane

Mobile Phase: B: DCM:MeOH (50:50)

A:B: 75:25; Flow rate: 1.0 mL/min

Retention time: 7.630

### Synthesis of 3,3-difluoro-1,7-diazaspiro[4.6]undec-9-en-6-one (GL-2)

To a solution of compound 7b (400 mg, 1.32 mmol) in CH₂Cl₂ (5 mL) was added TFA (0.5 mL, 6.62 mmol) at 0° C. and stirred for 6 h. After consumption of the starting material (by TLC), reaction mixture was quenched with aqueous NaHCO₃ and extracted with EtOAc (2×100 mL). The combined organic layer was dried over Na₂SO₄ and concentrated under reduced pressure. The crude material was purified by column chromatography by eluting 5% MeOH/CH₂Cl₂ to afford to afford GL-2 (174 mg, 65%) as an off white solid.

¹H NMR (400 MHz, DMSO-d₆) δ 7.83 (br s, 1H), 5.73-5.65 (m, 1H), 5.63-5.53 (m, 1H), 3.90-3.76 (m, 1H), 3.73-3.62 (m, 1H), 3.40 (br s, 1H), 3.22-3.02 (m, 2H), 2.95-2.80 (m, 1H), 2.45-2.28 (m, 2H), 2.22-2.07 (m, 1H)

LCMS (ESI): m/z 203.0 [M⁺+1]

HPLC: 98.82%

Chiral HPLC: >99.00%

Column: CHIRALPAK IA (250*4.6 mm*3 μm)

Mobile Phase: A: 0.1% DEA in n-hexane

Mobile Phase: B: DCM:MeOH (50:50)

A:B: 75:25; Flow rate: 1.0 mL/min

Retention time: 6.987

### Synthetic Scheme for GG-1 & GG-2

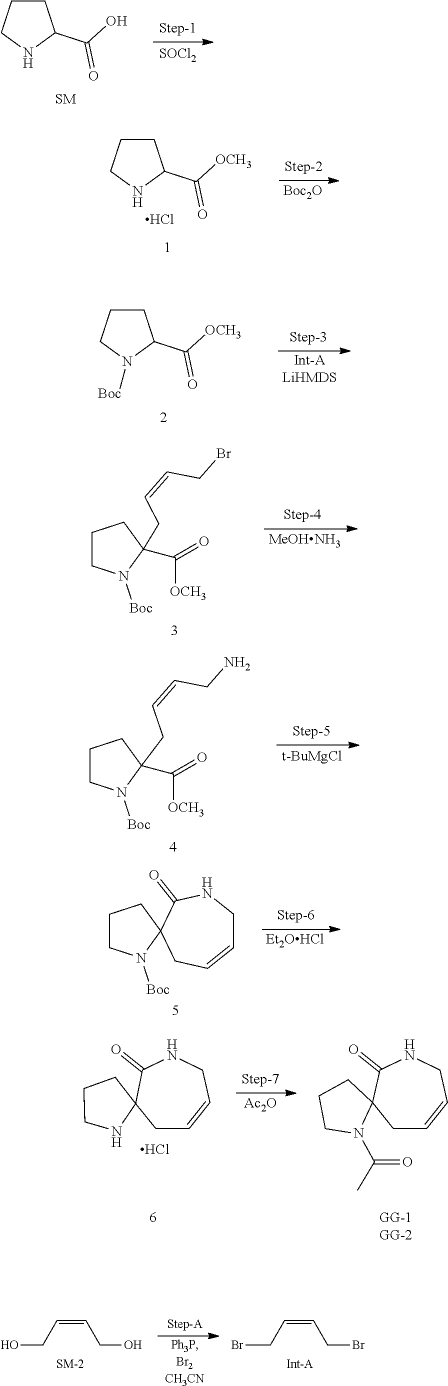

The experimental procedure for the synthesis of compound 6 and Int-A has been captured under the synthesis of GP-1 & GP-2 (as compound 6 and Int-A respectively).

### Synthesis of 1-acetyl-1,7-diazaspiro[4.6]undec-9-en-6-one (GG-1 &GG-2)

To a stirring solution of compound 6 (1.5 g, 3.65 mmol) in CH₂Cl₂ (10 mL), Et₃N (0.56 mL, 5.63 mmol) and acetic anhydride (0.35 mL, 3.75 mmol) were added at 0° C. The reaction mixture was stirred at room temperature for 16 h. After consumption of the starting material (by TLC), the reaction mixture was concentrated under reduced pressure. The residue was purified by column chromatography by eluting with 5% MeOH/CH₂Cl₂ to afford mixture of GG-1 & GG-2 (700 mg, 2 batches) as yellow semi solid. Mixture of GG-1 & GG-2 (700 mg) was purified by chiral preparative HPLC purification to obtain GG-1 (135 mg) as colorless viscous liquid and GG-2 (160 mg) as colorless viscous liquid.

### GG-1

¹H NMR (400 MHz, DMSO-d₆) δ 7.42 (br d, J=2.4 Hz, 1H), 6.09-5.94 (m, 2H), 3.85-3.78 (m, 1H), 3.57-3.34 (m, 4H), 2.03-1.94 (m, 2H), 1.93-1.79 (m, 6H)

LCMS (ESI): m/z 209.0 [M⁺+1]

HPLC: 98.50%

Chiral HPLC: >99.00%

Column: CHIRALART SA (250*4.6 mm*5 μm)

Mobile Phase: A: n-Hexane

Mobile Phase: B: EtOH:MeOH (50:50)

A:B: 45:55; Flow rate: 0.7 mL/min

Retention time: 4.416

### GG-2

¹H NMR (400 MHz, DMSO-d₆) δ 7.42 (br d, J=2.1 Hz, 1H), 6.10-5.94 (m, 2H), 3.87-3.77 (m, 1H), 3.57-3.34 (m, 4H), 2.02-1.94 (m, 2H), 1.92-1.79 (m, 6H)

LCMS (ESI): m/z 209.0 [M⁺+1]

HPLC: 96.26%

Chiral HPLC: 98.83%

Column: CHIRALART SA (250*4.6 mm*5 μm)

Mobile Phase: A: n-Hexane

Mobile Phase: B: EtOH:MeOH (50:50)

A:B: 45:55; Flow rate: 0.7 mL/min

Retention time: 4.873

### Synthetic Scheme for GD-1 & GD-2

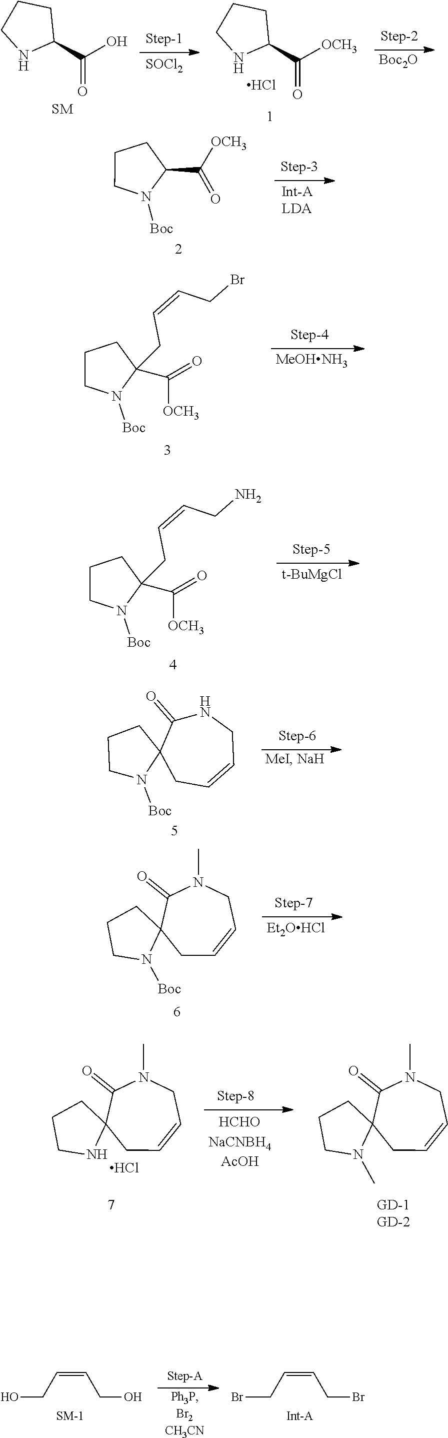

The experimental procedure for the synthesis of compound 5 has been captured under the synthesis of GP-1 & GP-2 (as compound 5).

### Synthesis of tert-butyl 7-methyl-6-oxo-1,7-diazaspiro[4.6]undec-9-ene-1-carboxylate (6)

To a stirred solution of compound 5 (2 g, 7.51 mmol) in DMF (10 mL) was added NaH (50% suspension in mineral oil, 270 mg, 11.2 mmol) at 0° C. under nitrogen atmosphere and stirred at RT for 30 minutes. The reaction mixture was cooled to 0° C., methyl iodide (0.92 mL, 15.03 mmol) was added and stirred at RT for 4 h. After consumption of the starting material (by TLC), quenched with ice water (20 mL) and extracted with EtOAc (2×50 mL). The combined organic layer was washed with brine, dried over Na₂SO₄ and concentrated under reduced pressure to afford compound 6 (1.5 g), which was taken to next step without any further purification.

### Synthesis of 7-methyl-1,7-diazaspiro[4.6]undec-9-en-6-one hydrochloride (7)

To a stirred solution of compound 6 (1.5 g, 5.375 mmol) in CH₂Cl₂ (10 mL) was added HCl (2M solution in diethyl ether, 10 mL) at 0° C. under nitrogen atmosphere and the reaction mixture was stirred at RT for 2 h. After consumption of the starting material (by TLC), volatiles were evaporated under reduced pressure. The crude was triturated with Et₂O and dried under vacuum to afford compound 7 (1 g, 86%) as a light brown solid.

### Synthesis of 1-benzyl-7-methyl-1,7-diazaspiro[4.6]undec-9-en-6-one (GD-1 & GD-2)

To a solution of compound 7 (1.9 g, 8.83 mmol) in MeOH (50 mL) were added paraformaldehyde (795 mg, 26.5 mmol), AcOH (0.15 mL, 2.65 mmol) and NaCNBH₃ (1.66 g, 26.5 mmol) at 0° C. under nitrogen atmosphere. The reaction mixture was stirred at 60° C. for 16 h. After consumption of the starting material (by TLC), cooled to room temperature and volatiles were evaporated. The reaction mixture was diluted with water (30 mL) and extracted with 10% MeOH/CH₂Cl₂ (3×50 mL). The organic layer was dried over Na₂SO₄ and concentrated under reduced pressure. The residue was purified by column chromatography by eluting 5% MeOH/CH₂Cl₂ to afford mixture of GD-1 & GD-2 (1 g) as thick liquid. Mixture of GD-1 & GD-2 (1 g) was separated by reverse phase HPLC purification followed by chiral preparative HPLC purification to obtain GD-1 (65 mg) as a thick liquid and GD-2 (60 mg) as a thick liquid.

### GD-1

¹H NMR (400 MHz, DMSO-d₆) δ 5.87-5.65 (m, 2H), 4.43-4.28 (m, 1H), 3.79-3.65 (m, 1H), 2.86 (s, 3H), 2.81-2.67 (m, 2H), 2.47-2.41 (m, 1H), 2.32-2.21 (m, 4H), 2.16-2.04 (m, 1H), 1.78-1.48 (m, 3H)

LCMS (ESI): m/z 195.0 [M⁺+1]

HPLC: 99.40%

Chiral HPLC: >99.00%

Column: CHIRALPAK IG (250×4.6×5.0 μm)

Mobile Phase: A: 0.1% DEA in n-hexane

Mobile Phase: B: IPA

A:B: 95:05; Flow rate: 1.0 mL/min

Retention time: 14.940 min

### GD-2

¹H NMR (400 MHz, DMSO-d₆) δ 5.86-5.67 (m, 2H), 4.40-4.26 (m, 1H), 3.77-3.66 (m, 1H), 2.86 (s, 3H), 2.81-2.65 (m, 2H), 2.48-2.42 (m, 1H), 2.31-2.24 (m, 4H), 2.12-2.06 (m, 1H), 1.79-1.54 (m, 3H)

LCMS (ESI): m/z 195.0 [M⁺+1]

HPLC: 99.79%

Chiral HPLC: >99.00%

Column: CHIRALPAK IG (250×4.6×5.0 μm)

Mobile Phase: A: 0.1% DEA in n-hexane

Mobile Phase: B: IPA

A:B: 95:05; Flow rate: 1.0 mL/min

Retention time: 20.082 min

### Synthetic Scheme for GU-1 & GU-2

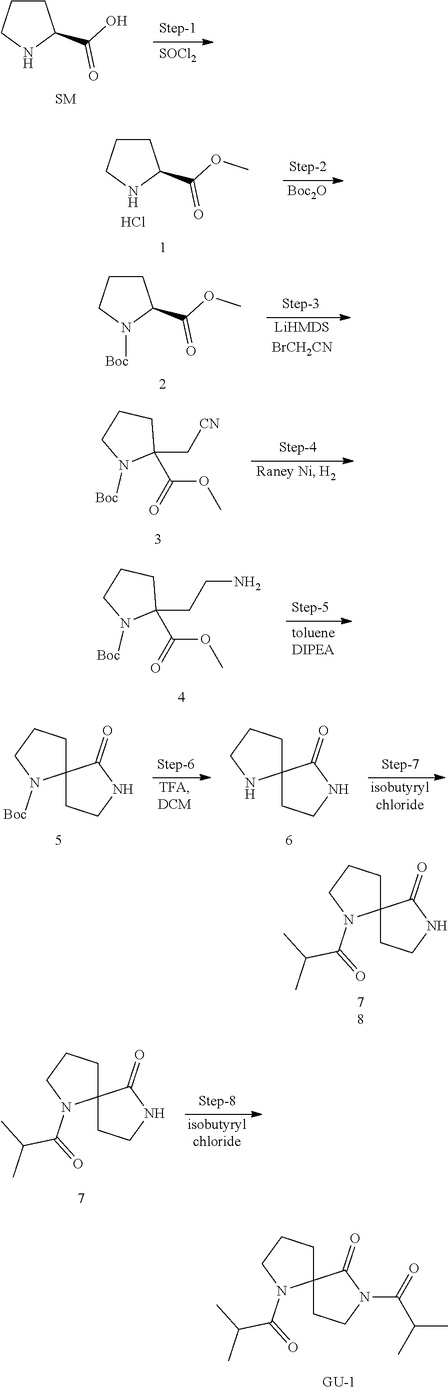

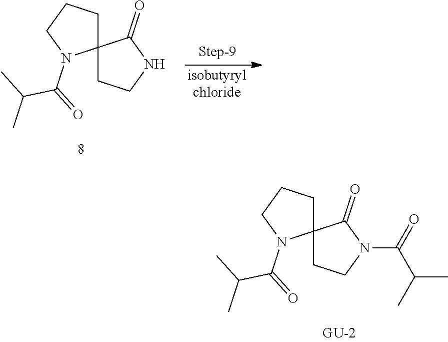 [SMILES: CC(C)C(=O)N1CCCC12CCNC2=O ; "isobutyrylchloride" ; CC(C)C(=O)N1CCC2(CCCN2C(=O)C(C)C)C1=O]

The experimental for compound 2 is captured under GG-1 and GG-2 as compound 2.

### Synthesis of 1-(tert-butyl) 2-methyl 2-(cyanomethyl)pyrrolidine-1,2-dicarboxylate (3)

To a stirred solution of 2 (20.0 g, 93.4 mmol) in THF (150 mL), LiHMDS (140 mL, 140 mmol) was added at −78° C. and stirred for 30 min. Bromoacetonitrile (12.3 mL, 102 mmol) was added at −78° C. and then stirred at room temperature for 4 h. After consumption of the starting material (by TLC), the reaction mixture was quenched with saturated NH₄Cl solution (300 mL) and extracted with EtOAc (3×300 mL). The combined organic layer was washed with brine (100 mL), dried over Na₂SO₄ and concentrated under reduced pressure to give crude product. The crude was purified by column chromatographyon SiO₂ using 40% EtOAc/hexane to afford compound 3 (15 g, 63%) as thick oil.

¹H NMR (400 MHz, DMSO-d₆) δ 3.64 (m, 3H), 3.57-3.46 (m, 1H), 3.42-3.36 (m, 1H), 3.26-3.09 (m, 5H), 2.25-2.16 (m, 2H), 2.04-1.83 (m, 2H), 1.37 (m, 9H).

### Synthesis of 1-(tert-butyl) 2-methyl 2-(2-aminoethyl)pyrrolidine-1,2-dicarboxylate (4)

To a stirred solution of compound 3 (5.0 g, 18.6 mmol) in THF and MeOH (1:1, 200 mL), Raney nickel (4.0 g) was added at room temperature and stirred for 48 h at 50° C. under H₂ atmosphere. After consumption of the starting material (by TLC), the reaction mixture was filtered through a pad of celite and the pad was washed with MeOH (50 mL). The crude was purified by column chromatography on SiO₂ using 5% MeOH/DCM to afford compound 4 (2.5 g, 50%) as thick oil.

### Synthesis of tert-butyl 6-oxo-1,7-diazaspiro[4.4]nonane-1-carboxylate (5)

To a stirred solution of compound 4 (10.0 g, 36.9 mmol) in toluene (100 mL), DIPEA (7.7 mL, 44.2 mmol) was added and reaction mixture was heated to reflux for 36 h. After consumption of the starting material (by TLC), reaction mixture was evaporated under reduced pressure. The crude was purified by column chromatography on SiO₂ to afford compound 5 as a thick oil.

¹H NMR (400 MHz, DMSO-d₆) δ 7.70 (m, 1H), 3.40-3.30 (m, 1H), 3.25-3.09 (m, 3H), 2.35-2.30 (m, 1H), 1.98-1.72 (m, 5H), 1.42 (s, 9H).

LCMS (ESI): m/z 263 [M⁺+Na].

### Synthesis of 1,7-diazaspiro[4.4]nonan-6-one (6)

To a stirred solution of 5 (1.5 g, 6.07 mmol) in DCM (7 mL), trifluoroacetic acid (7 mL) was added and stirred at room temperature for 3 h. After consumption of the starting material (by TLC), the reaction mixture was concentrated under reduced pressure to obtain compound as a TFA salt. Obtained salt was dissolved in THF (5 ml), triethylamine (5 mL) was added and then stirred at room temperature for 5 h. The crude was purified by column chromatography on SiO₂ to afford compound 6 as thick oil.

¹H NMR (400 MHz, DMSO-d₆) δ 7.67 (s, 1H), 3.20-3.03 (m, 2H), 3.01-2.97 (m, 1H), 2.78-2.65 (m, 1H), 2.20-2.14 (brs, 1H), 1.99-1.80 (m, 2H), 1.78-1.60 (m, 4H).

LCMS (ESI): m/z 141.2 [M⁺+1].

### Synthesis of 1-isobutyryl-1,7-diazaspiro[4.4]nonan-6-one (7 & 8)

To a stirred solution of 6 (0.5 g, 3.57 mmol) in DCM (10 mL), DIPEA (0.61 mL) was added followed by the addition of isobutyl chloride (0.3 mL, 2.85 mmol) at −78° C. and stirred at same temperature for 10 min. After consumption of the starting material (by TLC), the reaction mixture was concentrated under reduced pressure. The residue was purified by preparative HPLC followed by chiral HPLC to afford 7 (100 mg) & 8 (100 mg) as a white solid.

7:

¹H NMR (400 MHz, DMSO-d₆) δ 7.67 (s, 1H), 3.20-3.03 (m, 2H), 3.01-2.97 (m, 1H), 2.78-2.65 (m, 1H), 2.20-2.14 (brs, 1H), 1.99-1.80 (m, 2H), 1.78-1.60 (m, 4H), 0.96 (t, J=6.6 Hz, 6H).

LCMS (ESI): m/z 211 [M⁺+1]

HPLC: 99.56%

8:

¹H NMR (400 MHz, DMSO-d₆) δ 7.49 (s, 1H), 3.65-3.60 (m, 1H), 3.45 (q, J=9.0, 7.8 Hz, 1H), 3.33-3.16 (m, 1H), 3.11 (q, J=8.5 Hz, 1H), 2.01-1.78 (m, 7H), 0.96 (t, J=6.6 Hz, 6H).

LCMS (ESI): m/z 211 [M⁺+1]

HPLC: 99.78%

### Synthesis of 1,1′-(6-oxo-1,7-diazaspiro[4.4]nonane-1,7-diyl)bis(2-methylpropan-1-one) (GU-1)

To a stirred solution of 7 (500 g, 2.38 mmol) in CH₂Cl₂ (20 mL) were added DIPEA (0.41 mL, 2.38 mmol) followed by addition of isobutyryl chloride (0.38 mL, 3.57 mol) at 0° C. and stirred at room temperature for 16 h. After consumption of the starting material (by TLC), the reaction mixture was concentrated under reduced pressure. The residue was purified by flash column chromatography on SiO₂ by eluting with 2% MeOH/CH₂Cl₂ to obtain mixture of GU-1 (270 mg, 40%) as an off white solid.

¹H NMR (400 MHz, DMSO-d₆) δ 3.82-3.66 (m, 2H), 3.61 (quin, J=6.8 Hz, 1H), 3.56-3.40 (m, 2H), 2.67 (spt, J=6.7 Hz, 1H), 2.32-2.24 (m, 1H), 2.11-1.81 (m, 5H), 1.05 (dd, J=6.8, 15.2 Hz, 6H), 0.98 (dd, J=2.6, 6.7 Hz, 6H)

LCMS (ESI): m/z 281.2 [M⁺+1]

HPLC: 99.68%

Chiral HPLC: >99.00%

Column: CHIRALPAK IC (250*4.6 mm, 5 μm)

Mobile Phase: A: 0.1% DEA in n-hexane

Mobile Phase: B: DCM:MeOH (80:20)

A:B: 65:35; Flow rate: 1.0 mL/min

Retention time: 5.131 min

### Synthesis of 1,1′-(6-oxo-1,7-diazaspiro[4.4]nonane-1,7-diyl)bis(2-methylpropan-1-one) (GU-2)

To a stirred solution of 8 (500 g, 2.38 mmol) in CH₂Cl₂ (20 mL) were added DIPEA (0.41 mL, 2.38 mmol) followed by addition of isobutyl chloride (0.38 mL, 3.57 mol) at 0° C. and stirred at room temperature for 16 h. After consumption of the starting material (by TLC), the reaction mixture was concentrated under reduced pressure. The residue was purified by flash column chromatography by eluting with 2% MeOH/CH₂Cl₂ to obtain mixture of GU-2 (230 mg, 35%) as an off white solid.

¹H NMR (400 MHz, DMSO-d₆) δ 3.81-3.66 (m, 2H), 3.61 (quin, J=6.8 Hz, 1H), 3.56-3.40 (m, 2H), 2.67 (spt, J=6.7 Hz, 1H), 2.32-2.24 (m, 1H), 2.10-1.80 (m, 5H), 1.05 (dd, J=6.8, 15.3 Hz, 6H), 0.98 (dd, J=2.6, 6.8 Hz, 6H)

LCMS (ESI): m/z 281.1 [M⁺+1]

HPLC: 99.85%

Chiral HPLC: >99.00%

Column: CHIRALPAK IC (250*4.6 mm, 5 μm)

Mobile Phase: A: 0.1% DEA in n-hexane

Mobile Phase: B: DCM:MeOH (80:20)

A:B: 65:35; Flow rate: 1.0 mL/min

Retention time: 4.945 min

### Synthetic Scheme for CD & CE

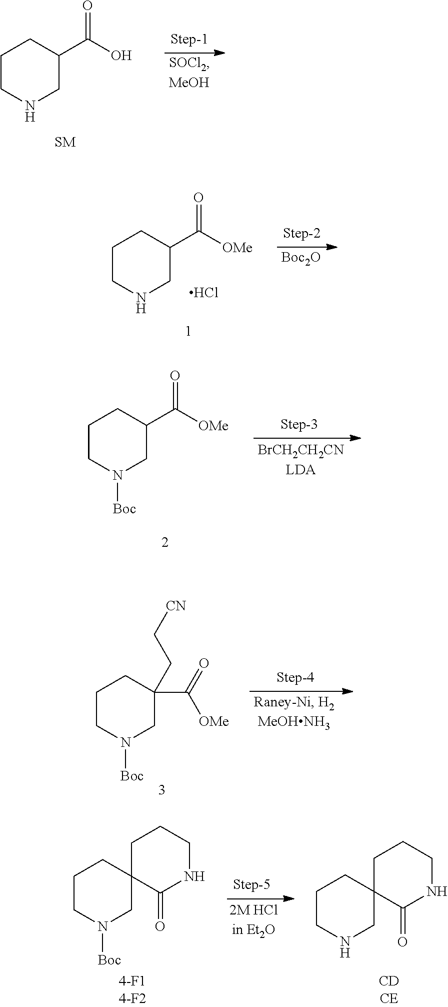

### Synthesis of methyl piperidine-3-carboxylate hydrochloride (1)

To a stirring solution of piperidine-3-carboxylic acid (SM) (50 g, 0.38 mol) in methanol (500 mL) was added thionyl chloride (50 mL, 0.696 mol) dropwise at 0° C. under nitrogen atmosphere. The reaction mixture was stirred at 80° C. for 16 h. After consumption of the starting material (by TLC), reaction mixture was brought to room temperature and volatiles were concentrated under reduced pressure. The crude syrup was triturated with Et₂O and dried under vacuum to afford compound 1 (60 g, 86%) as an off white solid. This product was taken to next step without any further purification.

### Synthesis of 1-(tert-butyl) 3-methyl piperidine-1,3-dicarboxylate (2)

To a stirring solution of compound 1 (60 g, 0.33 mol) in CH₂Cl₂ (600 mL) was added Et₃N (145 mL, 1.01 mol) at 0° C. and stirred for 10 min. Boc₂O (92 mL, 0.41 mol) was added at 0° C. and the reaction mixture was stirred at room temperature for 16 h. After consumption of the starting material (by TLC), the reaction was quenched with water (1 L) and extracted with EtOAc (2×1 L). The combined organic layer was dried over Na₂SO₄ and concentrated under reduced pressure. The crude was purified by column chromatography on SiO₂ by eluting with 10% EtOAc/hexane to obtain compound 2 (65 g, 81%) as a white solid.

¹H NMR (400 MHz, DMSO-d₆) δ 4.06-3.80 (m, 1H), 3.69-3.57 (m, 4H), 3.21-2.82 (m, 2H), 2.47-2.37 (m, 1H), 1.96-1.82 (m, 1H), 1.69-1.52 (m, 2H), 1.42-1.28 (m, 10H).

LCMS (ESI): m/z 244.0 [M+H]⁺.

### Synthesis of 1-(tert-butyl) 3-methyl 3-(2-cyanoethyl)piperidine-1,3-dicarboxylate (3)

To a stirring solution of compound 2 (10 g, 0.041 mol) in THF (100 mL) was added LDA (2M in THF, 32 mL, 0.062 mol) drop wise at −78° C. The reaction mixture was stirred at −40° C. for 1 h. Again cooled to −78° C. and 3-bromopropanenitrile (4.3 mL, 0.053 mol) was added drop wise. The reaction mixture was stirred at room temperature for 16 h. After consumption of the starting material (by TLC), the reaction was quenched with saturated aqueous NH₄Cl (1 L) and extracted with EtOAc (2×1 L). The combined organic layer was washed with brine, dried over Na₂SO₄ and concentrated under reduced pressure. The crude material was purified by column chromatography on SiO₂ by eluting 20% EtOAc/hexane to afford compound 3 (5.5 g, 45%) as a white solid.

¹H NMR (400 MHz, DMSO-d₆) δ 3.80-3.68 (m, 1H), 3.63 (s, 3H), 3.35 (br d, J=3.6 Hz, 1H), 3.18 (br d, J=13.3 Hz, 2H), 2.47-2.39 (m, 2H), 1.99-1.90 (m, 1H), 1.89-1.70 (m, 2H), 1.58-1.45 (m, 3H), 1.39 (s, 9H).

LCMS (ESI): m/z 297.1 [M+H]⁺.

### Synthesis of tert-butyl 7-oxo-2,8-diazaspiro[5.5]undecane-2-carboxylate (4)

To a stirring solution of compound 3 (5 g, 0.016 mol) in methanol (50 mL) was added Raney Nickel (5 g) and methanolic ammonia (10 mL) at room temperature. The reaction mixture was stirred under H₂ atmosphere (balloon pressure) for 16 h. After consumption of the starting material (by TLC), the reaction mixture was filtered through a pad of celite and the pad was washed with methanol (100 mL). Obtained filtrate was concentrated under reduced pressure. The crude material was purified by column chromatography on SiO₂ by eluting 5% MeOH/CH₂Cl₂ to afford mixture of compound 4 (4 g, 88%) as a white solid. Mixture of compound 4 (3.2 g) was separated by chiral preparative HPLC purification to obtain compound 4-F1 (1.2 g) as an off-white solid and compound 4-F2 (1.2 g) as an off-white solid.

### Compound 4-F1

¹H NMR (400 MHz, DMSO-d₆) δ 7.43 (br s, 1H), 4.02-3.72 (m, 2H), 3.10 (br d, J=4.6 Hz, 2H), 3.02-2.83 (m, 1H), 2.71-2.56 (m, 1H), 1.96 (br s, 1H), 1.69 (br s, 3H), 1.56-1.33 (m, 13H).

LCMS (ESI): m/z 269.1 [M+H]⁺.

### Compound 4-F2

¹H NMR (400 MHz, DMSO-d₆) δ 7.43 (br s, 1H), 4.01-3.73 (m, 2H), 3.10 (br d, J=4.6 Hz, 2H), 3.03-2.84 (m, 1H), 2.70-2.56 (m, 1H), 1.96 (br s, 1H), 1.69 (br s, 3H), 1.56-1.35 (m, 13H).

LCMS (ESI): m/z 269.1 [M+H]⁺.

### Synthesis of 2,8-diazaspiro[5.5]undecan-1-one (CD)

To a stirring solution of compound 4-F1 (1.2 g, 0.004 mol) in CH₂Cl₂ (15 mL) was added HCl (2M solution in diethyl ether, 20 mL) at 0° C. under nitrogen atmosphere. The reaction mixture was stirred at room temperature for 48 h. After consumption of the starting material (by TLC), volatiles were evaporated under reduced pressure. The crude was triturated with Et₂O and dried under reduced pressure. Obtained product was dissolved in MeOH: THF (20 mL, 1:1) and added NaHCO₃ (300 mg) portion wise at 0° C. to adjust pH to 9-10. Reaction mixture was filtered and filtrate was concentrated under reduced pressure. The crude was purified by basic alumina column chromatography by eluting 10% MeOH/CH₂Cl₂ to afford CD (300 mg) as an off-white solid.

### CD:

¹H NMR (500 MHz, DMSO-d₆) δ 7.22 (br s, 1H), 3.31 (br s, 1H), 3.12-3.01 (m, 2H), 2.73 (br d, J=12.8 Hz, 2H), 2.55 (br d, J=12.8 Hz, 1H), 2.48-2.41 (m, 1H), 1.98-1.88 (m, 2H), 1.69-1.58 (m, 2H), 1.56-1.48 (m, 1H), 1.44-1.34 (m, 3H)

LCMS (ESI): m/z 169.0 [M+H]⁺

HPLC: 99.87%

Chiral HPLC: >99.00%

Column: CHIRALPAK IG (250*4.6 mm*5 μm)

Mobile Phase: A: 0.1% DEA in n-Hexane

Mobile Phase: B: DCM:MeOH (50:50)

A:B: 70:30; Flow rate: 1.0 mL/min

Retention time: 15.663 min

### Synthesis of 2,8-diazaspiro[5.5]undecan-1-one (CE)

To a stirring solution of compound 4-F2 (1.2 g, 0.004 mol) in CH₂Cl₂ (10 mL) was added HCl (2M solution in diethyl ether, 20 mL) at 0° C. under nitrogen atmosphere. The reaction mixture was stirred at room temperature for 48 h. After consumption of the starting material (by TLC), volatiles were evaporated under reduced pressure. The crude was triturated with Et₂O and dried under reduced pressure. Obtained product was dissolved in MeOH: THF (20 mL, 1:1) and added NaHCO₃ (300 mg) portion wise at 0° C. to adjust pH to 9-10. Reaction mixture was filtered and filtrate was concentrated under reduced pressure. The crude was purified by basic alumina column chromatography by eluting 10% MeOH/CH₂Cl₂ to afford CE (300 mg) as an off-white solid.

### CE

¹H NMR (400 MHz, DMSO-d₆) δ 7.25 (br s, 1H), 3.32 (br s, 1H), 3.12-3.01 (m, 2H), 2.74 (br d, J=12.5 Hz, 2H), 2.55 (br d, J=12.5 Hz, 1H), 2.48-2.40 (m, 1H), 2.00-1.86 (m, 2H), 1.68-1.57 (m, 2H), 1.56-1.46 (m, 1H), 1.44-1.31 (m, 3H)

LCMS (ESI): m/z 169.0 [M+H]⁺

HPLC: 97.51%

Chiral HPLC: 99.77%

Column: CHIRALPAK IG (250*4.6 mm*5 μm)

Mobile Phase: A: 0.1% DEA in n-Hexane

Mobile Phase: B: DCM:MeOH (50:50)

A:B: 70:30; Flow rate: 1.0 mL/min

Retention time: 17.258 min

### Synthetic Scheme for CF & CG

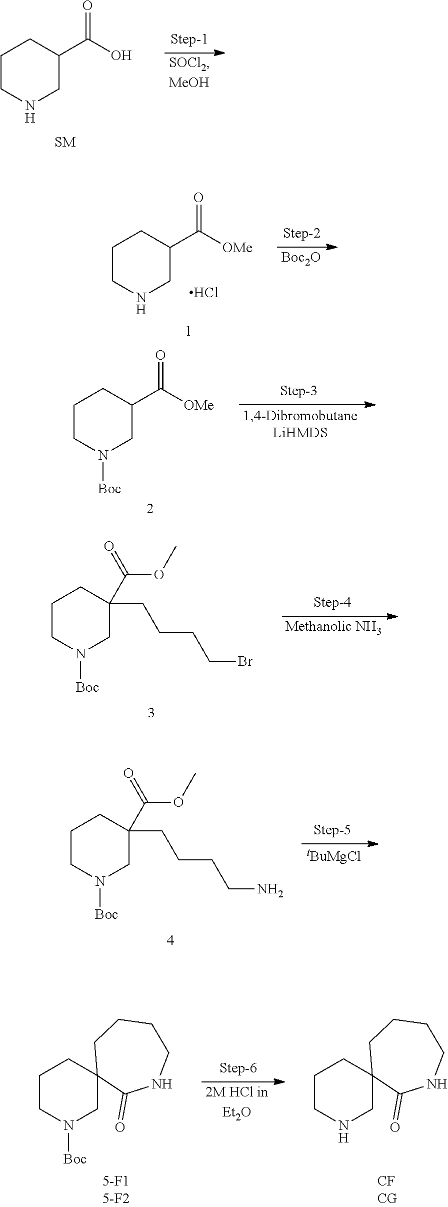

The experimental procedure for the synthesis of compound 2 is captured under CD & CE as compound 2.

### Synthesis of 1-(tert-butyl) 3-methyl 3-(4-bromobutyl)piperidine-1,3-dicarboxylate (3)

To a stirring solution of compound 2 (10 g, 0.041 mol) in THF (100 mL) was added LiHMDS (1.0 M solution in THF, 61.7 mL, 0.061 mol) drop wise at −78° C. under nitrogen atmosphere. The reaction temperature was raised to −20° C. and stirred for 1 h. Again cooled to −78° C. and 1,4-dibromobutane (7.4 mL, 0.061 mol) was added drop wise. The reaction mixture was brought to 0° C. and stirred for 3 h. After consumption of the starting material (by TLC), the reaction was quenched with saturated aqueous NH₄Cl (100 mL) and extracted with EtOAc (2×500 mL). The combined organic layer was washed with brine, dried over Na₂SO₄ and concentrated under reduced pressure. The crude material was purified by column chromatography on SiO₂ by eluting 10% EtOAc/hexane to afford compound 3 (9 g, 58%) as colorless thick liquid.

¹H NMR (500 MHz, DMSO-d₆) δ 3.77 (br d, J=13.3 Hz, 1H), 3.61 (s, 3H), 3.51 (t, J=6.7 Hz, 2H), 3.44-3.35 (m, 1H), 3.12 (br d, J=11.6 Hz, 2H), 2.00-1.89 (m, 1H), 1.80-1.69 (m, 2H), 1.55-1.40 (m, 5H), 1.38 (s, 9H), 1.31-1.21 (m, 2H).

LCMS (ESI): m/z 378.3 [M]⁺.

### Synthesis of 1-(tert-butyl) 3-methyl 3-(4-aminobutyl)piperidine-1,3-dicarboxylate (4)

To a solution of compound 3 (9 g, 0.023 mol) in methanol (90 mL) was added methanolic ammonia (7M solution, 90 mL) in sealed tube under nitrogen atmosphere. The reaction mixture was stirred at 70° C. for 16 h. After consumption of the starting material (by TLC), cooled to room temperature and volatiles were evaporated under reduced pressure. The crude was purified by column chromatography on SiO₂ by eluting with 5% MeOH/CH₂Cl₂ to afford compound 4 (5 g, 66%) as an off-white solid.

¹H NMR (500 MHz, DMSO-d₆) δ 4.08 (br d, J=4.6 Hz, 2H), 3.78 (br d, J=10.4 Hz, 1H), 3.61 (s, 3H), 3.41 (br s, 1H), 3.10 (br d, J=13.3 Hz, 2H), 2.74 (t, J=7.5 Hz, 2H), 2.01-1.89 (m, 1H), 1.54-1.41 (m, 7H), 1.38 (s, 9H), 1.25-1.10 (m, 2H).

LCMS (ESI): m/z 315.3 [M+H]⁺.

### Synthesis of tert-butyl 7-oxo-2,8-diazaspiro[5.6]dodecane-2-carboxylate (5)

To a stirring solution of compound 4 (5 g, 0.015 mol) in THF (50 mL) was added t-BuMgCl (1M solution in THF, 47.7 mL, 0.047 mol) dropwise at 0° C. The reaction mixture was stirred at room temperature for 16 h. After consumption of the starting material (by TLC), the reaction was quenched with saturated aqueous NH₄Cl (100 mL) and extracted with EtOAc (2×500 mL). The combined organic layer was washed with brine, dried over Na₂SO₄ and concentrated under reduced pressure. The crude was purified by column chromatography on SiO₂ by eluting with 60% EtOAc/hexane to afford mixture of compound 5 (3 g, 68%) as an off-white solid. Mixture of compound 5 (3 g) was separated by chiral preparative HPLC purification to obtain compound 5-F1 (1 g) as an off-white solid and compound 5-F2 (1 g) as an off-white solid.

LCMS (ESI): m/z 283.1 [M+H]⁺.

### Synthesis of 2,8-diazaspiro[5.6]dodecan-7-one (CF)

To a stirring solution of compound 5-F1 (1 g, 0.003 mol) in CH₂Cl₂ (10 mL) was added HCl (2M solution in diethyl ether, 20 mL) at 0° C. under nitrogen atmosphere. The reaction mixture was stirred at room temperature for 16 h. After consumption of the starting material (by TLC), volatiles were evaporated under reduced pressure. The crude was triturated with Et₂O and dried under reduced pressure. Obtained product was dissolved in MeOH: THF (20 mL, 1:1) and added NaHCO₃ (200 mg) portion wise at 0° C. to adjust pH to 9-10. Reaction mixture was filtered and filtrate was concentrated under reduced pressure. The crude was purified by basic alumina column chromatography by eluting 10% MeOH/CH₂Cl₂ to afford CF (300 mg) as an off-white semi solid.

### CF

¹H NMR (500 MHz, DMSO-d₆) δ 7.32 (br s, 1H), 3.19-3.08 (m, 1H), 3.03-2.92 (m, 2H), 2.69-2.59 (m, 2H), 2.23 (br d, J=12.8 Hz, 1H), 2.03 (br s, 1H), 1.75-1.58 (m, 3H), 1.52-1.21 (m, 7H)

LCMS (ESI): m/z 183.1 [+H]⁺

HPLC: 98.20%

Chiral HPLC: >99.00%

Column: CHIRALPAK IG (250*4.6 mm*5 μm)

Mobile Phase: A: 0.1% DEA in n-Hexane

Mobile Phase: B: EtOH:MeOH (50:50)

A:B: 70:30; Flow rate: 1.0 mL/min

Retention time: 35.144 min

### Synthesis of 2,8-diazaspiro[5.6]dodecan-7-one (CG)

To a stirring solution of compound 5-F2 (1 g, 0.003 mol) in CH₂Cl₂ (10 mL) was added HCl (2M solution in diethyl ether, 20 mL) at 0° C. under nitrogen atmosphere. The reaction mixture was stirred at room temperature for 16 h. After consumption of the starting material (by TLC), volatiles were evaporated under reduced pressure. The crude was triturated with Et₂O and dried under reduced pressure. Obtained product was dissolved in MeOH: THF (20 mL, 1:1) and added NaHCO₃ (200 mg) portion wise at 0° C. to adjust pH to 9-10. Reaction mixture was filtered and filtrate was concentrated under reduced pressure. The crude was purified by basic alumina column chromatography on SiO₂ by eluting 10% MeOH/CH₂Cl₂ to afford CG (300 mg) as an off-white semi solid.

### CG

¹H NMR (500 MHz, DMSO-d₆) δ 7.32 (br s, 1H), 3.21-3.08 (m, 1H), 3.05-2.89 (m, 2H), 2.66-2.58 (m, 2H), 2.23 (br d, J=12.8 Hz, 1H), 2.03 (br s, 1H), 1.72-1.58 (m, 3H), 1.53-1.21 (m, 7H)

LCMS (ESI): m/z 183.0 [M+H]⁺

HPLC: 96.95%

Chiral HPLC: >99.00%

Column: CHIRALPAK IG (250*4.6 mm*5 μm)

Mobile Phase: A: 0.1% DEA in n-Hexane

Mobile Phase: B: EtOH:MeOH (50:50)

A:B: 70:30; Flow rate: 1.0 mL/min

Retention time: 28.833 min

### Synthetic Scheme for AX & AY

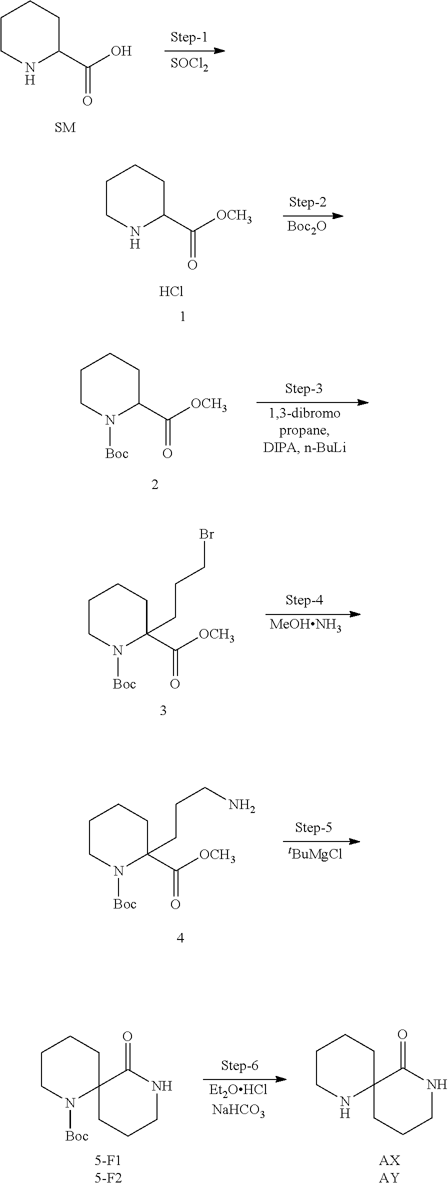

### Synthesis of methyl piperidine-2-carboxylate hydrochloride (1)

To a stirring solution of piperidine-2-carboxylic acid (SM) (100 g, 0.775 mol) in methanol (800 mL) was added thionyl chloride (115 mL, 1.55 mol) drop wise at 0° C. under nitrogen atmosphere. The reaction mixture was stirred at room temperature for 16 h. After consumption of the starting material (by TLC), volatiles were concentrated under reduced pressure. The crude was triturated with hexane and dried under vacuum to afford crude compound 1 (140 g) as colorless liquid. This product was taken to next step without any further purification.

¹H NMR (500 MHz, DMSO-d₆) δ 9.84 (br s, 1H), 9.37 (br s, 1H), 4.06 (br s, 1H), 3.75 (s, 3H), 3.21 (br d, J=12.4 Hz, 1H), 2.88 (br d, J=7.7 Hz, 1H), 2.06 (br d, J=12.0 Hz, 1H), 1.78-1.61 (m, 4H), 1.60-1.47 (m, 1H).

LCMS (ESI): m/z 144.0 [M+H]⁺.

### Synthesis of 1-(tert-butyl) 2-methyl piperidine-1,2-dicarboxylate (2)

To a stirring solution of compound 1 (70 g, 0.389 mol) in CH₂Cl₂ (1 L) was added Et₃N (140 mL, 0.972 mol) at 0° C. slowly and stirred for 15 min. Boc₂O (107 mL, 0.467 mol) was added at 0° C. and the reaction mixture was stirred at room temperature for 16 h. After consumption of the starting material (by TLC), the reaction was diluted with CH₂Cl₂ (2 L) and washed with water (1 L) and brine (1 L). Organic layer was dried over Na₂SO₄ and concentrated under reduced pressure to afford compound 2 (80 g, 84%) as colorless liquid.

¹H NMR (400 MHz, DMSO-d₆) δ 4.79-4.57 (m, 1H), 3.81 (br d, J=12.5 Hz, 1H), 3.67 (s, 3H), 2.98-2.69 (m, 1H), 2.04 (br s, 1H), 1.64-1.57 (m, 3H), 1.39, 1.36 (2s, 9H), 1.33-1.24 (m, 1H), 1.17-1.04 (m, 1H).

LCMS (ESI): m/z 144.0 [M-Boc+H]⁺.

### Synthesis of 1-(tert-butyl) 2-methyl 2-(3-bromopropyl)piperidine-1,2-dicarboxylate (3)

To a stirring solution of DIPA (36 mL, 0.257 mol) in THF (250 mL) was added n-BuLi (117 mL, 0.257 mol) drop wise at −78° C. under nitrogen atmosphere. The reaction mixture was stirred at −10° C. for 1 h. Again cooled to −78° C., compound 2 (25 g, 0.102 mol) was added and stirred at −30° C. for 1 h. Again cooled to −78° C., 1,3-dibromopropane (21 mL, 0.205 mol) was added. The reaction mixture was brought to room temperature and stirred for 16 h. After consumption of the starting material (by TLC), the reaction was quenched with saturated aqueous NH₄Cl (100 mL) and extracted with EtOAc (2×500 mL). The combined organic layer was washed with brine, dried over Na₂SO₄ and concentrated under reduced pressure. The crude material was purified by column chromatography on SiO₂ by eluting 10% EtOAc/hexane to afford compound 3 (10 g, 27%) as colorless liquid.

LCMS (ESI): m/z 266.2 [M-Boc+H]⁺.

### Synthesis of 1-(tert-butyl) 2-methyl 2-(3-aminopropyl)piperidine-1,2-dicarboxylate (4)

To a solution of compound 3 (10 g, 0.027 mol) in methanol (30 mL) was added methanolic ammonia (7M solution, 100 mL) in sealed tube under nitrogen atmosphere. The reaction mixture was stirred at 50° C. for 16 h. After consumption of the starting material (by TLC), cooled to room temperature and volatiles were evaporated under reduced pressure. The crude was purified by column chromatography on SiO₂ by eluting with 2% MeOH/CH₂Cl₂ to afford compound 4 (2.6 g, 31%) as sticky solid.

¹H NMR (400 MHz, DMSO-d₆) δ 7.78 (br s, 2H), 3.75 (br d, J=12.5 Hz, 1H), 3.61 (s, 3H), 3.04-2.89 (m, 1H), 2.78 (t, J=7.6 Hz, 2H), 2.04-1.78 (m, 2H), 1.65-1.47 (m, 8H), 1.35 (s, 9H).

LCMS (ESI): m/z 300.2 [M]⁺.

### Synthesis of tert-butyl 7-oxo-1,8-diazaspiro[5.5]undecane-1-carboxylate (5)

To a stirring solution of compound 4 (2.6 g, 8.66 mmol) in THF (26 mL) was added t-BuMgCl (1M solution in THF, 43.6 mL, 43.6 mmol) dropwise at 0° C. and the reaction mixture was stirred at room temperature for 16 h. After consumption of the starting material (by TLC), the reaction was quenched with saturated aqueous NH₄Cl (100 mL) and extracted with EtOAc (2×500 mL). The combined organic layer was washed with brine, dried over Na₂SO₄ and concentrated under reduced pressure. The crude was purified by column chromatography on SiO₂ by eluting with 2% 10% MeOH/CH₂Cl₂ to afford mixture of compound 5 (1.5 g, 65%) as pale yellow sticky solid. Mixture of compound 5 (1.5 g) was separated by chiral preparative HPLC purification to obtain compound 5-F1 (640 mg) as an off-white solid and compound 5-F2 (604 g) as an off-white solid.

### Compound 5-F1

¹H NMR (400 MHz, DMSO-d₆) δ 7.24 (br s, 1H), 3.75-3.53 (m, 1H), 3.22-2.95 (m, 3H), 2.03 (br d, J=12.5 Hz, 1H), 1.93-1.64 (m, 5H), 1.61-1.39 (m, 4H), 1.36 (s, 9H).

LCMS (ESI): m/z 269.1 [M+H]⁺.

### Compound 5-F2

¹H NMR (400 MHz, DMSO-d₆) δ 7.23 (br s, 1H), 3.74-3.53 (m, 1H), 3.24-2.96 (m, 3H), 2.03 (br d, J=12.8 Hz, 1H), 1.91-1.64 (m, 5H), 1.62-1.41 (m, 4H), 1.36 (s, 9H).

LCMS (ESI): m/z 537.4 [2M+H]⁺.

### Synthesis of 1,8-diazaspiro[5.5]undecan-7-one (AX)

To a stirring solution of compound 5-F1 (640 mg, 2.38 mol) in CH₂Cl₂ (2.5 mL) was added HCl (2M solution in diethyl ether, 12 mL) at 0° C. under nitrogen atmosphere. The reaction mixture was stirred at room temperature for 4 h. After consumption of the starting material (by TLC), volatiles were evaporated under reduced pressure. The crude was triturated with Et₂O and dried under reduced pressure. Obtained product was dissolved in MeOH: THF (10 mL, 1:1) and added NaHCO₃ (741 mg, 8.82 mmol) portion wise at 0° C. to adjust pH to 9-10. Reaction mixture was filtered and filtrate was concentrated under reduced pressure. The crude was purified by basic alumina column chromatography on SiO₂ by eluting 10% MeOH/CH₂Cl₂ to afford AX (206 mg) as an off-white solid.

### AX

¹H NMR (400 MHz, DMSO-d₆) δ 7.29 (br s, 1H), 3.16-2.97 (m, 2H), 2.83-2.73 (m, 1H), 2.62-2.53 (m, 1H), 2.00-1.82 (m, 2H), 1.79-1.37 (m, 8H), 1.34-1.19 (m, 1H)

LCMS (ESI): m/z 169.0 [M+H]⁺

HPLC: 99.47%

Chiral HPLC: >99.00%

Column: CHIRALPAK IC (250*4.6 mm*5 μm)

Mobile Phase: A: 0.1% DEA in n-Hexane

Mobile Phase: B: DCM:MeOH (50:50)

A:B: 75:25; Flow rate: 1.0 mL/min

Retention time: 16.099 min

### Synthesis of 1,8-diazaspiro[5.5]undecan-7-one (AY)

To a stirring solution of compound 5-F2 (604 mg, 2.25 mol) in CH₂Cl₂ (2.5 mL) was added HCl (2M solution in diethyl ether, 12 mL) at 0° C. under nitrogen atmosphere. The reaction mixture was stirred at room temperature for 4 h. After consumption of the starting material (by TLC), volatiles were evaporated under reduced pressure. The crude was triturated with Et₂O and dried under reduced pressure. Obtained product was dissolved in MeOH: THF (10 mL, 1:1) and added NaHCO₃ (992 mg, 11.8 mmol) portion wise at 0° C. to adjust pH to 9-10. Reaction mixture was filtered and filtrate was concentrated under reduced pressure. The crude was purified by basic alumina column chromatography on SiO₂ by eluting 10% MeOH/CH₂Cl₂ to afford AY (306 mg) as an off-white solid.

### AY

¹H NMR (400 MHz, DMSO-d₆) δ 7.29 (br s, 1H), 3.18-2.98 (m, 2H), 2.82-2.75 (m, 1H), 2.61-2.53 (m, 1H), 1.96-1.81 (m, 2H), 1.78-1.37 (m, 8H), 1.33-1.19 (m, 1H)

LCMS (ESI): m/z 169.1 [M+H]⁺

HPLC: 98.87%

Chiral HPLC: >99.00%

Column: CHIRALPAK IC (250*4.6 mm*5 μm)

Mobile Phase: A: 0.1% DEA in n-Hexane

Mobile Phase: B: DCM:MeOH (50:50)

A:B: 75:25; Flow rate: 1.0 mL/min

Retention time: 11.221 min

### Synthetic Scheme for AZ & BA

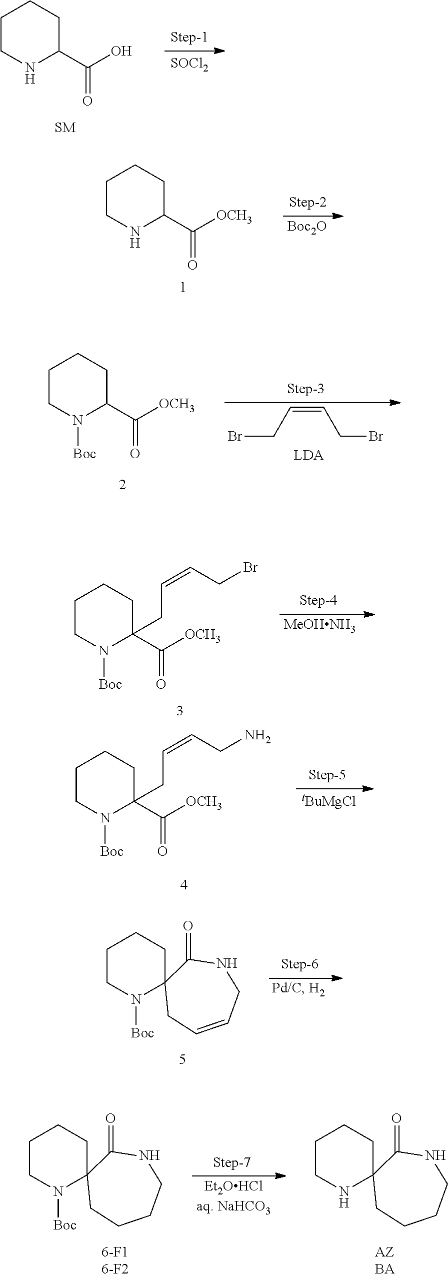

The experimental procedure for the synthesis of compound 2 is captured under AX & AY as compound 2.

### Synthesis of 1-(tert-butyl) 2-methyl (Z)-2-(4-bromobut-2-en-1-yl)piperidine-1,2-dicarboxylate (3)

To a solution of compound 2 (24 g, 0.1 mol) in THF (240 mL) was added LDA (1M in THF, 150 mL, 0.15 mol) drop wise at −78° C. and stirred for 1 h. (Z)-1,4-dibromobut-2-ene (32 g, 0.15 mol) was added drop wise. The reaction mixture was stirred at room temperature for 16 h. After consumption of the starting material (by TLC), the reaction was quenched with saturated aqueous NH₄Cl (1 L) and extracted with EtOAc (3×50 mL). The combined organic layer was dried over Na₂SO₄ and concentrated under reduced pressure. The crude material was purified by column chromatography on SiO₂ by eluting with 15% EtOAc/hexane to afford compound 3 (13 g, 35%) as pale yellow liquid.

¹H NMR (400 MHz, DMSO-d₆) δ 5.85-5.76 (m, 1H), 5.72-5.62 (m, 1H), 3.76-3.65 (m, 2H), 3.63 (s, 3H), 3.11-2.96 (m, 1H), 2.89-2.79 (m, 1H), 2.76-2.63 (m, 1H), 1.86-1.45 (m, 7H), 1.35 (s, 9H).

LCMS (ESI): m/z 277.0 [M-Boc+H]⁺.

### Synthesis of 1-(tert-butyl) 2-methyl (Z)-2-(4-aminobut-2-en-1-yl)piperidine-1,2-dicarboxylate (4)

To a solution of compound 3 (11.5 g, 0.031 mol) in methanol (25 mL) was added methanolic ammonia (7M solution, 100 mL) in sealed tube under nitrogen atmosphere. The reaction mixture was stirred at room temperature for 24 h. After consumption of the starting material (by TLC), volatiles were evaporated under reduced pressure. The crude was purified by column chromatography on SiO₂ by eluting with 6% MeOH/CH₂Cl₂ to afford compound 4 (7 g, 73%) as pale yellow liquid.

¹H NMR (500 MHz, DMSO-d₆) δ 7.83 (br s, 2H), 5.81-5.67 (m, 1H), 5.64-5.38 (m, 1H), 3.71 (br d, J=11.6 Hz, 1H), 3.62 (s, 3H), 3.58-3.40 (m, 2H), 3.01 (br s, 1H), 2.82-2.78 (m, 1H), 2.65-2.58 (m, 1H), 1.87-1.43 (m, 6H), 1.35 (s, 9H).

LCMS (ESI): m/z 313.3 [M+H]⁺.

### Synthesis of tert-butyl 7-oxo-1,8-diazaspiro[5.6]dodec-10-ene-1-carboxylate (5)

To a stirring solution of compound 4 (7 g, 0.022 mol) in THF (20 mL) was added t-BuMgCl (1M solution in THF, 112 mL, 0.112 mol) dropwise at 0° C. The reaction mixture was stirred at room temperature for 16 h. After consumption of the starting material (by TLC), the reaction was quenched with saturated aqueous NH₄Cl (100 mL) at 0° C. and extracted with EtOAc (2×500 mL). The combined organic layer was washed with brine, dried over Na₂SO₄ and concentrated under reduced pressure. The crude was purified by column chromatography on SiO₂ by eluting with 2% MeOH/CH₂Cl₂ to afford mixture of compound 5 (4.2 g, 67%) as pale yellow solid.

¹H NMR (400 MHz, DMSO-d₆) δ 7.31 (br d, J=4.4 Hz, 1H), 6.00-5.66 (m, 2H), 3.78 (br d, J=16.7 Hz, 1H), 3.52 (br s, 1H), 3.30-3.21 (m, 1H), 3.10-2.90 (m, 2H), 2.18 (br d, J=16.1 Hz, 1H), 1.83-1.68 (m, 1H), 1.66-1.46 (m, 4H), 1.42-1.32 (m, 10H).

LCMS (ESI): m/z 181.2 [M-Boc+H]⁺.

### Synthesis of tert-butyl 7-oxo-1,8-diazaspiro[5.6]dodecane-1-carboxylate (6)

To a stirring solution of compound 5 (2.5 g, 8.92 mmol) in MeOH (50 mL) was added 10% Pd/C (50% wet, 2 g) at room temperature and stirred under H₂ atmosphere (balloon pressure) for 16 h. After consumption of the starting material (by TLC), the reaction mixture was filtered through a pad of celite and the pad was washed with MeOH (100 mL). The filtrate was concentrated and dried under vacuum to afford mixture compound 6 (1.4 g, 56%) as an off white solid. Mixture of compound 6 (1.4 g) was separated by chiral preparative HPLC purification to obtain compound 6-F1 (620 mg) as an off-white solid and compound 6-F2 (620 g) as an off-white solid.

### Compound 6-F1

¹H NMR (400 MHz, DMSO-d₆) δ 7.20 (br s, 1H), 3.66-3.60 (m, 1H), 3.22-3.08 (m, 1H), 3.04-2.80 (m, 2H), 2.44-2.34 (m, 1H), 1.90-1.73 (m, 1H), 1.69-1.40 (m, 10H), 1.37 (s, 9H)

LCMS (ESI): m/z 565.4 [2M+H]⁺.

### Compound 6-F2

¹H NMR (400 MHz, DMSO-d₆) δ 7.20 (br s, 1H), 3.66-3.60 (m, 1H), 3.25-3.07 (m, 1H), 3.03-2.79 (m, 2H), 2.44-2.33 (m, 1H), 1.91-1.74 (m, 1H), 1.68-1.41 (m, 10H), 1.37 (s, 9H)

LCMS (ESI): m/z 565.5 [2M+H]⁺.

### Synthesis of 1,8-diazaspiro[5.6]dodecan-7-one (AZ)

To a stirring solution of compound 6-F1 (520 mg, 1.84 mmol) in CH₂Cl₂ (2.5 mL) was added HCl (2M solution in diethyl ether, 10 mL) at 0° C. under nitrogen atmosphere. The reaction mixture was stirred at room temperature for 4 h. After consumption of the starting material (by TLC), volatiles were evaporated under reduced pressure. The crude was triturated with Et₂O and dried under reduced pressure. Obtained product was dissolved in MeOH: THF (10 mL, 1:1) and added NaHCO₃ (809 mg, 9.63 mmol) portion wise at 0° C. to adjust pH to 9-10. Reaction mixture was filtered and filtrate was concentrated under reduced pressure. The crude was purified by basic alumina column chromatography on SiO₂ by eluting with 5% MeOH/CH₂Cl₂ to afford AZ (312 mg) as pale brown liquid.

### AZ

¹H NMR (400 MHz, DMSO-d₆) δ 7.23 (br s, 1H), 3.72-3.50 (m, 1H), 2.97-2.80 (m, 1H), 2.79-2.69 (m, 1H), 2.00-1.75 (m, 3H), 1.73-1.40 (m, 6H), 1.38-1.19 (m, 4H), 1.15-1.02 (m, 1H)

LCMS (ESI): m/z 183.0 [M+H]⁺

HPLC: 99.82%

Chiral HPLC: 96.71%

Column: CHIRALPAK IC (250*4.6 mm*5 μm)

Mobile Phase: A: 0.1% DEA in n-Hexane

Mobile Phase: B: DCM:MeOH (50:50)

A:B: 75:25; Flow rate: 1.0 mL/min

Retention time: 8.826 min

### Synthesis of 1,8-diazaspiro[5.6]dodecan-7-one (BA)

To a stirring solution of compound 6-F2 (520 mg, 1.84 mmol) in CH₂Cl₂ (2.5 mL) was added HCl (2M solution in diethyl ether, 10 mL) at 0° C. under nitrogen atmosphere. The reaction mixture was stirred at room temperature for 4 h. After consumption of the starting material (by TLC), volatiles were evaporated under reduced pressure. The crude was triturated with Et₂O and dried under reduced pressure. Obtained product was dissolved in MeOH: THF (10 mL, 1:1) and added NaHCO₃ (809 mg, 9.63 mmol) portion wise at 0° C. to adjust pH to 9-10. Reaction mixture was filtered and filtrate was concentrated under reduced pressure. The crude was purified by basic alumina column chromatography by eluting 5% MeOH/CH₂Cl₂ to afford BA (295 mg) as pale brown semi solid.

### BA

¹H NMR (400 MHz, DMSO-d₆) δ 7.26 (br s, 1H), 3.70-3.51 (m, 1H), 2.98-2.82 (m, 1H), 2.80-2.68 (m, 1H), 2.59-2.52 (m, 1H), 1.98-1.75 (m, 2H), 1.73-1.43 (m, 6H), 1.40-1.21 (m, 4H), 1.18-1.02 (m, 1H)

LCMS (ESI): m/z 183.0 [M+H]⁺

HPLC: 99.26%

Chiral HPLC: 95.10%

Column: CHIRALPAK IC (250*4.6 mm*5 μm)

Mobile Phase: A: 0.1% DEA in n-Hexane

Mobile Phase: B: DCM:MeOH (50:50)

A:B: 75:25; Flow rate: 1.0 mL/min

Retention time: 13.346 min

### Synthetic Scheme for GA & AU-1

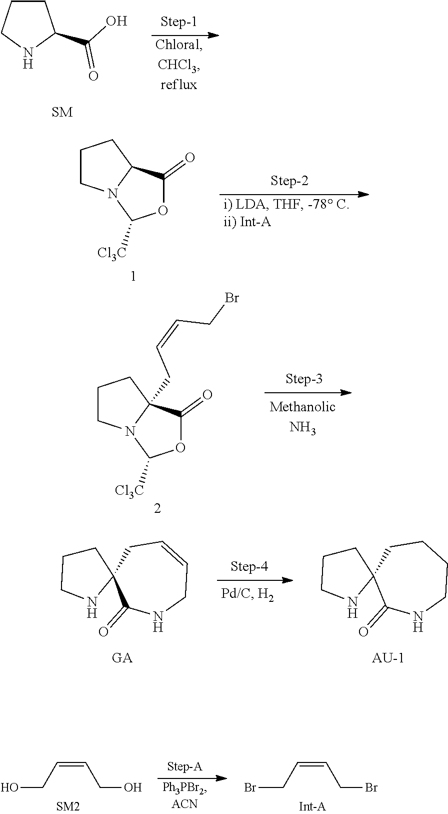

### Synthesis of (3R,7aS)-3-(trichloromethyl)tetrahydro-1H,3H-pyrrolo[1,2-c]oxazol-1-one (1)

To a stirring solution of compound SM-1 (100 g, 0.869 mol) in chloroform (1000 mL), chloral (172.1 g, 1.04 mol) was added and reaction mixture was heated at 65° C. for 16 h (using dean-stark apparatus). After consumption of the starting material (by TLC), the reaction mixture was concentrated under reduced pressure. The residue on recrystallization with ethanol afforded compound 1 (100 g, 47%) as a white solid.

¹H NMR (400 MHz, DMSO-d₆) δ 5.23 (s, 1H), 4.11-4.08 (m, 1H), 3.43-3.37 (m, 1H), 3.13-3.07 (m, 1H), 2.20-2.18 (m, 1H), 2.11-2.08 (m, 1H), 1.92-1.88 (m, 1H), 1.75-1.70 (m, 1H).

### Synthesis of (3R,7aR)-7a-((Z)-4-bromobut-2-en-1-yl)-3-(trichloromethyl)tetrahydro-1H,3H-pyrrolo[1,2-c]oxazol-1-one (2)

To a stirred solution of compound 1 (40.0 g, 0.163 mol) in THF (400 mL), LDA (2M solution in THF, 122.9 mL, 0.245 mol) was added at −78° C. and stirred at same temperature for 20 min. Int-A (69.8 g, 0.327 mmol) was added dropwise to the reaction mixture at −78° C. and stirred at same temperature for 4 h. After consumption of the starting material (by TLC), the reaction mixture was quenched with water (300 mL) and extracted with EtOAc (3×400 mL). The combined organic layer was washed with brine (200 mL), dried over Na₂SO₄ and concentrated under reduced pressure. The residue was purified by flash column chromatography on SiO₂ to afford compound 2 (30 g, 48%) as an oil.

¹H NMR (400 MHz, DMSO-d₆) δ 6.01-5.94 (m, 1H), 5.80-5.40 (m, 1H), 5.01 (s, 1H), 4.09-4.04 (m, 1H), 4.0-3.96 (m, 2H), 3.26-3.20 (m, 2H), 2.80-2.59 (m, 2H), 2.26-2.16 (m, 1H), 2.06-1.90 (m, 2H).

### Synthesis of (R)-1,7-diazaspiro[4.6]undec-9-en-6-one (GA)

To a stirred solution of compound 2 (15 g, 0.039 mol) in MeOH (20 mL), methanolic ammonia (100 mL) was added at 0° C. under nitrogen atmosphere and stirred at room temperature for 16 h. After consumption of the starting material (by TLC), and the reaction mixture was concentrated under reduced pressure. The residue was dissolved in aqueous 2M HCl, washed with ethyl acetate. The aqueous layer was basified (pH-12) by the addition of solid NaOH and extracted with dichloromethane. The organic layer was dried over Na₂SO₄ and concentrated under reduced pressure. The residue was purified by column chromatography to afford compound GA (3.0 g, 45%) as a pale yellow solid.

¹H NMR (400 MHz, DMSO-d₆) δ 7.65 (s, 1H), 5.70-5.54 (m, 2H), 3.80-3.59 (m, 2H), 3.26-3.14 (m, 1H), 2.76 (d, J=6.9 Hz, 2H), 2.21-2.00 (m, 3H), 1.78-1.52 (m, 3H).

LCMS (ESI): m/z 167 [M+H]⁺.

HPLC: 95.4%.

### Synthesis of (S)-1,7-diazaspiro[4.6]undecan-6-one (AU-1)

To a stirring solution of compound GA (0.5 g, 3.01 mmol) in MeOH (20 mL) and EtOAc (10 mL), 10% Pd/C (50% wet, 50 mg) was added at room temperature and stirred under H₂ atmosphere (balloon) for 12 h. After consumption of the starting material (by TLC), the reaction mixture was filtered through a pad of celite and washed with MeOH (50 mL). The filtrate was concentrated under reduced pressure. The residue was purified by combiflash chromatography to afford compound AU-1 (200 mg, 40%) as an off white solid.

¹H NMR (400 MHz, DMSO-d₆) δ 7.54 (brs, 1H), 3.25-3.03 (m, 2H), 3.02-2.99 (m, 1H), 2.85-2.80 (m, 1H), 2.64-2.58 (m, 1H), 1.98-1.91 (m, 1H), 1.77-1.47 (m, 8H), 1.45-1.23 (m, 1H).

LCMS (ESI): m/z 169 [M+H]⁺.

HPLC: 97.41%.

### Synthesis of (Z)-1,4-dibromobut-2-ene (A)

To a stirred solution of compound triphenylphosphane (100 g, 0.381 mol) in ACN (500 mL), bromine (19 mL, 0.381 mol) was added dropwise at 0° C. and stirred at same temperature for 1 h. After that (Z)-but-2-ene-1,4-diol (15 g, 0.381 mol) was added and reaction mixture was heated at 50° C. for 4 h. After consumption of the starting material (by TLC), the reaction mixture was quenched with water (300 mL) and extracted with Et₂O (3×300 mL). The combined organic layer was washed with brine (100 mL), dried over Na₂SO₄ and concentrated under reduced pressure to afford compound A (26 g, crude) as thick oil.

¹H NMR (400 MHz, DMSO-d₆) δ 6.03-5.86 (m, 2H), 4.06-3.95 (m, 4H).

### Synthetic Scheme for EV-1, EV-2, EU-1 & EU-2

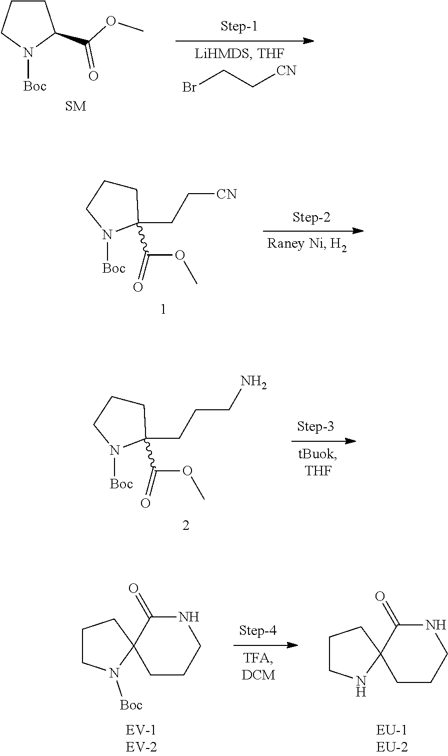 [SMILES: COC(=O)[C@@H]1CCCN1C(=O)OC(C)(C)C ; N#CCCBr ; COC(=O)C1(CCC#N)CCCN1C(=O)OC(C)(C)C ; COC(=O)C1(CCCN)CCCN1C(=O)OC(C)(C)C ; CC(C)(C)OC(=O)N1CCCC12CCCNC2=O ; O=C1NCCCC12CCCN2]

### Synthesis of 1-(tert-butyl) 2-methyl 2-(2-cyanoethyl)pyrrolidine-1,2-dicarboxylate (1)

To a stirred solution of 1-(tert-butyl) 2-methyl (S)-pyrrolidine-1,2-dicarboxylate (SM) (10.0 g, 46.7 mmol) in THF (150 mL), LiHMDS (1M solution in THF, 70 mL, 70.0 mmol) was added at −78° C. and 3-bromopropanenitrile (6.8 mL, 51.4 mmol) was added dropwise and stirred at room temperature for 16 h. After consumption of the starting material (by TLC), the reaction mixture was quenched with saturated NH₄Cl solution (300 mL) and extracted with EtOAc (3×300 mL). The combined organic layer was washed with brine (100 mL), dried over Na₂SO₄ and concentrated under reduced pressure. The residue was purified by column chromatography on SiO₂ to afford compound 1 (6.0 g, 37%) as thick oil.

¹H NMR (400 MHz, DMSO-d₆) δ 3.69 (s, 3H), 3.44 (dd, J=10.8, 5.8 Hz, 4H), 3.33-3.28 (m, 2H), 2.19-1.85 (m, 4H), 1.39 (s, 9H).

### Synthesis of 1-(tert-butyl) 2-methyl 2-(3-aminopropyl)pyrrolidine-1,2-dicarboxylate (2)

To a stirring solution of compound 1 (15.0 g, 55.9 mmol) in MeOH (100 mL) and THF (100 mL), Raney Nickel (7.05 g, 167 mmol) was added at room temperature and stirred under H₂ atmosphere at 50° C. for 56 h. After consumption of the starting material (by TLC), the reaction mixture was filtered through a pad of celite and washed with MeOH (50 mL). The filtrate was concentrated under reduced pressure to afford compound 2 (15.0 g, crude) as a thick oil.

¹H NMR (400 MHz, DMSO-d₆) δ 4.22-4.06 (m, 1H), 3.66-3.43 (m, 2H), 3.42-3.22 (m, 3H), 3.16 (s, 3H), 2.79-2.70 (m, 1H), 2.53-2.46 (m, 1H), 2.01-1.97 (m, 1H), 1.81-1.74 (m, 1H), 1.69-1.57 (m, 1H), 1.39-1.35 (m, 3H), 1.16 (s, 9H).

### Synthesis of tert-butyl 6-oxo-1,7-diazaspiro[4.5]decane-1-carboxylate (EV-1 & EV-2)

To a stirred solution of compound 3 (8.0 g, 27.9 mmol) in THF (100 mL), t-BuOK (8.0 g, 27.9 mmol) was added at 0° C. and stirred for 15 minutes. The reaction mixture was stirred at room temperature for 5 h. After consumption of the starting material (by TLC), the reaction mixture was quenched with NH₄Cl solution (150 mL) and extracted with EtOAc (2×200 mL). The combined organic layer was washed with brine (50 mL), dried over Na₂SO₄ and concentrated under reduced pressure. The residue was purified by column chromatography on SiO₂ to afford mixture of compounds EV-1 & EV-2 (0.8 g) as a colorless solid. The mixture was purified by preparative HPLC followed by chiral HPLC to afford EV-1 (220 mg) as a colorless solid and EV-2 (220 mg) as a colorless solid.

### EV-1

¹H NMR (400 MHz, DMSO-d₆) δ 7.56 (s, 1H), 7.40-7.35 (m, 1H), 3.38 (dd, J=8.5, 5.0 Hz, 1H), 3.27-3.20 (m, 2H), 3.20-3.05 (m, 5H), 2.19-2.07 (m, 3H), 1.35 and 1.34 (2s, 9H).

HPLC: 97.32%.

### EV-2

: δ 7.54 (s, 1H), 7.40-7.35 (m, 1H), 3.38 (dd, J=8.5, 5.0 Hz, 1H), 3.27-3.20 (m, 1H), 3.20-3.05 (m 2H), 2.19-1.61 (m 8H), 1.35 and 1.34 (2s, 9H).

HPLC: 95.94%.

### Synthesis of 1,7-diazaspiro[4.5]decan-6-one (EU-1)

To a stirred solution of EV-1 (0.22 g, 0.87 mmol) in CH₂Cl₂ (5 mL), TFA (0.15 mL, 1.04 mmol) was added at 0° C. and the reaction mixture was stirred at room temperature for 3 h. After consumption of the starting material (by TLC), the reaction mixture concentrated under reduced pressure and the crude obtained was quenched with DIPEA. The crude was purified by column chromatography on SiO₂ to afford EU-1 (0.10 g, 75%).

¹H NMR (400 MHz, DMSO-d₆) δ 8.76 (br s, 1H), 8.11 (s, 1H), 3.23-3.17 (m, 4H), 2.05-1.74 (m, 8H).

LCMS (ESI) m/z=154.95 [M+H]⁺.

HPLC: 97.48%.

### Synthesis of 1,7-diazaspiro[4.5]decan-6-one (EU-2)

To a stirred solution of EV-2 (0.22 g, 0.87 mmol) in CH₂Cl₂ (5 mL), TFA (0.11 mL, 1.04 mmol) was added at 0° C. and the reaction mixture was stirred at room temperature for 3 h. After consumption of the starting material (by TLC), the reaction mixture concentrated under reduced pressure and the crude obtained was quenched with DIPEA. The crude was purified by column chromatography to afford EU-2 (0.08 g, 60%).

¹H NMR (400 MHz, DMSO-d₆) δ 8.14 (br s, 1H), 7.98 (s, 1H), 3.32-3.12 (m, 6H), 2.03-1.76 (m, 6H).

LCMS (ESI) m/z=154.85 [M+H]⁺.

HPLC: 92.48%.

### Synthetic Scheme for AU-2

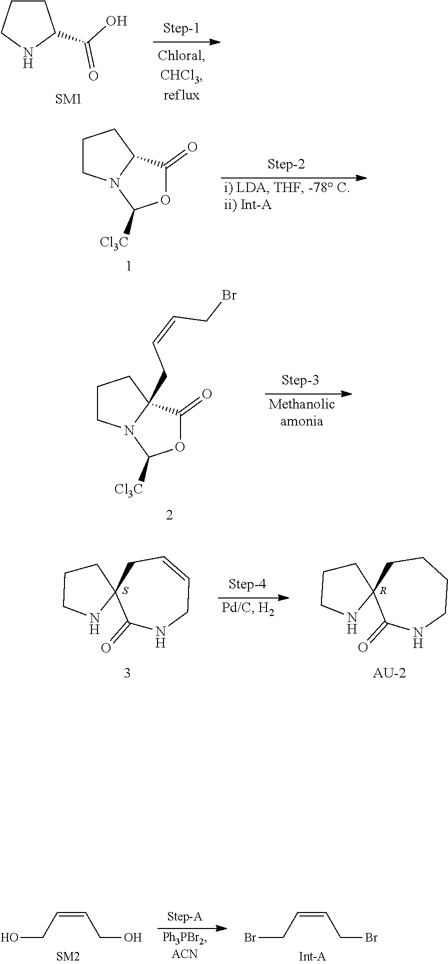

### Synthesis of (3S,7aR)-3-(trichloromethyl)tetrahydro-1H,3H-pyrrolo[1,2-c]oxazol-1-one (1)

To a stirring solution of D-Proline (SM1) (5 g, 43.4 mmol) in chloroform (100 mL), chloral (8.5 g, 52.1 mmol) was added and reaction mixture was heated at 65° C. for 16 h (using dean-stark apparatus). After consumption of the starting material (by TLC), the reaction mixture was concentrated under reduced pressure. Recrystallization in ethanol afforded compound 1 (4 g, 38%) as a white solid.

¹H NMR (400 MHz, DMSO-d₆) δ 5.16 (s, 1H), 4.18-4.10 (m, 1H), 3.45-3.39 (m, 1H), 3.15-3.09 (m, 1H), 2.27-2.18 (m, 1H), 2.12-2.08 (m, 1H), 1.97-1.92 (m, 1H), 1.79-1.73 (m, 1H).

### Synthesis of (3S,7aS)-7a-((Z)-4-bromobut-2-en-1-yl)-3-(trichloromethyl)tetrahydro-1H,3H-pyrrolo[1,2-c]oxazol-1-one (2)

To a stirred solution of compound 1 (3.7 g, 15.13 mmol) in THF (40 mL), LDA (2M solution in THF, 22.6 mL, 22.6 mmol) was added at −78° C. and stirred at same temperature for 20 min. To the reaction mixture, Int-A (4.7 g, 22.6 mmol) was added dropwise at −78° C. and stirred at same temperature for 4 h. After consumption of the starting material (by TLC), the reaction mixture was quenched with water (300 mL) and extracted with EtOAc (3×200 mL). The combined organic layer was washed with brine (100 mL), dried over Na₂SO₄ and concentrated under reduced pressure. The residue was purified by flash column chromatography on SiO₂ to afford compound 2 (3.8 g, 67.8%) as thick oil.

LCMS (ESI): m/z 376 [M+H]⁺.

### Synthesis of (S)-1,7-diazaspiro[4.6]undec-9-en-6-one (3)

To a stirred solution of compound 2 (3.5 g, 9.35 mmol) in MeOH (20 mL), methanolic ammonia (20 mL) was added at 0° C. under nitrogen atmosphere and stirred at room temperature for 16 h. After consumption of the starting material (by TLC), and then evaporated to give a residue which was dissolved in 2M HCl. The acidic layer was washed with ethyl acetate and then made basic (pH 12) by the addition of solid NaOH. Extraction with dichloromethane and dried over Na₂SO₄ and concentrated under reduced pressure. The residue was purified by column chromatography on SiO₂ to afford compound 3 (0.3 g, 20%) as a pale yellow semisolid.

LCMS (ESI): m/z 167 [M+H]⁺.

### Synthesis of (R)-1,7-diazaspiro[4.6]undecan-6-one (AU-2)

To a stirring solution of compound 3 (0.15 g, 0.9 mmol) in MeOH (2 mL) and EtOAc (2 mL), 10% Pd/C (20 mg) was added at room temperature and stirred under H₂ atmosphere (balloon) for 4 h. After consumption of the starting material (by TLC), the reaction mixture was filtered through a pad of celite and washed with MeOH (50 mL). The filtrate was concentrated under reduced pressure. The residue was purified by flash chromatography to afford compound AU-2 (120 mg, 80%) as an off white solid.

¹H NMR (400 MHz, DMSO-d₆) δ 7.89 (brs, 1H), 3.10-3.03 (m, 2H), 3.02-2.99 (m, 1H), 2.86-2.80 (m, 1H), 2.08-2.01 (m, 1H), 1.93-1.90 (m, 1H), 1.81-1.54 (m, 8H), 1.40-1.23 (m, 1H).

LCMS (ESI): m/z 169 [M+H]⁺.

HPLC: 95.08%.

### Synthesis of (Z)-1,4-dibromobut-2-ene (Int-A)

To a stirring solution of triphenylphosphine (100 g, 0.381 mol) in acetonitrile (500 mL), bromine (19 mL, 0.381 mol) was added dropwise at 0° C. and stirred at same temperature for 1 h. After that (Z)-but-2-ene-1,4-diol (SM-2) (15 g) was added and reaction mixture was heated at 50° C. for 4 h. After consumption of the starting material (by TLC), the reaction mixture was quenched with water (300 mL) and extracted with Et₂O (3×300 mL). The combined organic layer was washed with brine (100 mL), dried over Na₂SO₄ and concentrated under reduced pressure to afford Int-A (26 g, crude) as thick oil.

¹H NMR (400 MHz, DMSO-d₆) δ 6.03-5.86 (m, 2H), 4.06-3.95 (m, 4H).

### Synthetic Scheme for EI-1 & EI-2

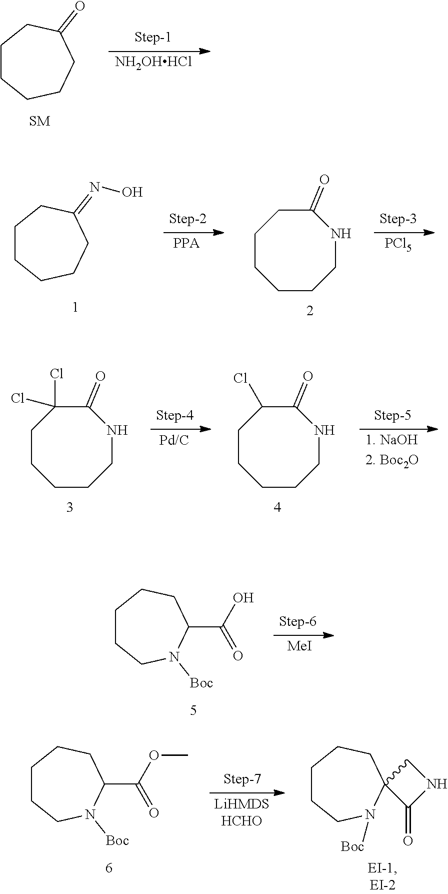

### Synthesis of Cycloheptanone Oxime (1)

To a stirred solution of cycloheptanone (SM) (20 g, 178.3 mmol) in ethanol (200 mL) was added hydroxylamine hydrochloride (14.9 g, 213.9 mmol) and then heated to reflux for 1 h. After consumption of the starting material (by TLC), the reaction mixture was brought to room temperature and volatiles were evaporated under reduced pressure. Crude material was diluted with water (200 mL) and extracted with EtOAc (2×200 mL). Combined organic layer was dried over Na₂SO₄ and concentrated under reduced pressure to obtain compound 1 (15.5 g, 68%) as off white solid, which was taken next step without any further purification.

¹H-NMR: (500 MHz, DMSO-d₆): δ 10.24 (br s, 1H), 2.40 (t, J=5.5 Hz, 2H), 2.28 (t, J=5.5 Hz, 2H), 1.60-1.40 (m, 8H).

LCMS (m/z): 128 [M+H]⁺.

### Synthesis of azocan-2-one (2)

To a solution of compound 1 (10.5 g, 82.5 mmol) in o-xylene (63 mL) was added polyphosphoric acid (15 mL). The reaction mixture was heated to 120° C. and stirred for 1 h. After consumption of the starting material (by TLC), the reaction mixture was brought to room temperature and o-xylene was removed by decantation. Crude material was diluted with cold water (20 mL) and extracted with CH₂Cl₂ (3×100 mL). Combined organic layer was dried over Na₂SO₄ and concentrated under reduced pressure to obtain compound 2 (9.5 g, 90%) as reddish brown thick syrup, which was taken next step without any further purification.

¹H-NMR (400 MHz, DMSO-d₆): δ 7.12 (d, J=3.6 Hz, 1H), 3.19-3.15 (m, 2H), 2.26-2.20 (m, 2H), 1.62-1.59 (m, 2H), 1.51-1.43 (m, 6H).

LCMS (ESI): m/z 128.1 [M+H]⁺

### Synthesis of 3,3-dichloroazocan-2-one (3)

To a solution of compound 2 (9.5 g, 74.6 mmol) in CH₂Cl₂ (19 mL) were added toluene (76 mL) and PCl₅ (31.1 g, 149.3 mmol) at room temperature under nitrogen atmosphere. The reaction mixture was heated to reflux and stirred for 2 h. After consumption of the starting material (by TLC), the reaction mixture was brought to room temperature and volatiles were evaporated under reduced pressure. Crude material was diluted with ice water (50 mL) and acetone (30 mL). Aqueous NaHCO₃ solution was added and pH was adjusted to 8 and then reaction mixture was extracted with CH₂Cl₂ (2×100 mL). Combined organic layer was dried over Na₂SO₄ and concentrated under reduced pressure. Obtained crude material was purified by silica gel column chromatography eluting 20% EtOAc/hexane to afford compound 3 (6.7 g, 46%) as white solid.

¹H-NMR: (500 MHz, DMSO-d₆): δ 7.92 (s, 1H), 3.41 (br s, 2H), 2.78 (s, 2H), 1.70-1.60 (m, 4H), 1.42-1.23 (m, 2H).

LCMS (ESI): m/z 196.1 [M+H]⁺.

### Synthesis of 3-chloroazocan-2-one (4)

To a stirring solution of compound 3 (2.6 g, 13.2 mmol) in methanol (39 mL) were added acetic acid (7.8 mL), sodium acetate (3 g, 36.5 mmol) and 10% Pd/C (650 mg) at room temperature under nitrogen atmosphere. The reaction mixture was stirred at room temperature for 2 h under H₂ atmosphere. After consumption of the starting material (by TLC), the reaction mixture was filtered through a pad of celite and volatiles were evaporated under reduced pressure. Aqueous NaHCO₃ solution was added and pH was adjusted to 8 and then reaction mixture was extracted with CH₂Cl₂ (2×50 mL). Combined organic layer was dried over Na₂SO₄ and concentrated under reduced pressure to obtain compound 4 (2.1 g, crude) as white solid, which was taken next step without any further purification.

¹H-NMR: (500 MHz, DMSO-d₆): δ 7.68 (s, 1H), 5.15-5.12 (m, 1H), 3.51-3.44 (m, 1H), 3.08-3.04 (m, 1H), 2.07-2.01 (m, 1H), 1.88-1.81 (m, 1H), 1.68-1.62 (m, 4H), 1.48-1.40 (m, 2H).

LCMS (ESI): m/z 162.1 [M+H]⁺.

### Synthesis of 1-(tert-butoxycarbonyl)azepane-2-carboxylic acid (5)

To a stirring solution of compound 4 (1.6 g, 9.9 mmol) in 1,4-dioxane (16 mL) was added NaOH (3.56 g, 89.1 mmol) and then heated to reflux for 16 h. The reaction mixture was cooled to 0° C., added water (8 mL) and Boc₂O (4.3 mL, 19.8 mmol) and allowed to stir for 5 h. After consumption of the starting material (by TLC), the reaction was diluted with water (10 mL) and extracted with CH₂Cl₂ (1×10 mL). Aqueous layer pH was adjusted to 2 using 2N HCl and then reaction mixture was extracted with CH₂Cl₂ (2×50 mL). The combined organic layer was dried over Na₂SO₄ and concentrated under reduced pressure to afford crude compound 5 (1.49 g, crude) as colorless thick syrup, which was taken next step without any further purification.

¹H-NMR: (500 MHz, DMSO-d₆): δ 12.56 (br s, 1H), 4.35-4.32 (m, 1H), 3.74-3.64 (m, 2H), 2.98-2.87 (m, 2H), 2.24-2.12 (m, 2H), 1.46-1.34 (m, 4H), 1.34 (s, 9H).

LCMS (ESI): m/z 241.8 [M−H]⁺.

### Synthesis of 1-(tert-butyl) 2-methyl azepane-1,2-dicarboxylate (6)

To a stirring solution of compound 5 (1.4 g, 5.7 mmol) in acetonitrile (14 mL) were added K₂CO₃ (2.38 g, 17.2 mmol) and Mel (0.72 mL, 11.5 mmol) at 0° C. under nitrogen atmosphere. The reaction mixture was brought to room temperature and allowed to stir for 16 h. After consumption of the starting material (by TLC), the reaction was diluted with water (20 mL) and extracted with EtOAc (2×30 mL). Combined organic layer was dried over Na₂SO₄ and concentrated under reduced pressure. Obtained crude material was purified by silica gel column chromatography eluting 10% EtOAc/hexane to afford compound 6 (720 mg, 49%) as colorless thick syrup.

¹H-NMR: (500 MHz, DMSO-d₆): δ 4.47-4.44 (m, 1H), 3.62 (s, 3H), 3.06-2.91 (m, 2H), 2.21-2.08 (m, 2H), 1.76-1.60 (m, 6H), 1.33 (s, 9H).

LCMS (ESI): m/z 158.2 [M-Boc+H]⁺.

### Synthesis of tert-butyl 1-oxo-2,5-diazaspiro[3.6]decane-5-carboxylate (EI-1 & EI-2)

To a stirring solution of compound 6 (760 mg, 2.9 mmol) in THF (7.6 mL) was added paraformaldehyde (106 mg, 3.5 mmol) at RT under nitrogen atmosphere. The reaction mixture was cooled to −78° C. and added LiHMDS (8.8 mL, 8.8 mmol) and allowed to stir at room temperature for 4 h. After consumption of the starting material (by TLC), the reaction was quenched with water (10 mL) and extracted with EtOAc (2×20 mL). The combined organic layer was washed with water (2×15 mL) followed by brine solution (2×10 mL). The organic layer was dried over Na₂SO₄ and concentrated to obtain crude material which was purified by column chromatography by eluting 40% EtOAc/hexane to afford a racemic mixture of EI-1 & EI-2 (450 mg, 60%) as white solid. The racemic was separated by chiral HPLC purification and obtained 150 mg of EI-1 and 160 mg of EI-2.

### EI-1

¹H-NMR: (400 MHz, DMSO-d₆): δ 7.82 (s, 1H), 3.67-3.61 (m, 1H), 3.34-3.26 (m, 2H), 3.06 (d, J=5.6 Hz, 1H), 2.20-2.13 (m, 1H), 1.98-1.95 (m, 1H), 1.78-1.54 (m, 4H), 1.40-1.38 (m, 1H), 1.39 (s, 9H), 1.29-1.21 (m, 1H).

LCMS (ESI): m/z 153.1 [M-Boc+H]⁺

HPLC: 99.72%

### EI-2

¹H-NMR: (400 MHz, DMSO-d₆): δ 7.82 (s, 1H), 3.67-3.61 (m, 1H), 3.34-3.24 (m, 2H), 3.06 (d, J=5.6 Hz, 1H), 2.20-2.13 (m, 1H), 1.98-1.95 (m, 1H), 1.78-1.54 (m, 4H), 1.40-1.38 (m, 1H), 1.39 (s, 9H), 1.28-1.21 (m, 1H).

LCMS (ESI): m/z 153.1 [M-Boc+H]⁺

HPLC: 99.77%

Following the above procedures, the following compounds and stereoisomers thereof were or are prepared. It will be appreciated by a person of skill in the art that for structures shown additional diastereomers and/or enantiomers may be envisioned and are included herein.

TABLE 2

| Compound | Structure |
|---|---|
| GA | 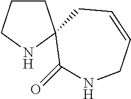 [SMILES: O=C1NCC=CC[C@]12CCCN2] |
| GB-1, GB-2 | 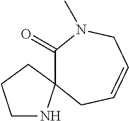 |
| GC-1, GC-2 | 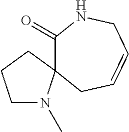 |
| GD-1, GD-2 | 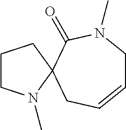 |
| GE-1, GE-2 | 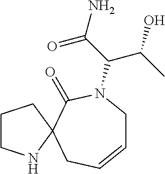 |
| GF-1, GF-2 | 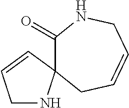 [SMILES: O=C1NCC=CCC12C=CCN2] |
| GG-1, GG-2 | 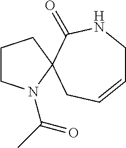 |
| GH-1, GH-2 | 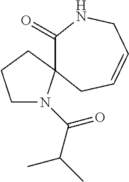 [SMILES: CC(C)C(=O)N1CCCC12CC=CCNC2=O] |
| GI-1, GI-2 | 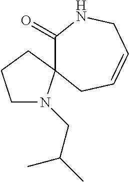 [SMILES: CC(C)CN1CCCC12CC=CCNC2=O] |
| GJ-1, GJ-2 | 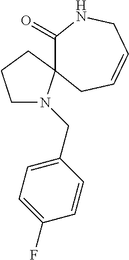 [SMILES: O=C1NCC=CCC12CCCN2Cc1ccc(F)cc1] |
| GK-1, GK-2 | 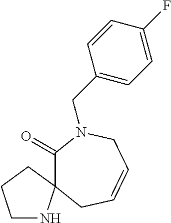 [SMILES: O=C1N(Cc2ccc(F)cc2)CC=CCC12CCCN2] |
| GL-1, GL-2 | 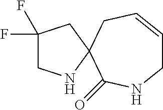 [SMILES: O=C1NCC=CCC12CC(F)(F)CN2] |
| GM-1, GM-2 | 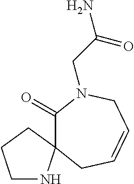 [SMILES: NC(=O)CN1CC=CCC2(CCCN2)C1=O] |
| GN-1, GN-2 | 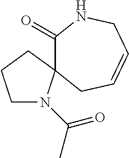 |
| GO-1, GO-2 | 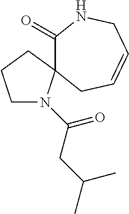 [SMILES: CC(C)CC(=O)N1CCCC12CC=CCNC2=O] |
| GP-1, GP-2 | 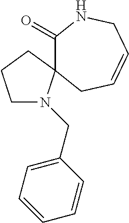 [SMILES: O=C1NCC=CCC12CCCN2Cc1ccccc1] |
| GQ-1, GQ-2 | 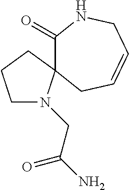 [SMILES: NC(=O)CN1CCCC12CC=CCNC2=O] |
| GR-1, GR-2 | 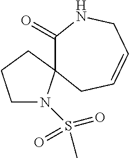 [SMILES: CS(=O)(=O)N1CCCC12CC=CCNC2=O] |
| GS-1, GS-2 | 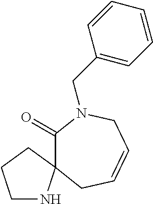 [SMILES: O=C1N(Cc2ccccc2)CC=CCC12CCCN2] |
| GT-1, GT-2 | 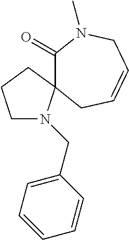 [SMILES: CN1CC=CCC2(CCCN2Cc2ccccc2)C1=O] |
| GU-1, GU-2 | 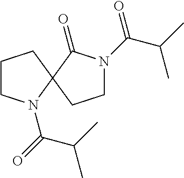 [SMILES: CC(C)C(=O)N1CCC2(CCCN2C(=O)C(C)C)C1=O] |
| GV-1, GV-2 | 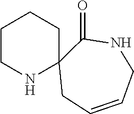 [SMILES: O=C1NCC=CCC12CCCCN2] |
| GW | 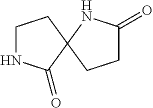 [SMILES: O=C1CCC2(CCNC2=O)N1] |
| GX | 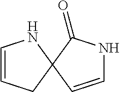 |
| GY | 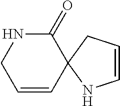 [SMILES: O=C1NCC=CC12CC=CN2] |
| GZ | 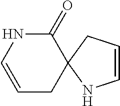 [SMILES: O=C1NC=CCC12CC=CN2] |
| HA | 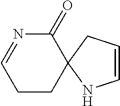 [SMILES: O=C1N=CCCC12CC=CN2] |

### B. NMDAR Agonist Assays

Assays were conducted as described by Moskal et al., “GLYX-13: a monoclonal antibody-derived peptide that acts as an N-methyl-D-aspartate receptor modulator,” Neuropharmacology, 49, 1077-87, 2005. These studies were designed to determine if the test compounds act to facilitate NMDAR activation in NMDAR2A, NMDAR2B, NMDAR2C or NMDAR2D expressing HEK cell membranes as measured by increases in [³H]MK-801 binding.

In the assay, 300 μg of NMDAR expressing HEK cell membrane extract protein was preincubated for 15 minutes at 250° C. in the presence of saturating concentrations of glutamate (50 μM) and varying concentrations of test compound (1×10⁻¹⁵M-1×10⁻⁷M), or 1 mM glycine. Following the addition of 0.3 μCi of [3H]MK-801 (22.5 Ci/mmol), reactions were again incubated for 15 minutes at 25° C. (nonequilibrium conditions). Bound and free [3H]MK-801 were separated via rapid filtration using a Brandel apparatus.

In analyzing the data, the DPM (disintegrations per minute) of [3H]MK-801 remaining on the filter were measured for each concentration of test compound or for 1 mM glycine. The DPM values for each concentration of a ligand (N=2) were averaged. The baseline value was determined from the best fit curve of the DPM values modeled using the GraphPad program and the log(agonist) vs. response (three parameters) algorithm was then subtracted from all points in the dataset. The % maximal [³H]MK-801 binding was then calculated relative to that of 1 mM glycine: all baseline subtracted DPM values were divided by the average value for 1 mM glycine. The EC₅₀ and % maximal activity were then obtained from the best fit curve of the % maximal [3H]MK-801 binding data modelled using the GraphPad program and the log(agonist) vs. response (three parameters) algorithm.

The tables below summarize the results for the wild type NMDAR agonists NMDAR2A, NMDAR2B, NMDAR2C, and NMDAR2D, and whether the compound is not an agonist (−), is an agonist (+), or is a strong agonist (++), where column A is based on the % maximal [3H]MK-801 binding relative to 1 mM glycine (−=0; <100%=+; and >100%=++); and column B is based on log EC₅₀ values (0=−; ≥1×10⁻⁹ M (e.g., −8)=+; and <1×10⁻⁹ M (e.g., −10)=++). An “ND” indicates that the assay was not done.

| | |
|---|---|
| NMDAR2A | NMDAR2B |

| Compound | A | B | A | B |
|---|---|---|---|---|
| EI-1 | + | ++ | ND | ND |
| EI-2 | + | ++ | ND | ND |
| EU-1 | − | − | + | ++ |
| EU-2 | − | − | + | ++ |
| AU-1 | + | + | + | ++ |
| EV-1 | + | ++ | + | ++ |
| EV-2 | + | ++ | + | ++ |
| AU-2 | − | − | + | ++ |

| | |
|---|---|
| NMDAR2A | NMDAR2B |

| Compound | A | B | A | B |
|---|---|---|---|---|
| GA | + | + | + | + |
| GP-1 | + | ++ | ++ | ++ |
| GP-2 | ++ | ++ | + | ++ |
| GJ-1 | + | ++ | + | ++ |
| GJ-2 | + | ++ | + | ++ |
| GQ-1 | + | ++ | ++ | + |
| GQ-2 | + | ++ | + | ++ |
| GS-1 | + | ++ | + | ++ |
| GS-2 | + | + | + | ++ |
| GK-1 | + | ++ | ++ | ++ |
| GK-2 | − | − | + | ++ |
| GH-1 | + | ++ | + | ++ |
| GH-2 | + | ++ | ++ | ++ |
| GT-1 | + | ++ | + | ++ |
| GT-2 | + | ++ | + | + |
| GI-1 | + | ++ | + | ++ |
| GI-2 | + | ++ | + | ++ |
| GC-1 | + | ++ | ++ | ++ |
| GC-2 | + | + | + | ++ |
| GL-1 | + | ++ | + | ++ |
| GL-2 | + | ++ | + | ++ |
| GG-1 | + | ++ | + | ++ |
| GG-2 | + | ++ | + | ++ |
| GD-1 | − | − | − | − |
| GD-2 | + | ++ | − | − |
| AX | + | ++ | + | + |
| AY | + | ++ | + | ++ |
| AZ | + | ++ | + | ++ |
| BA | + | ++ | + | ++ |
| CD | + | ++ | + | ++ |

| | |
|---|---|
| NMDAR2A | NMDAR2B |

| Compound | A | B | A | B |
|---|---|---|---|---|
| CE | + | ++ | + | ++ |
| CF | + | ++ | + | ++ |
| CG | + | ++ | + | ++ |
| GU-1 | 0 | 0 | + | ++ |
| GU-2 | + | ++ | + | ++ |

| | |
|---|---|
| NMDAR2C | NMDAR2D |

| Compound | A | B | A | B |
|---|---|---|---|---|
| EI-1 | ND | ND | + | ++ |
| EU-2 | + | ++ | ++ | ++ |
| EV-1 | + | ++ | + | ++ |
| EV-2 | + | ++ | + | ++ |
| AU-2 | + | ++ | + | ++ |
| GA | 0 | 0 | + | ++ |
| AU-1 | ++ | ++ | 0 | 0 |

### C. Pharmacokinetics Assays

Sprague Dawley rats were dosed intravenously using a normal saline formulation containing 2 mg/kg of the compounds identified in the below table. The table below summarizes the results of the IV pharmacokinetics.

| | | | | Cl | |
|---|---|---|---|---|---|
| | Cₘₐₓ | AUCₗₐₛₜ | T_{1/2} | (mL/min/ | Vₛₛ |
| Compound | (ng/mL) | (hr*ng/mL) | (hr) | kg) | (L/kg) |
| EU-2 | 2143 | 4001.6 | 1.61 | 8.79 | 0.94 |
| AU-1 | 1625 | 1843 | 1.41 | 17.84 | 1.63 |
| EV-1 | 2730 | 1271.4 | 0.42 | 26.2 | 0.72 |
| EV-2 | 2608.4 | 1138.1 | 0.41 | 28.4 | 0.88 |
| GA | 1466.1 | 1245.4 | 3.6 | 27 | 3.92 |
| GH-2 | 1496.49 | 1989.18 | 0.43 | 16.73 | 0.75 |
| GT-1 | 420.81 | 210.32 | 0.66 | 156.89 | 6.5 |
| GC-1 | 1207.17 | 1020.58 | 0.68 | 32.21 | 1.78 |
| GQ-2 | 2310.43 | 1294.69 | 1.77 | 25.71 | 1.11 |
| GK-1 | 854.13 | 335.22 | 0.54 | 98.36 | 3.01 |
| GI-2 | 975.87 | 325.87 | 0.32 | 101.37 | 2.25 |
| GL-2 | 1634.35 | 11507.99 | 4.07 | 2.84 | 1.15 |
| GG-2 | 3422.47 | 2547.7 | 0.54 | 13.13 | 0.58 |
| GU-1 | 5852.73 | 1138.79 | 0.18 | 29.29 | 0.36 |
| AX | 1420.18 | 1358.37 | 9.15 | 24.39 | 3.31 |
| AY | 1552.63 | 1658.09 | 9.65 | 20.15 | 3.88 |
| AZ | 17901.75 | 15355.97 | 4.25 | 2.17 | 0.17 |
| CF | 1989.28 | 1106.45 | 1.8 | 29.92 | 2.85 |

In another experiment, Sprague Dawley rats were dosed per os (oral gavage) using a normal saline formulation containing 10 mg/kg of the compounds identified in the table below. Plasma, brain, and CSF samples were analyzed at various time points over a 24 hour period. The table below summarizes the results of the oral pharmacokinetics, where the first three values (Tₘₐₓ, Cₘₐₓ and AUCₗₐₛₜ) are plasma values.

| | | | AUC₍₀₋ₗₐₛₜ₎ | CSF | Brain | |
|---|---|---|---|---|---|---|
| | Tₘₐₓ | Cₘₐₓ | (hr*ng/ | Cₘₐₓ | Cₘₐₓ | |
| Compound | (hr) | (ng/mL) | mL) | (ng/mL) | (ng/mL) | % F |
| EU-2 | 1 | 1862.3 | 4433.2 | 436.7 | 593.1 | 100 |
| AU-1 | 1 | 2438 | 6955 | 199.26 | 514.7 | 75 |
| EV-1 | 0.25 | 3689.4 | 2847.3 | 1414.8 | 1457.4 | 45 |
| EV-2 | 0.25 | 4346 | 3567.9 | 2324.1 | 1213 | 63 |
| GA | 0.5 | 1964.8 | 3857.5 | 551.3 | 3963.2 | 62 |
| GH-2 | 0.42 | 5300.84 | 11329.22 | 2296.73 | 1424.03 | 100 |
| GT-1 | 0.58 | 23.31 | 9.16 | 0 | 0 | 1 |
| GC-1 | 0.42 | 2399.16 | 4426.53 | 744.03 | 2578.7 | 87 |
| GQ-2 | 0.25 | 5385.58 | 5475.44 | 1400.31 | 492.96 | 85 |
| GK-1 | 0.5 | 642.38 | 718.25 | 322.45 | 3368.95 | 43 |
| GI-2 | 0.42 | 132.5 | 150.25 | 205.53 | 97.39 | 9 |
| GL-2 | 0.5 | 5820.33 | 30506.19 | 4433.95 | 3556.03 | 53 |
| GG-2 | 0.25 | 9200.58 | 19585.84 | 2271.94 | 1689.69 | 100 |
| GU-1 | 0.25 | 4617.11 | 2099.64 | 0 | 0 | 37 |
| AX | 1 | 2564 | 6676.86 | 342.07 | 1547.36 | 98 |
| AY | 1 | 3038.07 | 6528.55 | 701.8 | 799.5 | 79 |
| AZ | 1 | 29116.3 | 74253.1 | 5343.89 | 8074.7 | 97 |
| CF | 2 | 555.99 | 1833.09 | 51.64 | 48.22 | 33 |

### D: Porsolt Assay

A non-clinical in vivo pharmacology study (Porsolt assay) was performed to measure antidepressant-like effects. The study allowed for the evaluation of the effects of each compound on the Porsolt forced swim test as assessed by the rats' response (reduced floating time) during a 5-minute swimming test.

Male 2-3 month old Sprague Dawley rats were used (Harlan, Indianapolis, Ind.). Rats were housed in Lucite cages with aspen wood chip bedding, maintained on a 12:12 light:dark cycle (lights on at 5 AM), and given ad libitum access to Purina lab chow (USA) and tap water throughout the study.

The Porsolt forced swim test adapted for use in rats was performed as described by Burgdorf et al., (The long-lasting antidepressant effects of rapastinel (GLYX-13) are associated with a metaplasticity process in the medial prefrontal cortex and hippocampus. Neuroscience 308:202-211, 2015). Animals were placed in a 46 cm tall×20 cm in diameter clear glass tube filled to 30 cm with tap water (23±1° C.) for 15 min on the first day (habituation) and 5 min on the subsequent test day. Animals were tested 1 h or 24 h post-dosing with the test compounds or vehicle control (0.5% sodium carboxymethyl cellulose in 0.9% sterile saline). A subset of compounds tested at 1 h post-dosing was tested again 1 wk post-dosing. Animals received a 15 min habituation session 1 day before the first 5 min test. Water was changed after every other animal. Animals were videotaped, and floating time as defined as the minimal amount of effort required to keep the animals head above water was scored offline by a blinded experimenter with high inter-rater reliability (Pearson's r >0.9). An “ND” indicates that the assay was not done.

| | 1 h post-dose | | | 24 h post-dose | | | 1 wk post-dose | | |
|---|---|---|---|---|---|---|---|---|---|
| | | | % | | | % | | | % |
| | | | reduction | | | reduction | | | reduction |
| | Dose | Significance | in float | Dose | Significance | in float | Dose | Significance | in float |
| Compound | (mg/kg) | vs. vehicle | time | (mg/kg) | vs. vehicle | time | (mg/kg) | vs. vehicle | time |
| EU-2 | 0.1 | Yes | 55 | ND | ND | ND | 0.1 | Yes | 46 |
| EV-1 | 0.1 | Yes | 53 | 0.1 | Yes | 45 | ND | ND | ND |
| EV-2 | 0.1 | Yes | 84 | 0.1 | Yes | 76 | ND | ND | ND |
| GA | 0.00001 | No | 29 | ND | ND | ND | 0.00001 | Yes | 55 |
| GA | 0.001 | Yes | 58 | ND | ND | ND | 0.001 | Yes | 71 |
| GA | 0.1 | Yes | 67 | 0.1 | Yes | 64 | 0.1 | Yes | 72 |
| GA | 10.0 | No | 50 | ND | ND | ND | 10.0 | Yes | 44 |
| AU-1 | 0.1 | Yes | 86 | ND | ND | ND | ND | ND | ND |
| GK-1 | 0.1 | No | 24 | ND | ND | ND | ND | ND | ND |
| GL-2 | 0.1 | No | 6 | ND | ND | ND | ND | ND | ND |
| GG-2 | 0.1 | No | 4 | ND | ND | ND | ND | ND | ND |

### E. Microsomal Stability

Microsomal stability of disclosed compounds was investigated. The following table indicates the percent of compound remaining after 60 minutes.

| Compound | Microsomal (Human) | Microsomal (Rat) |
|---|---|---|
| EU-2 | 100% | 94% |
| AU-1 | 100% | 91% |
| EV-1 | 95% | 100% |
| EV-2 | 79% | 91% |
| GA | 94% | 90% |
| GP-1 | 80% | 2% |
| GP-2 | 89% | 7% |
| GJ-1 | 83% | 11% |
| GJ-2 | 95% | 3% |
| GQ-1 | 88% | 87% |
| GQ-2 | 94% | 102% |
| GS-1 | 99% | 0% |
| GS-2 | 90% | 0% |
| GK-2 | 94% | 46% |
| GH-1 | 104% | 100% |
| GH-2 | 87% | 104% |
| GT-1 | 72% | 3% |
| GI-1 | 96% | 69% |
| GI-2 | 114% | 102% |
| GC-1 | 115% | 101% |
| GC-2 | 82% | 102% |
| GC-1 | 67% | 29% |
| GC-2 | 100% | 29% |
| GL-2 | 99% | 98% |
| GG-2 | 88% | 106% |
| GU-1 | 96% | 94% |
| AX | 93% | 120% |
| AY | 97% | 114% |
| AZ | 92% | 91% |
| CF | 91% | 88% |

### F. Plasma Stability

Plasma stability of disclosed compounds was investigated. The following table indicates the percent of compound remaining after 60 minutes.

| Compound | Plasma (Human) | Plasma (Rat) |
|---|---|---|
| EU-2 | 74% | 95% |
| AU-1 | 100% | 100% |
| EV-1 | 98% | 94% |
| EV-2 | 100% | 79% |
| GA | 95% | 100% |
| GP-1 | 98% | 96% |
| GP-2 | 96% | 85% |
| GJ-1 | 105% | 100% |
| GJ-2 | 95% | 99% |
| GQ-1 | 98% | 98% |
| GQ-2 | 96% | 97% |
| GS-1 | 97% | 95% |
| GS-2 | 98% | 99% |
| GK-2 | 92% | 97% |
| GH-1 | 103% | 102% |
| GH-1 | 96% | 102% |
| GT-1 | 101% | 96% |
| GI-1 | 96% | 102% |
| GI-2 | 99% | 104% |
| GC-1 | 97% | 89% |
| GC-2 | 98% | 103% |
| GL-2 | 91% | 97% |
| GG-2 | 99% | 101% |
| GU-1 | 82% | 89% |
| AX | 105% | 99% |
| AY | 104% | 107% |
| AZ | 101% | 101% |
| CF | 107% | 91% |

### EQUIVALENTS

Those skilled in the art will recognize, or be able to ascertain using no more than routine experimentation, many equivalents to the specific embodiments of the invention described herein. Such equivalents are intended to be encompassed by the following claims.

### INCORPORATION BY REFERENCE

The entire contents of all patents, published patent applications, websites, and other references cited herein are hereby expressly incorporated herein in their entireties by reference.

1. A compound represented by a formula selected from the group consisting of: 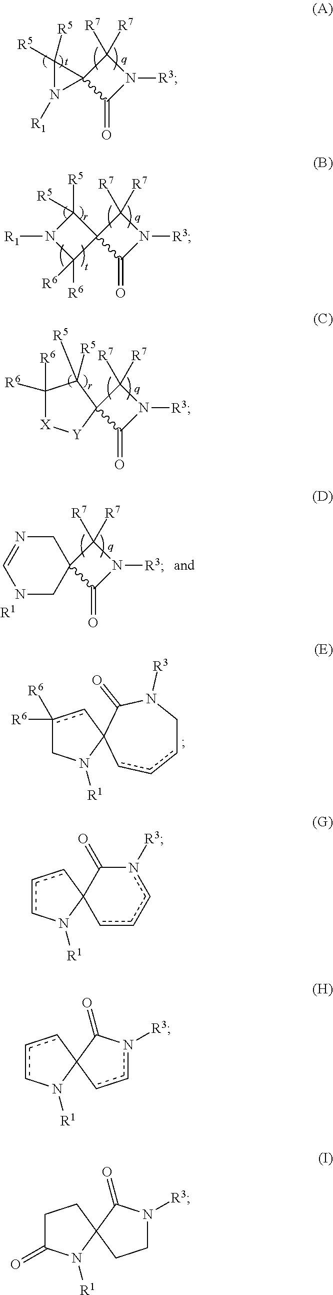
or a pharmaceutically acceptable salt and/or a stereoisomer thereof, wherein
R¹ is independently selected from the group consisting of H, —C₁-C₆alkyl, —C(O)—C₁-C₆alkyl, —C(O)—O—C₁-C₆alkyl, and —S(O)_{w}—C₁-C₆alkyl, wherein C₁-C₆alkyl is optionally substituted with one, two, or three substituents each independently selected from R^{S};
w is 0, 1 or 2;
R⁵ is independently selected for each occurrence from the group consisting of H, —C₁-C₆alkyl, and halogen, wherein C₁-C₆alkyl is optionally substituted with one, two, or three substituents each independently selected from R^{S};
R⁶ is independently selected for each occurrence from the group consisting of H, —C₁-C₆alkyl, and halogen, wherein C₁-C₆alkyl is optionally substituted with one, two, or three substituents each independently selected from R^{S}; or
R⁵ and R⁶, or two R⁵ moieties, when present on adjacent carbons, form a 3-membered carbocyclic ring taken together with the adjacent carbons to which they are attached, optionally substituted by one or two substituents independently selected from the group consisting of halogen, hydroxyl, —C₁-C₃alkyl, —C₁-C₃alkoxy, —C(O)NR^{a}R^{b}, and —NR^{a}R^{b};
R⁷ is independently selected for each occurrence from the group consisting of H, —C₁-C₆alkyl, phenyl, and halogen, wherein C₁-C₆alkyl is optionally substituted with one, two, or three substituents each independently selected from R^{S}, and phenyl is optionally substituted with one, two, or three substituents each independently selected from R^{T};
R³ is selected from the group consisting of H, —C₁-C₆alkyl, phenyl, —C(O)—R³¹, and —C(O)—O—R³², wherein C₁-C₆alkyl is optionally substituted with one, two, or three substituents each independently selected from R^{S}, and phenyl is optionally substituted with one, two, or three substituents each independently selected from R^{T};
R³¹ is selected from the group consisting of H, —C₁-C₆alkyl, —C₃-C₆cycloalkyl, and phenyl, wherein C₁-C₆alkyl is optionally substituted with one, two, or three substituents each independently selected from R^{S}, and each of C₃-C₆cycloalkyl and phenyl is optionally substituted with one, two, or three substituents each independently selected from R^{T};
R³² is selected from the group consisting of H, —C₁-C₆alkyl, —C₃-C₆cycloalkyl, and phenyl, wherein C₁-C₆alkyl is optionally substituted with one, two, or three substituents each independently selected from R^{S}, and phenyl is optionally substituted with one, two, or three substituents each independently selected from R^{T}; and
R^{a} and R^{b} are independently, for each occurrence, selected from the group consisting of H, —C(O)—O—CH₂-phenyl, and —C₁-C₃alkyl; or R^{a} and R^{b} taken together with the nitrogen to which they are attached form a 4-6 membered heterocyclic ring, wherein phenyl is optionally substituted with one, two, or three substituents each independently selected from R^{T};
R^{S} is independently, for each occurrence, selected from the group consisting of —C(O)NR^{a}R^{b}, —NR^{a}R^{b}, hydroxyl, —SH, phenyl, —O—CH₂-phenyl, and halogen, wherein each phenyl is optionally substituted with one, two, or three substituents each independently selected from the group consisting of —C₁-C₃alkoxy and halogen;
R^{T} is independently, for each occurrence, selected from the group consisting of —C(O)NR^{a}R^{b}, —NR^{a}R^{b}, —C₁-C₃alkyl, —C₁-C₃alkoxy, hydroxyl, and halogen; and
wherein
for Formula A:
t is 1, and q is 1, 2, 3, 4, or 5; or;
t is 2, 4 or 5, and q is 2, 3, 4, or 5; or,
t is 3 and q is 3, 4, or 5;
for Formula B:
t is 1, r is 1, and q is 1, 2, 3, 4, or 5; or
t is 1, r is 2, and q is 1, 3, 4, or 5, or
t is 1, r is 3, q is 3, 4, or 5, or
t is 1, r is 4, q is 2, 3, 4, or 5; or
t is 2, r is 3 or 4, q is 2, 3, 4, or 5;
for Formula C:
r is 0, 1, or 2;
q is 1, 2, 3, 4, or 5; and
—X—Y— is selected from the group consisting of: 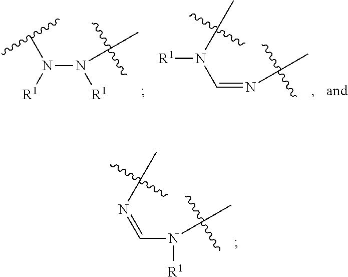
for Formula D:
q is 1, 2, 3, 4, or 5; and
for Formula E: [symbol]
is either a single or double bond;
when a double bond is present in the 5-membered ring, only one R⁶ is present;
the one double bond in the 7-membered ring is present between the α and β ring carbons or the β and γ ring carbons, with respect to the spiro junction;
for Formula G: [symbol]
is either a single or double bond;
there is one double bond in the 5-membered ring;
there is one double bond in the 6-membered ring;
if the double bond in the 6-membered ring is a C═N bond, then R³ is absent;
for Formula H: [symbol]
is either a single or double bond;
there is one double bond in the ring without a carbonyl group;
there is one double bond in the ring with a carbonyl group; and
if the double bond in the ring with a carbonyl group is a C═N bond, then R³ is absent.

2. The compound of claim 1, wherein R¹ is H.

3-14. (canceled)

15. The compound of claim 1, wherein R³ is H.

16-19. (canceled)

20. A compound represented by 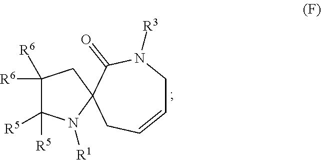
or a pharmaceutically acceptable salt and/or a stereoisomer thereof, wherein
R¹ is independently selected from the group consisting of H, —C₁-C₄alkyl, —C(O)—C₁-C₄alkyl, —S(O)—C₁-C₄alkyl, and —C(O)—O—C₁-C₄alkyl, wherein C₁-C₄alkyl is optionally substituted with one, two, or three substituents each independently selected from R^{S};
w is 0, 1 or 2;
R⁵ is independently selected for each occurrence from the group consisting of H, —C₁-C₄alkyl, and halogen, wherein C₁-C₄alkyl is optionally substituted with one, two, or three substituents each independently selected from R^{S};
R⁶ is independently selected for each occurrence from the group consisting of H, —C₁-C₄alkyl, and halogen, wherein C₁-C₄alkyl is optionally substituted with one, two, or three substituents each independently selected from R^{S};
R³ is selected from the group consisting of H, —C₁-C₄alkyl, —C₁-C₄alkyl-phenyl, —C(O)—R³¹, and —C(O)—O—R³², wherein C₁-C₄alkyl is optionally substituted with one, two, or three substituents each independently selected from R^{S}, and phenyl is optionally substituted with one, two, or three substituents each independently selected from R^{T};
R³¹ is selected from the group consisting of H, —C₁-C₄alkyl, —C₃-C₆cycloalkyl, and phenyl, wherein C₁-C₄alkyl is optionally substituted with one, two, or three substituents each independently selected from R^{S}, and phenyl is optionally substituted with one, two, or three substituents each independently selected from R^{T};
R³² is selected from the group consisting of H, —C₁-C₄alkyl, —C₃-C₆cycloalkyl, and phenyl, wherein C₁-C₄alkyl is optionally substituted with one, two, or three substituents each independently selected from R^{S}, and phenyl is optionally substituted with one, two, or three substituents each independently selected from R^{T}; and
R^{a} and R^{b} are each independently for each occurrence selected from the group consisting of H, phenyl, and —C₁-C₄alkyl; or R^{a} and R^{b} taken together with the nitrogen to which they are attached form a 4-6 membered heterocyclic ring, wherein C₁-C₄alkyl is optionally substituted with one, two, or three substituents each independently selected from —C₁-C₃alkoxy, hydroxyl, and halogen;
R^{S} is independently, for each occurrence, selected from the group consisting of —C(O)NR^{a}R^{b}, —NR^{a}R^{b}, hydroxyl, —C(O)—O—R^{a}, phenyl, and halogen, wherein each phenyl is optionally substituted with one, two, or three substituents each independently selected from the group consisting of —C₁-C₃alkoxy and halogen; and
R^{T} is independently, for each occurrence selected from the group consisting of —C(O)NR^{a}R^{b}, —NR^{a}R^{b}, —C₁-C₃alkoxy, hydroxyl, and halogen.

21. The compound of claim 20, wherein R¹ is H.

22. The compound of claim 20, wherein R¹ is methyl.

23. The compound of claim 20, wherein R¹ is —CH₂-phenyl, optionally substituted by halogen.

24. The compound of claim 20, wherein R¹ is —C(O)—C₁-C₄alkyl.

25. The compound of claim 24, wherein R¹ is —C(O)CH(CH₃)₂.

26. The compound of claim 20, wherein R¹ is —CH₂C(O)NH₂.

27-28. (canceled)

29. The compound of claim 20, wherein each R⁵ and R⁶ is H.

30. The compound of claim 20, wherein R³ is H.

31. The compound of claim 20, wherein R³ is methyl.

32. The compound of claim 20, wherein R³ is 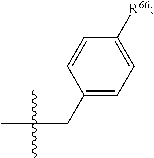
wherein R⁶⁶ is selected from the group consisting of H, halogen and —C₁-C₃alkoxy.

33. The compound of claim 32, wherein R⁶⁶ is F.

34. A compound selected from the group consisting of 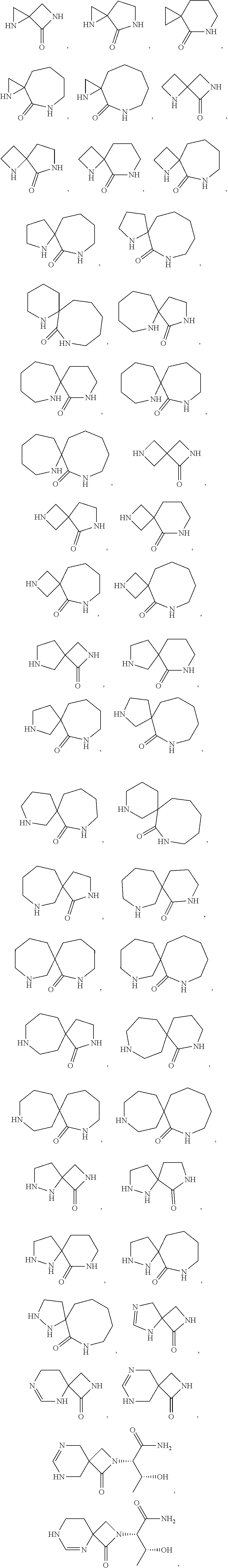 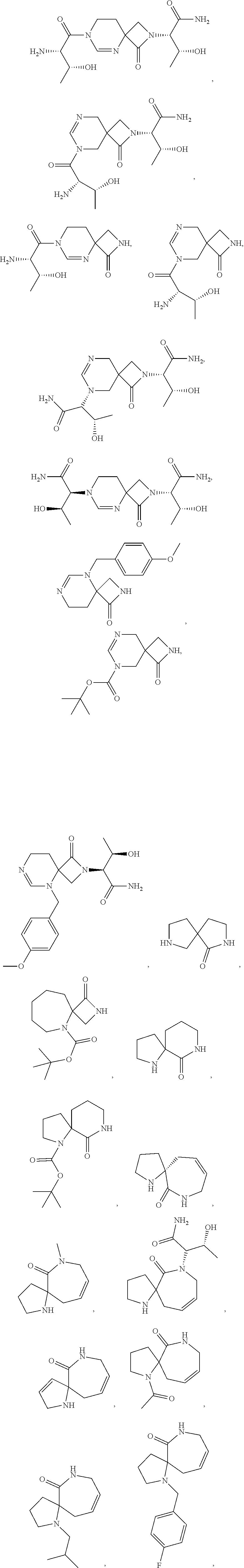 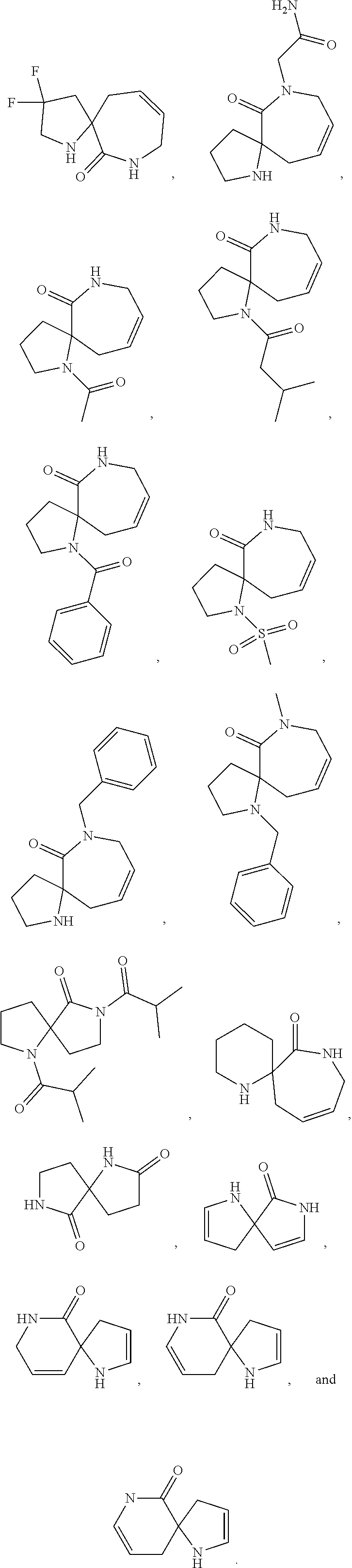
or a pharmaceutically acceptable salt and/or a stereoisomer thereof.

35. A compound of claim 20 selected from the group consisting of: 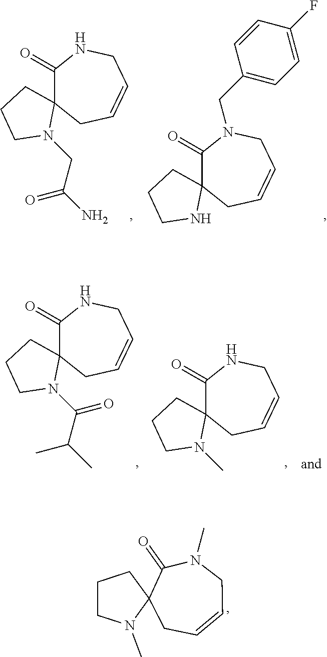
or a pharmaceutically acceptable salt and/or a stereoisomer thereof.

36. A pharmaceutical composition comprising the compound of claim 1, and a pharmaceutically acceptable excipient.

37. (canceled)

38. A method of treating of treating migraine, neuropathic pain, traumatic brain injury, a neurodevelopmental disorder related to synaptic dysfunction, a cognitive impairment disorder, depression, Alzheimer's disease, attention deficit disorder, schizophrenia, or anxiety, in a patient in need thereof, comprising administering to the patient an effective amount of the compound of claim 1.

39-43. (canceled)

44. The compound of claim 1 selected from 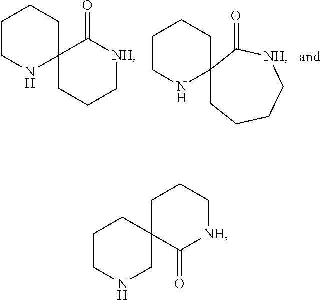
or a pharmaceutically acceptable salt and/or a stereoisomer thereof.
